(19) United States
(12) Patent Application Publication
(10) Pub. No.: US 2024/0192592 A1
(43) Pub. Date: Jun. 13, 2024

(54) ACTINIC RAY-SENSITIVE OR RADIATION-SENSITIVE RESIN COMPOSITION, ACTINIC RAY-SENSITIVE OR RADIATION-SENSITIVE FILM, PATTERN FORMING METHOD, METHOD FOR MANUFACTURING ELECTRONIC DEVICE, AND COMPOUND

(71) Applicant: FUJIFILM Corporation, Tokyo (JP)

(72) Inventors: Takeshi KAWABATA, Haibara-gun (JP); Minoru UEMURA, Haibara-gun (JP); Tomotaka TSUCHIMURA, Haibara-gun (JP)

(73) Assignee: FUJIFILM Corporation, Tokyo (JP)

(21) Appl. No.: 18/421,949

(22) Filed: Jan. 24, 2024

Related U.S. Application Data
(63) Continuation of application No. PCT/JP22/28539, filed on Jul. 22, 2022.

(30) Foreign Application Priority Data
Jul. 29, 2021 (JP) 2021-124857

(51) Int. Cl. G03F 7/004; C07C 25/18; C07C 43/225; C07C 309/07; C07C 309/11; C07C 309/12; C07C 309/17; C07C 311/09; C07C 381/12; C07D 327/06; G03F 7/038; G03F 7/039

(52) U.S. Cl. CPC G03F 7/0045; C07C 25/18; C07C 43/225; C07C 309/07; C07C 309/11; C07C 309/12; C07C 309/17; C07C 311/09; C07C 381/12; C07D 327/06; G03F 7/038; G03F 7/039

(57) ABSTRACT

An actinic ray-sensitive or radiation-sensitive resin composition contains a compound (I) that generates an acid upon irradiation with actinic rays or radiation and has an ionic structure and a zwitterionic structure. A pattern forming method contains forming an actinic ray-sensitive or radiation-sensitive film on a substrate using the actinic ray-sensitive or radiation-sensitive resin composition, exposing the actinic ray-sensitive or radiation-sensitive film to light; and developing the exposed actinic ray-sensitive or radiation-sensitive film with a developer.

### CROSS REFERENCE TO RELATED APPLICATION

This is a continuation of International Application No. PCT/JP2022/028539 filed on Jul. 22, 2022, and claims priority from Japanese Patent Application No. 2021-124857 filed on Jul. 29, 2021, the entire disclosures of which are incorporated herein by reference.

### BACKGROUND OF THE INVENTION

### 1. Field of the Invention

The present invention relates to an actinic ray-sensitive or radiation-sensitive resin composition, an actinic ray-sensitive or radiation-sensitive film, a pattern forming method, a method for manufacturing an electronic device, and a compound. More particularly, the invention relates to an actinic ray-sensitive or radiation-sensitive resin composition that is preferably used for ultramicrolithography processes applicable to processes for manufacturing VLSI (very large scale integration) circuits and high-capacity microchips, to processes for producing molds for nanoimprinting, and to processes for manufacturing high-density information recording mediums and also applicable to other photofabrication processes and also relates to an actinic ray-sensitive or radiation-sensitive film, a pattern forming method, a method for manufacturing an electronic device, and a compound.

### 2. Description of the Related Art

Conventional manufacturing processes for semiconductor devices such as ICs (Integrated Circuits) or LSI circuits involve lithographic microfabrication using a photoresist composition. In recent years, the degree of integration of integrated circuits has increased, and there is a growing demand for ultra-fine pattern formation in the submicron or quarter-micron range. Accordingly, the wavelength of exposure light tends to be shortened. Specifically, the g-line is replaced by the i-line and then by KrF excimer laser light. At present, exposure devices using an ArF excimer laser with a wavelength of 193 nm as the light source are being developed. In addition, techniques for further increasing resolving power are under development. Specifically, the so-called immersion method is under development, in which the space between a projection lens and a specimen is filled with a high-refractive index liquid (hereinafter referred to as “immersion liquid”).

At present, in addition to the use of excimer laser light, lithography using electron beams (EB), X-rays, extreme ultraviolet rays (EUV), etc. is being developed. This leads to the development of chemical amplification resist compositions that are effectively sensitive to various types of radiation and exhibit high sensitivity and resolution.

For example, JP2013-167825A and WO2013/121819A describe an actinic ray-sensitive or radiation-sensitive resin composition including a compound that is represented by specific general formula (Z1) and generates an acid upon irradiation with actinic rays or radiation.

JP2012-189977A describes a resist composition including: a compound represented by specific formula (I); a resin that is insoluble or poorly soluble in an alkali aqueous solution but becomes soluble in an alkali aqueous solution by the action of an acid; and an acid generator.

### SUMMARY OF THE INVENTION

In recent years, patterns are being reduced in size, and there is a need for further improvement in the performance of actinic ray-sensitive or radiation-sensitive resin compositions used to form such patterns.

With the conventional techniques described in JP2013-167825A, WO2013/121819A, and JP2012-189977A, good performance such as high sensitivity is obtained. However, there is still room for improvement in the occurrence of scum particularly in a fine pattern.

Accordingly, it is an object of the present invention to provide an actinic ray-sensitive or radiation-sensitive resin composition having high preservation stability, allowing a good pattern shape to be obtained when a fine pattern (particularly having a line width or space width of 50 nm or less) is formed, and capable of significantly reducing the occurrence of scum. It is another object of the invention to provide an actinic ray-sensitive or radiation-sensitive film using the actinic ray-sensitive or radiation-sensitive resin composition, a pattern forming method, a method for manufacturing an electronic device, and a compound.

The inventors have found that the above objects can be achieved by the following aspects.

[1] An actinic ray-sensitive or radiation-sensitive resin composition including a compound (I) that generates an acid upon irradiation with actinic rays or radiation and has an ionic structure and a zwitterionic structure.

[2] The actinic ray-sensitive or radiation-sensitive resin composition according to [1], wherein the compound (I) is a compound including at least two anionic groups and at least one cationic group that are linked via covalent bonding.

[3] The actinic ray-sensitive or radiation-sensitive resin composition according to [1] or [2], wherein the compound (I) is a compound including two anionic groups and one cationic group that are linked via covalent bonding.

[4] The actinic ray-sensitive or radiation-sensitive resin composition according to any one of [1] to [3], wherein the ionic structure and the zwitterionic structure are each independently a structure including an acid anionic group, and

wherein the compound (I) generates a plurality of acid groups having different pKa values upon irradiation with the actinic rays or radiation.

[5] The actinic ray-sensitive or radiation-sensitive resin composition according to any one of [1] to [4], wherein the compound (I) is a compound represented by any of the following general formulas (I)-1 to (I)-3:

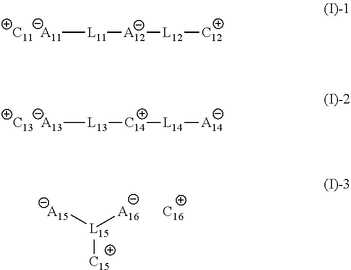

In general formulas (I)-1 to (I)-3,
A₁₁⁻ to A₁₆⁻ each independently represent an acid anionic group.
C₁₁⁺ to C₁₆⁺ each independently represent a cationic group.
L₁₁ to L₁₄ each independently represent a divalent organic group.
L₁₅ represents a trivalent organic group.

[6] The actinic ray-sensitive or radiation-sensitive resin composition according to [5], wherein, in a compound PI-1 formed by replacing a counter cation of the acid anionic group represented by A₁₁⁻ in the compound represented by general formula (I)-1 with H⁺ and adding H⁺ to the acid anionic group represented by A₁₂⁻, a pKa of a group represented by A₁₁H is smaller than a pKa of a group represented by A₁₂H,
wherein, in a compound PI-2 formed by replacing a counter cation of the acid anionic group represented by A₁₃⁻ in the compound represented by general formula (I)-2 with H⁺ and adding H⁺ to the acid anionic group represented by A₁₄⁻, a pKa of a group represented by A₁₃H is smaller than a pKa of a group represented by A₁₄H, and
wherein, in a compound PI-3 formed by adding H⁺ to the acid anionic group represented by A₁₅⁻ in the compound represented by general formula (I)-3 and replacing a counter cation of the acid anionic group represented by A₁₆⁻ with H⁺, a pKa of a group represented by A₁₅H is smaller than a pKa of a group represented by A₁₆H.

[7] The actinic ray-sensitive or radiation-sensitive resin composition according to [5] or [6], wherein A₁₁⁻ and A₁₃⁻ to A₁₆⁻ in formulas (I)-1 to (I)-3 each independently represent an acid anionic group represented by the following formula (A-1) or (A-2):

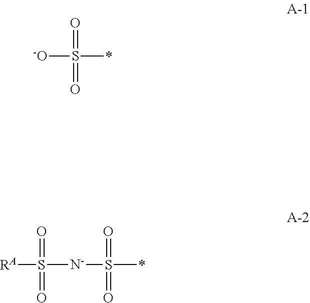

In formulas (A-1) to (A-2),
R^{A} represents an organic group.
* represents a bonding position.

[8] The actinic ray-sensitive or radiation-sensitive resin composition according to any one of [5] to [7], wherein A₁₂⁻ in formula (I)-1 represents an acid anionic group represented by any of the following formulas (B-1) to (B-3):

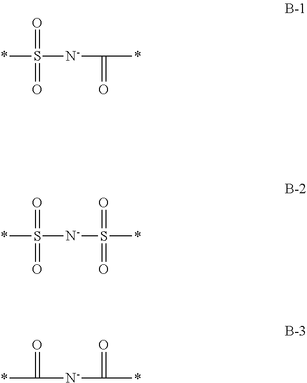 [SMILES: *C(=O)[N-]S(*)(=O)=O ; *S(=O)(=O)[N-]S(*)(=O)=O ; *C(=O)[N-]C(*)=O]

In formula (B-1) to (B-3),
* represents a bonding position.

[9] An actinic ray-sensitive or radiation-sensitive film formed using the actinic ray-sensitive or radiation-sensitive resin composition according to any one of [1] to [8].

[10] A pattern forming method including the steps of:
forming an actinic ray-sensitive or radiation-sensitive film on a substrate using the actinic ray-sensitive or radiation-sensitive resin composition according to any one of [1] to [8];
exposing the actinic ray-sensitive or radiation-sensitive film to light; and
developing the exposed actinic ray-sensitive or radiation-sensitive film using a developer.

[11] A method for manufacturing an electronic device, the method including the pattern forming method according to [10].

[12] A compound represented by any of the following general formulas (IA)-1 to (IA)-3:

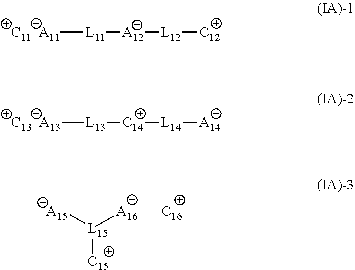

In general formulas (IA)-1 to (IA)-3,
A₁₁⁻ and A₁₃⁻ to A₁₆⁻ each independently represent an acid anionic group represented by formula (A-1) or (A-2) below.
A₁₂⁻ represents an acid anionic group represented by any of formulas (B-1) to (B-3) below.
C₁₁⁺ to C₁₆⁺ each independently represent a cationic group.
L₁₁ to L₁₄ each independently represent a divalent organic group.
L₁₅ represents a trivalent organic group.

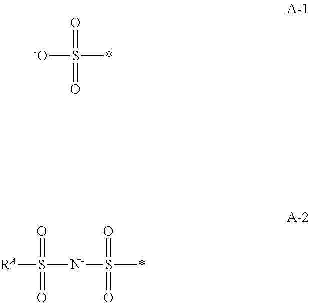

In formulas (A-1) to (A-2),
R^{A} represents an organic group.
* represents a bonding position.

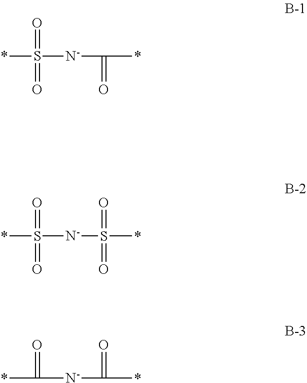 [SMILES: *C(=O)[N-]S(*)(=O)=O ; *S(=O)(=O)[N-]S(*)(=O)=O ; *C(=O)[N-]C(*)=O]

In formulas (B-1) to (B-3),
* represents a bonding position.

The present invention can provide an actinic ray-sensitive or radiation-sensitive resin composition having high preservation stability, allowing a good pattern shape to be obtained when a fine pattern (particularly having a line width or space width of 50 nm or less) is formed, and capable of significantly reducing the occurrence of scum. The present invention can also provide an actinic ray-sensitive or radiation-sensitive film using the actinic ray-sensitive or radiation-sensitive resin composition, a pattern forming method, a method for manufacturing an electronic device, and a compound.

### DESCRIPTION OF THE PREFERRED EMBODIMENTS

The present invention will next be described in detail.

Description of structural requirements described below may be made on the basis of representative embodiments of the present invention. However, the invention is not limited to theses embodiments.

As for notations of groups (atomic groups) in the present specification, a notation that is not specified as substituted and unsubstituted is intended to encompass groups having no substituent and groups having a substituent, so long as the notation does not depart from the spirit of the invention. For example, an “alkyl group” is intended to encompass not only an alkyl group having no substituent (an unsubstituted alkyl group) but also an alkyl group having a substituent (a substituted alkyl group). In the present specification, an “organic group” is a group including at least one carbon atom.

Preferably, the substituent is a monovalent substituent, unless otherwise specified.

In the present specification, “actinic rays” or “radiation” means, for example, an emission line spectrum of a mercury lamp, far-ultraviolet rays typified by excimer laser light, extreme ultraviolet light (EUV light), X-rays, an electron beam (EB), etc.

In the present specification, “light” means actinic rays or radiation.

In the present specification, “exposure to light” is intended to encompass not only exposure to an emission line spectrum of a mercury lamp, far-ultraviolet rays typified by an excimer laser light, X-rays, EUV light, etc. but also image drawing using an electron beam or a particle beam such as an ion beam.

In the present specification, “to” is used to mean that numerical values before and after the “to” are used as the lower limit and the upper limit.

In the present specification, no limitation is imposed on the bonding direction of a divalent group, unless otherwise specified. For example, when Y in a compound represented by formula “X—Y—Z” is —COO—, Y may be —CO—O— or may be —O—CO—. This compound may be “X—CO—O—Z” or may be “X—O—CO—Z.”

In the present specification, (meth)acrylate is intended to refer to acrylate and methacrylate, and (meth)acrylic is intended to refer to acrylic and methacrylic.

In the present specification, the weight average molecular weight (Mw), number average molecular weight (Mn), and dispersity (hereinafter may be referred to also as the “molecular weight distribution”) (Mw/Mn) of a compound are defined as polystyrene-equivalent values determined by GPC (Gel Permeation Chromatography) measurement (solvent: tetrahydrofuran, flow rate (injection amount of a sample): 10 μL, columns: TSK gel Multipore HXL-M manufactured by TOSOH Corporation, column temperature: 40° C., flow velocity: 1.0 mUminute, detector: differential refractive index detector) using a GPC apparatus (HLC-8120GPC manufactured by TOSOH Corporation).

In the present specification, the acid dissociation constant (pKa) is the pKa in an aqueous solution and is specifically a value determined by computation using the following software package 1 based on a Hammett substituent constant and a database of known literature values.

All pKa values in the present specification are values determined by computation using this software package.

Software package 1: Advanced Chemistry Development (ACD/Labs) Software V 8.14 for Solaris (1994-2007 ACD/Labs).

The pKa can also be determined by a molecular orbital calculation method. In one specific example of this method, H⁺ dissociation free energy in an aqueous solution is computed based on a thermodynamic cycle to compute the pKa. As for the method for computing the H⁺ dissociation free energy, the density functional theory (DFT), for example, can be used for the computation. Various other methods have been reported in literature etc., but the computation method is not limited thereto. There are a plurality of software applications capable of performing the DFT, and one example is Gaussian 16.

In the present specification, the pKa is a value determined by computation using the software package 1 based on the Hammett substituent constant and the database of known literature values as described above. When the pKa cannot be computed using this method, a value obtained using Gaussian 16 based on the DFT (density functional theory) is used.

In the present specification, the pKa is a “value in an aqueous solution” as described above. When the pKa in an aqueous solution cannot be computed, the “pKa in a dimethyl sulfoxide (DMSO) solution” is used.

“Solids” are components forming an actinic ray-sensitive or radiation-sensitive film, and a solvent is not included. Any component included in the actinic ray-sensitive or radiation-sensitive film is considered as a solid component even when the component is in a liquid form.

In the present specification, when the phrase “optionally having a substituent” or the phrase “may have a substituent” appears, no particular limitation is imposed on the type of substituent, the position of the substituent, and the number of substituents. The number of substituents may be, for example, 1, 2, 3, or more. Examples of the substituent include monovalent nonmetallic atomic groups except for a hydrogen atom, and the substituent can be selected from, for example, the following substituents T.

### (Substituents T)

Examples of the substituents T include: halogen atoms such as a fluorine atom, a chlorine atom, a bromine atom, and an iodine atom; alkoxy groups such as a methoxy group, an ethoxy group, and a tert-butoxy group; aryloxy groups such as a phenoxy group and a p-tolyloxy group; alkoxycarbonyl groups such as a methoxycarbonyl group, a butoxycarbonyl group, and a phenoxycarbonyl group; acyloxy groups such as an acetoxy group, a propionyloxy group, and a benzoyloxy group; acyl groups such as an acetyl group, a benzoyl group, an isobutyryl group, an acryloyl group, a methacryloyl group, and a methoxalyl group; alkylsulfanyl groups such as a methylsulfanyl group and a tert-butylsulfanyl group; arylsulfanyl groups such as a phenylsulfanyl group and a p-tolylsulfanyl group; alkyl groups; alkenyl groups; cycloalkyl groups; aryl groups; heteroaryl groups; a hydroxy group; a carboxy group; a formyl group; a sulfo group; a cyano group; alkylaminocarbonyl groups; arylaminocarbonyl groups; sulfonamido groups; silyl groups; an amino group; monoalkylamino groups; dialkylamino groups; arylamino groups; and combinations thereof.

### [Actinic Ray-Sensitive or Radiation-Sensitive Resin Composition]

The actinic ray-sensitive or radiation-sensitive resin composition according to the present invention (hereinafter may be referred to also as the “composition of the invention”) is

an actinic ray-sensitive or radiation-sensitive resin composition including a compound (I) that generates an acid upon irradiation with actinic rays or radiation and that has an ionic structure and a zwitterionic structure.

In the present specification, the term “ionic structure” means a structure in which a pair of a “positively charged functional group (cationic group)” and a “negatively charged functional group (anionic group)” form an ion pair via ionic bonding (with no covalent bonding). In the present specification, the ionic structure is referred to also as a “free ionic structure” in order to avoid confusion with the “zwitterionic structure.”

The term “zwitterionic structure” means a structure in which a pair of a “positively charged functional group (cationic group)” and a “negatively charged functional group (anionic group)” are linked via covalent bonding.

The phrase “via covalent bonding” is intended to encompass both a mode in which the cationic group and the anionic group are bonded via a single bond and a mode in which the cationic group and the anionic group are bonded via a linking group.

The present invention has the structure described above. Therefore, high preservation stability is obtained, and a good pattern shape is obtained when a fine pattern (particularly having a line width or space width of 50 nm or less) is formed). Moreover, the occurrence of scum can be reduced significantly.

The reason for this is unclear but may be as follows.

As described later, the compound (I) included in the composition of the invention can function as a compound that generates an acid necessary for the reaction of a resin in exposed portions and as an acid diffusion control agent that traps an excess portion of the acid generated in the exposed portions. As described above, the compound (I) has the zwitterionic structure in which a pair of cationic and anionic groups are linked via covalent bonding. Therefore, excessive migration of the acid generated from the compound (I) in exposed portions of a film and excessive migration of the compound (I) in unexposed portions of the film (in particular, excessive migration between the exposed portions and unexposed portions) tend to be prevented as compared to the case in which no zwitterionic structure is present. In this case, the desired reaction may proceed in the exposed portions with high precision. As a result, a good pattern shape is obtained even when a fine pattern (particularly having a line width or space width of 50 nm or less) is formed.

As described above, the compound (I) has, in addition to the zwitterionic structure, the free ionic structure that is a structure in which a pair of cationic and anionic groups form an ion pair via ionic bonding (with no covalent bonding). With this structure, the interaction between the cationic group in the zwitterionic structure and the anionic group in the free ionic structure and the interaction between the anionic group in the zwitterionic structure and the cationic group in the free ionic structure can easily occur in the composition, and this may allow cationic groups etc. susceptible to nucleophilic attack to be present stably in the compound (I). It is therefore inferred that the compound (I) is unlikely to decompose during storage of the composition and the composition has high preservation stability.

In a compound having a zwitterionic structure, a pair of cationic and anionic groups are linked via covalent bonding. Therefore, aggregation of the compound may occur, so that scum easily occurs during development with an alkali developer (alkali development) and during development with an organic solvent developer (organic solvent development). However, since the compound (I) in the invention has the free ionic structure in addition to the zwitterionic structure, aggregation of molecules is prevented, and the solubility of the compound (I) in the alkali developer and in the organic solvent developer is high, so that the occurrence of scum can be reduced significantly.

The composition of the invention is preferably a resist composition and may be a positive-type resist composition or a negative-type resist composition. The composition of the invention may be a resist composition for alkali development or a resist composition for organic solvent development.

The composition of the invention is preferably a chemical amplification-type resist composition and more preferably a chemical amplification positive-type resist composition.

The resist composition is typically a chemical amplification-type resist composition.

In the following description, the components of the composition of the invention will be described in detail.

<(I) Compound that Generates Acid Upon Irradiation with Actinic Rays or Radiation and has Ionic Structure and Zwitterionic Structure>

As described above, the actinic ray-sensitive or radiation-sensitive resin composition of the invention includes the compound (I) that generates an acid upon irradiation with actinic rays or radiation and has the free ionic structure and the zwitterionic structure (the compound is referred to simply as the “compound (I)”).

The free ionic structure is a structure in which a pair of cationic and anionic groups form an ion pair via ionic bonding (with no covalent bonding) as described above and is preferably a structure in which a pair of a cationic group and an acid anionic group form an ion pair via ionic bonding.

The zwitterionic structure is a structure in which a pair of cationic and anionic groups are linked via covalent bonding as described above and is preferably a structure in which a pair of a cationic group and an acid anionic group are linked via covalent bonding.

In other words, the free ionic structure and the zwitterionic structure are each preferably a structure including an acid anionic group.

The phrase “the compound (I) has the free ionic structure and the zwitterionic structure” means that the compound (I) has the free ionic structure and the zwitterionic structure separately, and the cationic group and anionic group in the free ionic structure are not the same as the cationic group and anionic group in the zwitterionic structure. Specifically, the cationic group in the free ionic structure does not serve also as the cationic group in the zwitterionic structure, and the anionic group in the free ionic structure does not serve also as the anionic group in the zwitterionic structure.

Therefore, in the compound (I), at least one of the cationic group or the anionic group in the free ionic structure is substantially linked to the zwitterionic structure via covalent bonding.

The compound (I) may have one free ionic structure or a plurality of free ionic structures. When the compound (I) has a plurality of free ionic structures, the plurality of free ionic structures may be the same or different.

The compound (I) may have one zwitterionic structure or a plurality of zwitterionic structures. When the compound (I) has a plurality of zwitterionic structures, the plurality of zwitterionic structures may be the same or different.

The compound (I) is a compound (photoacid generator) that generates an acid upon irradiation with actinic rays or radiation.

The compound (I) is preferably a compound that generates an acid group corresponding the acid anionic group in the free ionic structure and an acid group corresponding to the acid anionic group in the zwitterionic structure upon irradiation with actinic rays or radiation.

In the compound (I), the cationic group in the free ionic structure may be linked to the zwitterionic structure via covalent bonding, or the anionic group in the free ionic structure may be linked to the zwitterionic structure via covalent bonding.

As described above, the anionic group in the free ionic structure is preferably an acid anionic group, and specific examples of the anionic group include an organic group including an acid anionic group represented by formula (A-1) or (A-2) described later and an organic group including an acid anionic group represented by any of formulas (B-1) to (B-3) described later.

The acid anionic group may be an acid anionic group represented by formula (A-1) or (A-2) described later or an acid anionic group represented by any of formulas formula (B-1) to (B-3) described later.

No particular limitation is imposed on the cationic group in the free ionic structure.

The cationic group is typically an organic cationic group and is preferably a group having a sulfonium cation or an iodonium cation.

Examples of the cationic group include a cation represented by formula (ZaI) described later, a cation represented by formula (ZaII) described later, a group represented by formula (ZBI) described later, a group represented by formula (ZBII) described later, *—S⁺(R⁴⁰¹)—*, and *—I⁺—*. * represents a bonding position. R⁴⁰¹ will be described later.

As described above, the anionic group in the zwitterionic structure is preferably an acid anionic group, and specific examples of the anionic group include an organic group including an acid anionic group represented by formula (A-1) or (A-2) below and an organic group including an acid anionic group represented by any of formulas (B-1) to (B-3) below.

The acid anionic group may be an acid anionic group represented by formula (A-1) or (A-2) below or an acid anionic group represented by any of formulas (B-1) to (B-3) below.

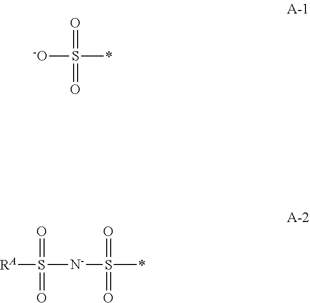

In formulas (A-1) to (A-2) above,
R^{A} represents an organic group.
* represents a bonding position.

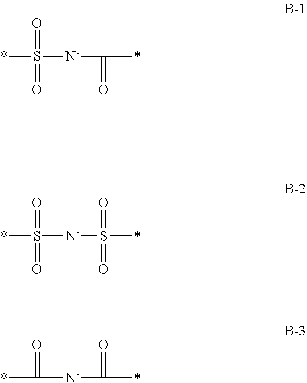 [SMILES: *C(=O)[N-]S(*)(=O)=O ; *S(=O)(=O)[N-]S(*)(=O)=O ; *C(=O)[N-]C(*)=O]

In formulas (B-1) to (B-3) above,
* represents a bonding position.

No particular limitation is imposed on the organic group represented by R^{A}, and the organic group is, for example, an organic group having 1 to 30 carbon atoms. No particular limitation is imposed on the organic group, and the organic group is preferably an alkyl group, a cycloalkyl group, or an aryl group.

No particular limitation is imposed on the alkyl group, and the alkyl group may be linear or branched. The alkyl group is preferably an alkyl group having 1 to 15 carbon atoms and more preferably an alkyl group having 1 to 10 carbon atoms.

No particular limitation is imposed on the cycloalkyl group, and the cycloalkyl group may be monocyclic and may be polycyclic. The cycloalkyl group is preferably a cycloalkyl group having 3 to 15 carbon atoms and more preferably a cycloalkyl group having 3 to 10 carbon atoms.

No particular limitation is imposed on the aryl group. The aryl group is preferably an aryl group having 6 to 20 carbon atoms and more preferably an aryl group having 6 to 10 carbon atoms.

The above alkyl, cycloalkyl, and aryl groups may each have a substituent. No particular limitation is imposed on the substituent, and examples of the substituent include the substituents T described above. In particular, the substituent is preferably a fluorine atom or a cyano group.

No particular limitation is imposed on the cationic group in the zwitterionic structure. The cationic group is preferably an organic cationic group and is preferably a group having a sulfonium cation or an iodonium cation.

Examples of the cationic group include the cation represented by formula (ZaI) described later, the cation represented by formula (ZaII) described later, the group represented by formula (ZBI) described later, the group represented by formula (ZBII) described later, *—S⁺(R⁴⁰¹)—* and *—I⁺—*. * represents a bonding position. R⁴⁰¹ will be described later.

The compound (I) is preferably a compound including at least two anionic groups and at least one cationic group that are linked via covalent bonding.

Each cationic group of the “at least one cationic group” is the cationic group in a free ionic structure or the cationic group in a zwitterionic structure.

At least one of the “at least one cationic group” is the cationic group in a zwitterionic structure.

Each anionic group of the “at least two anionic groups” is the anionic group in a free ionic structure or the anionic group in a zwitterionic structure.

At least one of the “at least two anionic groups” is the anionic group in a zwitterionic structure.

Preferably, the “at least two anionic groups” include an acid anionic group.

When the compound (I) is a compound including at least two anionic groups and at least one cationic group that are linked via covalent bonding, no particular limitation is imposed on the number of cationic groups. The number of cationic groups is preferably 4 or less and more preferably 2 or less.

When the compound (I) is a compound including at least two anionic groups and at least one cationic group that are linked via covalent bonding, no particular limitation is imposed on the number of anionic groups. The number of anionic groups is preferably 4 or less and more preferably 3 or less.

The compound (I) is preferably a compound including two anionic groups and one cationic group that are linked via covalent bonding.

This compound is a compound having one free ionic structure and one zwitterionic structure.

Preferably, each anionic group of the “two anionic groups” is an acid anionic group.

It is preferable that the ionic structure and the zwitterionic structure are each a structure including an acid anionic group and

that the compound (I) generates a plurality of acid groups having different pKa values upon irradiation with actinic rays or radiation.

The compound (I) generates a plurality of acid groups having different pKa values upon irradiation with actinic rays or radiation, and the resulting compound (PI) has an acid group having a higher acid strength (acid group 1) and an acid group having a lower acid strength (acid group 2) that are present in the same compound. The acid group 1 reacts with an acid-decomposable group in a resin described later, and the acid group 2 captures an excess portion of the acid generated in the exposed portions to prevent diffusion of the acid to the unexposed portions. Therefore, the use of the compound (I) is preferred because a good pattern shape can be obtained.

The pKa values of the plurality of acid groups generated from the compound (I) upon irradiation with actinic rays or radiation are determined using the following method.

(1) Suppose that all the acid anionic groups included in the compound (I) are replaced with their corresponding acid groups to generate an acid group-containing compound (PIA). In this case, there are two cases depending on the structure of the compound (I). In one case (case A), the acid group-containing compound (PIA) generated is a single compound (single molecule) having a plurality of acid groups. In the other case (case B), a plurality of compounds (plurality of molecules) each having at least one acid group are formed.

(2) The case A (its one specific example is mode 1 described below) will be described. A compound in which, among the plurality of acid groups included in the acid group-containing compound (PIA), an acid group having the smallest acid dissociation constant is reconverted to the corresponding acid anionic group is defined as an acid group-containing compound (PIA-1). The pKa when the acid group-containing compound (PIA) is converted to the acid group-containing compound (PIA-1) is determined and used as the pKa of the acid group reconverted to the acid anionic group. Next, a compound in which, among one or the plurality of acid groups included in the acid group-containing compound (PIA-1), an acid group having the smallest acid dissociation constant (when only one acid group is present, this acid group is used) is reconverted to the corresponding acid anionic group is defined as an acid group-containing compound (PIA-2). The pKa when the acid group-containing compound (PIA-1) is converted to the acid group-containing compound (PIA-2) is determined and used as the pKa of the acid group reconverted to the acid anionic group. This procedure is repeated until the resulting compound has no acid group, and the pKa values of the plurality of acid groups included in the acid group-containing compound (PIA) are thereby determined.

In the above method, when the number of acid groups having the smallest acid dissociation constant among the plurality of acid groups is two or more, one of these acid groups is arbitrarily selected, and the pKa when the compound is converted to a compound (PIA #) obtained by reconverting the selected acid group to its corresponding acid anionic group is determined. As for the pKa value of each of the remaining (unselected) acid groups, the pKa when the compound (PIA #) is converted to a “compound (PIA ##) obtained by reconverting an acid group newly selected from the remaining acid groups to its corresponding acid anionic group” is determined and used.

(3) In the case B (its one specific example is mode 2 described below), a plurality of compounds (a plurality of molecules) each having at least one acid group are present, and the pKa of each acid group in each compound is determined.

For a compound having one acid group, the pKa when the acid group in this compound is reconverted to the corresponding acid anionic group is used as the pKa of the acid group. When the compound having at least one acid group is a compound having a plurality of acid groups, the pKa of each acid group is determined using the method described in (2) above.

(4) When the acid group-containing compound (PIA) includes an iodine cation (I⁺) as a constituent element of the cationic group, a form (I⁺H) obtained by adding a hydrogen atom to the iodine cation (I⁺) is used as the acid group-containing compound (PIA), and (2) and (3) described above are performed.

In the mode 1, the mode 2, and Examples described later, the pKa values of the plurality of acid groups in the compound (I) are determined, and the smallest acid dissociation constant (pKa) is selected and denoted as an acid dissociation constant a1 (pKa1). Next, the second smallest acid dissociation constant is selected and denoted as an acid dissociation constant a2 (pKa2), and the third smallest acid dissociation constant is selected and denoted as an acid dissociation constant a3 (pKa3). This procedure is repeated to determine acid dissociation constants one by one.

For a plurality of acid groups having the same pKa, one of them is selected, and an acid dissociation constant number is assigned to the pKa.

A method for measuring the pKa will be specifically described below. The acid dissociation constant a1 (first acid dissociation constant) is smaller than the acid dissociation constant a2 (second acid dissociation constant).

### (Mode 1)

A method for measuring the pKa values of two acid groups derived from a compound including one cationic group and two acid anionic groups and serving as the compound (I) will be described below.

The acid anionic group in the free ionic structure is denoted as A₁⁻, and the acid anionic group in the zwitterionic structure is denoted as A₂ (the pKa of the acid group (A₁H) derived from A₁⁻ (the acid dissociation constant a1) <the pKa of the acid group (A₂H) derived from A₂ (the acid dissociation constant a2).

In a compound (PIA) obtained by replacing a counter cation of the acid anionic group represented by A₁⁻ with H⁺ and adding H⁺ to the acid anionic group represented by A₂, the pKa of the group represented by A₁H is smaller than the pKa of the group represented by A₂H.

The acid dissociation constant a1 and the acid dissociation constant a2 are determined by the method described above.

When an iodine cation (I⁺) is present in the cationic group, the acid dissociation constant is determined with a hydrogen atom added to the iodine cation, i.e., I⁺H.

When the acid dissociation constants of the compound (PIA) are determined, the pKa when the compound (PIA) (the compound PIA corresponds to a “compound having HA₁ and HA₂”) is converted to a “compound having A₁⁻ and HA₂” is the acid dissociation constant a1, and the pKa when the “compound having A₁⁻ and HA₂” is converted to a “compound having A₁ and A₂” is the acid dissociation constant a2.

The compound (PIA) corresponds to the acid generated when the compound (I) is irradiated with actinic rays or radiation.

In the above description, the pKa of the acid group (A₁H) derived from A₁⁻ (the acid dissociation constant a1) <the pKa of the acid group (A₂H) derived from A₂ (the acid dissociation constant a2). However, when the pKa of the acid group (A₁H) derived from A₁ (acid dissociation constant X)> the pKa of the acid group (A₂H) derived from A₂⁻ (acid dissociation constant Y), the acid dissociation constant X is the acid dissociation constant a2, and the acid dissociation constant Y is the acid dissociation constant a1.

When three or more acid anionic group structures are present, the acid dissociation constants can be determined one by one in the same manner as described above.

### (Mode 2)

A method for measuring the pKa values of two acid groups derived from a compound serving as the compound (I) and including two cationic groups and one acid anionic group linked via covalent bonding with one acid anionic group present as a free anion (not linked to a cationic group via covalent bonding) will be described below.

The acid anionic group serving as the free anion in the free ionic structure is denoted as A₁⁻, and the acid anionic group in the zwitterionic structure is denoted as A₂ (the pKa of the acid group (A₁H) derived from A₁⁻ is denoted as an (acid dissociation constant Y), and the pKa of the acid group (A₂H) derived from A₂ is denoted as an (acid dissociation constant X).

The acid dissociation constant X and the acid dissociation constant Y are determined by the method described above.

When an iodine cation (I⁺) is present in the cationic group in the zwitterionic structure, the acid dissociation constant is determined with a hydrogen atom added to the iodine cation, i.e., I⁺H.

When the acid dissociation constant of a compound (PIA) derived from the zwitterionic structure and obtained by adding H⁺ to the acid anionic group represented by A₂ is determined, the pKa when the compound (PIA) (the compound (PIA) corresponds to a “compound having HA₂”) is converted to a “compound having A₂” is the acid dissociation constant X.

When the acid dissociation constant of a compound (PIA) derived from an acid anionic group serving as the above-described free anion and obtained by adding H⁺ to the acid anionic group represented by A₁⁻ is determined, the pKa when the compound (PIA) (the compound PIA corresponds to a “compound having HA₁”) is converted to a “compound having A₁⁻” is the acid dissociation constant Y.

When the acid dissociation constant X and the acid dissociation constant Y are compared with each other, the acid dissociation constant X may be smaller than the acid dissociation constant Y. In this case, the acid dissociation constant X is the acid dissociation constant a1, and the acid dissociation constant Y is the acid dissociation constant a2.

In the mode 2, when a free acid anionic group is present, the acid dissociation constant of an acid (acid group) derived from the free acid anionic group and the acid dissociation constant of an acid group derived from the zwitterionic structure are measured. Then their magnitudes are compared to determine the acid dissociation constant a1 and the acid dissociation constant a2.

Even in a compound including an increased number of cationic groups and an increased number of acid anionic groups, the acid dissociation constants can be determined in the same manner as above.

In a compound (PIA) that is generated from the compound (I) in which the ionic structure and the zwitterionic structure have their respective acid anionic groups and which generates a plurality of acid groups having different pKa values upon irradiation with actinic rays or radiation, the difference between the acid dissociation constant (the acid dissociation constant a1) of the acid group (acid group A) having the highest acid strength and the acid dissociation constant (the acid dissociation constant a2) of the acid group having an acid strength that is the second highest next to that of the acid group A is preferably 0.70 or more and more preferably 1.60 or more. No particular limitation is imposed on the upper limit of the difference between the acid dissociation constant a1 and the acid dissociation constant a2, but the difference is, for example, 15.00 or less.

In the compound (PIA), no particular limitation is imposed on the acid dissociation constant a1, but the acid dissociation constant a1 is preferably −12.0 or more.

The compound (I) is preferable a compound represented by any of the following general formulas (I)-1 to (I)-3.

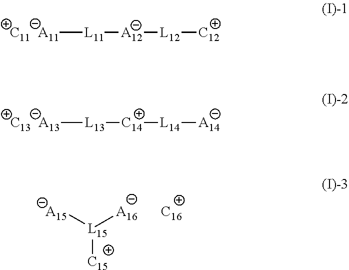

In general formulas (I)-1 to (I)-3,
A₁₁⁻ to A₁₆⁻ each independently represent an acid anionic group.
C₁₁⁺ to C₁₆⁺ each independently represent a cationic group.
L₁₁ to L₁₄ each independently represent a divalent organic group.
L₁₅ represents a trivalent organic group.

No particular limitation is imposed on the acid anionic group represented by each of A₁₁⁻ and A₁₃⁻ to A₁₆⁻. Examples of the acid anionic group include acid anionic groups represented by formulas (A-1) and (A-2) below.

Preferably, in formulas (I)-1 to (I)-3 above, A₁₁⁻ and A₁₃⁻ to A₁₆⁻ each independently represent an acid anionic group represented by the following formula (A-1) or (A-2).

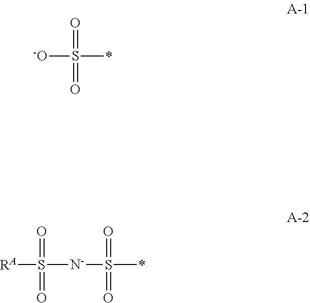

In general formulas (A-1) to (A-2) above,
R^{A} represents an organic group.
* represents a bonding position.

No particular limitation is imposed on the organic group represented by R^{A}, but examples of the organic group include organic groups having 1 to 30 carbon atoms. No particular limitation is imposed on the organic group, but examples thereof include alkyl groups, cycloalkyl groups, and aryl groups.

No particular limitation is imposed on the alkyl group, but the alkyl group may be linear or branched and is preferably an alkyl group having 1 to 15 carbon atoms and more preferably an alkyl group having 1 to 10 carbon atoms.

No particular limitation is imposed on the cycloalkyl group, but the cycloalkyl group may be monocyclic or polycyclic and is preferably a cycloalkyl group having 3 to 15 carbon atoms and more preferably a cycloalkyl group having 3 to 10 carbon atoms.

No particular limitation is imposed on the aryl group, and the aryl group is preferably an aryl group having 6 to 20 carbon atoms and more preferably an aryl group having 6 to 10 carbon atoms.

The alkyl, cycloalkyl, and aryl groups may each have a substituent. No particular limitation is imposed on the substituent, and examples of the substituent include the substituents T described above. In particular, the substituent is preferably a fluorine atom or a cyano group.

No particular limitation is imposed on the acid anionic group represented by A₁₂⁻, but examples of the acid anionic group include acid anionic groups represented by formulas (B-1) to (B-3) below.

In general formula (I)-1, A₁₂⁻ represents preferably an acid anionic group represented by any of the following formulas (B-1) to (B-3).

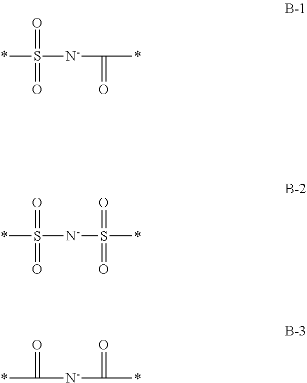

In general formulas (B-1) to (B-3) above,
* represents a bonding position.

No particular limitation is imposed on the cationic groups represented by C₁₁⁺, C₁₃⁺, and C₁₆⁺, but specific examples thereof include organic cations.

In particular, the organic cation is preferably a cation represented by formula (ZaI) (hereinafter referred to as a “cation (ZaI)”) or a cation represented by formula (ZaII) (hereinafter referred to as a “cation (ZaII)”)

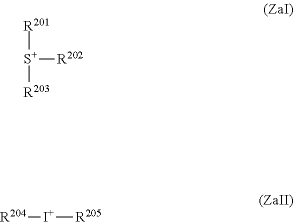

In formula (ZaI),

R²⁰¹, R²⁰², and R²⁰³ each independently represent an organic group.

The number of carbon atoms in each of the organic groups used as R²⁰¹, R²⁰², and R²⁰³ is preferably 1 to 30 and more preferably 1 to 20. Two selected from the group consisting of R²⁰¹ to R²⁰³ may be bonded together to form a ring structure, and the ring may include an oxygen atom, a sulfur atom, an ester group, an amido group, or a carbonyl group. Examples of the group formed from two selected from the group consisting of R²⁰¹ to R²⁰³ that are bonded together include alkylene groups (such as a butylene group and a pentylene group) and —CH₂—CH₂—O—CH₂—CH₂—.

Preferred examples of the form of the organic cation in formula (ZaI) include a cation (ZaI-1), a cation (ZaI-2), an organic cation (cation (ZaI-3b)) represented by formula (ZaI-3b), and an organic cation (cation (ZaI-4b)) represented by formula (ZaI-4b) that will be described later.

First, the cation (ZaI-1) will be described.

The cation (ZaI-1) is an arylsulfonium cation in which at least one of R²⁰¹, R²⁰², or R²⁰³ in formula (ZaI) is an aryl group.

In the arylsulfonium cation, each of R²⁰¹ to R²⁰³ may be an aryl group. Alternatively, some of R²⁰¹ to R²⁰³ may be an aryl group, and the rest may be an alkyl group or a cycloalkyl group.

Alternatively, one of R²⁰¹, R²⁰², or R²⁰³ may be an aryl group, and the remaining two of R²⁰¹ to R²⁰³ may be bonded together to form a ring structure. The ring may include an oxygen atom, a sulfur atom, an ester group, an amido group, or a carbonyl group. Examples of the group formed by bonding two selected from the group consisting of R²⁰¹ to R²⁰³ together include alkylene groups in which at least one methylene group is replaced by an oxygen atom, a sulfur atom, an ester group, an amido group, and/or a carbonyl group (such as a butylene group, a pentylene group, and a —CH₂—CH₂—O—CH₂—CH₂—).

Examples of the arylsulfonium cation include triarylsulfonium cations, diarylalkylsulfonium cations, aryldialkylsulfonium cations, diarylcycloalkylsulfonium cations, and aryldicycloalkylsulfonium cations.

Each aryl group included in the arylsulfonium cation is preferably a phenyl group or a naphthyl group and is more preferably a phenyl group. The aryl group may have a heterocyclic structure having an oxygen atom, a nitrogen atom, a sulfur atom, etc. Examples of the heterocyclic structure include a pyrrole residue, a furan residue, a thiophene residue, an indole residue, a benzofuran residue, and a benzothiophene residue. When the arylsulfonium cation has two or more aryl groups, the two or more aryl groups may be the same or different.

The alkyl group or the cycloalkyl group optionally included in the arylsulfonium cation is preferably a linear alkyl group having 1 to 15 carbon atoms, a branched alkyl group having 3 to 15 carbon atoms, or a cycloalkyl group having 3 to 15 carbon atoms and more preferably a methyl group, an ethyl group, a propyl group, a n-butyl group, a sec-butyl group, a t-butyl group, a cyclopropyl group, a cyclobutyl group, or a cyclohexyl group.

The aryl, alkyl, and cycloalkyl groups in R²⁰¹ to R²⁰³ may each have a substituent, and the substituent is preferably an alkyl group (having, for example, 1 to 15 carbon atoms), a cycloalkyl group (having, for example, 3 to 15 carbon atoms), an aryl group (having, for example, 6 to 14 carbon atoms), an alkoxy group (having, for example, 1 to 15 carbon atoms), a cycloalkylalkoxy group (having, for example, 1 to 15 carbon atoms), a halogen atom (for example, fluorine or iodine), a hydroxy group, a carboxy group, an ester group, a sulfinyl group, a sulfonyl group, an alkylthio group, or a phenylthio group.

Each substituent may have a substituent if possible. It is also preferable that the alkyl group has a halogen atom as a substituent and is therefore a halogenated alkyl group such as a trifluoromethyl group.

It is also preferable that any of these substituents are combined together to form an acid-decomposable group.

The acid-decomposable group means a group that is decomposed by the action of an acid to generate a polar group and is preferably a structure in which the polar group is protected by a leaving group that leaves by the action of an acid. The polar group and the leaving group will be described later.

Next, the cation (ZaI-2) will be described.

The cation (ZaI-2) is a cation in which R²⁰¹ to R²⁰³ in formula (ZaI) each independently represent an organic group having no aromatic ring. The aromatic ring is intended to encompass an aromatic ring including a heteroatom.

The number of carbon atoms in each of the organic groups having no aromatic ring and represented by R²⁰¹ to R²⁰³ is preferably 1 to 30 and more preferably 1 to 20.

R²⁰¹ to R²⁰³ are each independently preferably an alkyl group, a cycloalkyl group, an allyl group, or a vinyl group, more preferably a linear or branched 2-oxoalkyl group, a 2-oxocycloalkyl group, or an alkoxycarbonylmethyl group, and still more preferably a linear or branched 2-oxoalkyl group.

Examples of the alkyl and cycloalkyl groups in R²⁰¹ to R²⁰³ include: linear alkyl groups having 1 to 10 carbon atoms and branched alkyl groups having 3 to 10 carbon atoms (such as a methyl group, an ethyl group, a propyl group, a butyl group, and a pentyl group); and cycloalkyl groups having 3 to 10 carbon atoms (such as a cyclopentyl group, a cyclohexyl group, and a norbornyl group).

R²⁰¹ to R²⁰³ may each be further substituted with a halogen atom, an alkoxy group (having, for example, 1 to 5 carbon atoms), a hydroxy group, a cyano group, or a nitro group.

It is also preferable that the substituents in R²⁰¹ to R²⁰³ are each independently combined with another substituent to form an acid-decomposable group.

Next, the cation (ZaI-3b) will be described.

The cation (ZaI-3b) is a cation represented by the following formula (ZaI-3b).

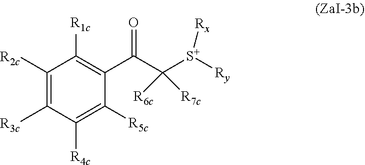

In formula (ZaI-3b), R_{1c} to R_{5c}, each independently represent a hydrogen atom, an alkyl group, a cycloalkyl group, an aryl group, an alkoxy group, an aryloxy group, an alkoxycarbonyl group, an alkylcarbonyloxy group, a cycloalkylcarbonyloxy group, a halogen atom, a hydroxy group, a nitro group, an alkylthio group, or an arylthio group.

R_{6c} and R_{7c} each independently represent a hydrogen atom, an alkyl group (such as a t-butyl group), a cycloalkyl group, a halogen atom, a cyano group, or an aryl group.

Rₓ and R_{y} each independently represent an alkyl group, a cycloalkyl group, a 2-oxoalkyl group, a 2-oxocycloalkyl group, an alkoxycarbonylalkyl group, an allyl group, or a vinyl group.

R_{1c} to R_{7c}, Rₓ, and R_{y} may each have a substituent, and it is also preferable that these substituents are each independently combined with another substituent to form an acid-decomposable group.

A combination of two or more selected from the group consisting of R_{1c} to R_{5c}, a pair of R_{5c} and R_{6c}, a pair of R_{6c} and R_{7c}, a pair of R^{5c} and Rₓ, and a pair of Rₓ and R_{y} may each be bonded together to form a ring. These rings may each independently include an oxygen atom, a sulfur atom, a ketone group, an ester bond, or an amide bond.

Each ring may be an aromatic or non-aromatic hydrocarbon ring, an aromatic or non-aromatic heterocyclic ring, or a polycyclic condensed ring formed by combining two or more of the above rings. The ring may be a 3- to 10-membered ring and is preferably a 4- to 8-membered ring and more preferably a 5- or 6-membered ring.

Examples of the groups formed by bonding two or more selected from the group consisting of R_{1c} to R_{5c}, bonding R_{6c} and R_{7c}, and bonding Rₓ and R_{y} include alkylene groups such as a butylene group and a pentylene group. A methylene group in the alkylene group may be replaced with a heteroatom such as an oxygen atom.

The group formed by bonding R^{5c} and R_{6c} and the group formed by bonding R^{5c} and Rₓ are each preferably a single bond or an alkylene group. Examples of the alkylene group include a methylene group and an ethylene group.

R_{1c} to R^{5c}, R_{6c}, R_{7c}, Rₓ, R_{y}, the ring formed by bonding together a combination of two or more selected from the group consisting of R_{1c} to R_{5c}, the ring formed by bonding together a pair of R^{5c} and R_{6c}, the ring formed by bonding together a pair of R_{6c} and R_{7c}, the ring formed by bonding together a pair of R^{5c} and Rₓ, and the ring formed by bonding together a pair of Rₓ and R_{y} may each have a substituent.

Next, the cation (ZaI-4b) will be described.

The cation (ZaI-4b) is a cation represented by the following formula (ZaI-4b).

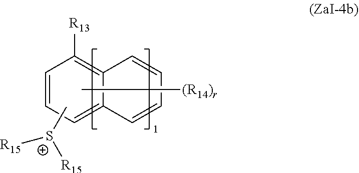

In formula (ZaI-4b), 1 represents an integer of from 0 to 2.

r represents an integer of from 0 to 8.

R₁₃ represents a hydrogen atom, a halogen atom (such as a fluorine atom or an iodine atom), a hydroxy group, an alkyl group, a halogenated alkyl group, an alkoxy group, a carboxy group, an alkoxycarbonyl group, or a group including a cycloalkyl group (a cycloalkyl group itself or a group including a cycloalkyl group as a part thereof). These groups may each have a substituent.

R₁₄ represents a hydroxy group, a halogen atom (such as a fluorine atom or an iodine atom), an alkyl group, a halogenated alkyl group, an alkoxy group, an alkoxycarbonyl group, an alkylcarbonyl group, an alkylsulfonyl group, a cycloalkylsulfonyl group, or a group including a cycloalkyl group (a cycloalkyl group itself or a group including a cycloalkyl group as a part thereof). These groups may each have a substituent. When a plurality of R¹⁴'s are present, they each independently represent any of the above groups such as a hydroxy group.

R¹⁵'s each independently represent an alkyl group, a cycloalkyl group, or a naphthyl group. The two R¹⁵'s may be bonded together to form a ring. When the two R¹⁵'s are bonded together to form a ring, the skeleton of the ring may include a heteroatom such as an oxygen atom or a nitrogen atom.

In one preferred mode, the two R¹⁵'s are each an alkylene group and are bonded together to form a ring structure. The above alkyl, cycloalkyl, and naphthyl groups and the ring formed by bonding the two R¹⁵'s may each have a substituent.

In formula (ZaI-4b), the alkyl group represented by each of R₁₃, R₁₄, and R₁₅s may be a linear or branched alkyl group. Preferably, the number of carbon atoms in the alkyl group is 1 to 10. Each alkyl group is preferably a methyl group, an ethyl group, a n-butyl group, a t-butyl group, etc.

It is also preferable that the substituents in R¹³ to R¹⁵'s, Rₓ, and R_{y} are each independently combined with another substituent to form an acid-decomposable group.

Next, formula (ZaII) will be described.

In formula (ZaII), R²⁰⁴ and R²⁰⁵ each independently represent an aryl group, an alkyl group, or a cycloalkyl group.

The aryl group represented by each of R²⁰⁴ and R²⁰⁵ is preferably a phenyl group or a naphthyl group and more preferably a phenyl group. The aryl group represented by each of R²⁰⁴ and R²⁰⁵ may be an aryl group having a heterocycle having an oxygen atom, a nitrogen atom, or a sulfur atom. Examples of the skeleton of the aryl group having a heterocycle include pyrrole, furan, thiophene, indole, benzofuran, and benzothiophene.

The alkyl or cycloalkyl group represented by each of R²⁰⁴ and R²⁰⁵ is preferably a linear alkyl group having 1 to 10 carbon atoms or a branched alkyl group having 3 to 10 carbon atoms (such as a methyl group, an ethyl group, a propyl group, a butyl group, or a pentyl group) or is a cycloalkyl group having 3 to 10 carbon atoms (such as a cyclopentyl group, a cyclohexyl group, or a norbornyl group).

The aryl, alkyl, and cycloalkyl groups represented by R²⁰⁴ and R²⁰⁵ may each independently have a substituent. Examples of the optional substituents in the aryl, alkyl, and cycloalkyl groups represented by R²⁰⁴ and R²⁰⁵ include alkyl groups (having, for example, 1 to 15 carbon atoms), cycloalkyl groups (having, for example, 3 to 15 carbon atoms), aryl groups (having, for example, 6 to 15 carbon atoms), alkoxy groups (having, for example, 1 to 15 carbon atoms), halogen atoms, a hydroxy group, and a phenylthio group. It is also preferable that the substituents in R²⁰⁴ and R²⁰⁵ are each independently combined with another substituent to form an acid-decomposable group.

Specific examples of the organic cation are shown below. However, the invention is not limited thereto.

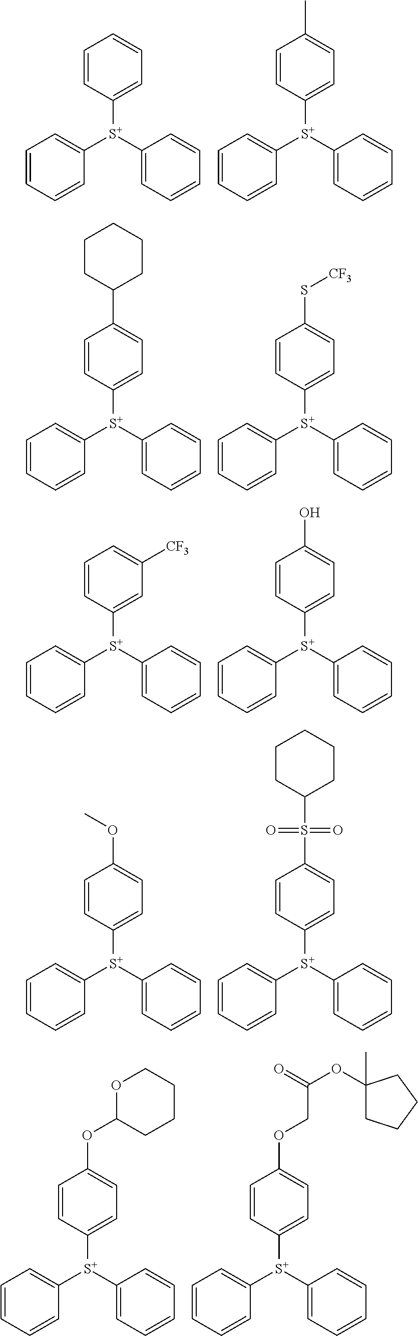

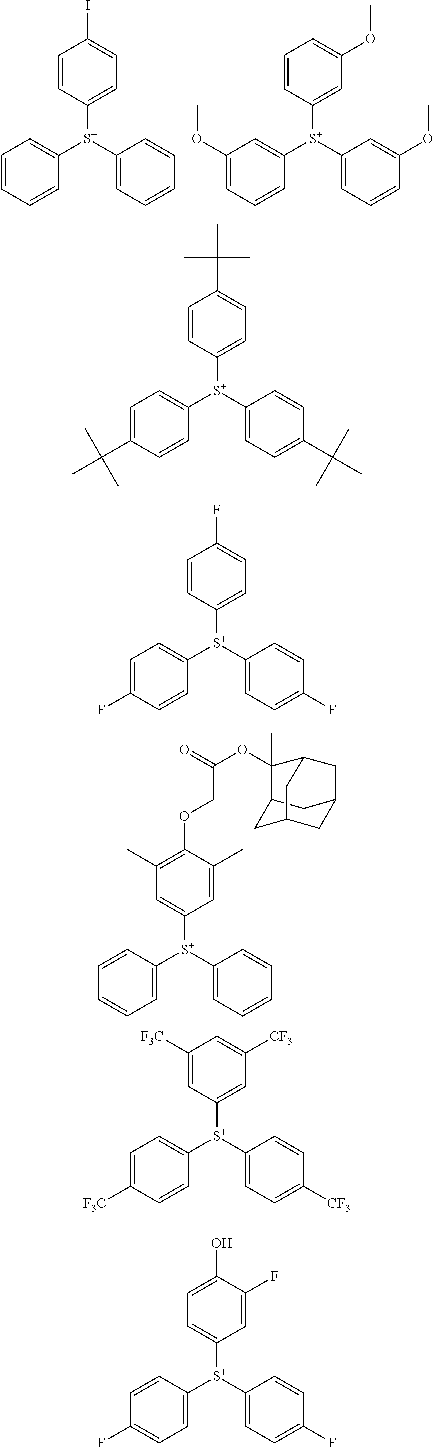

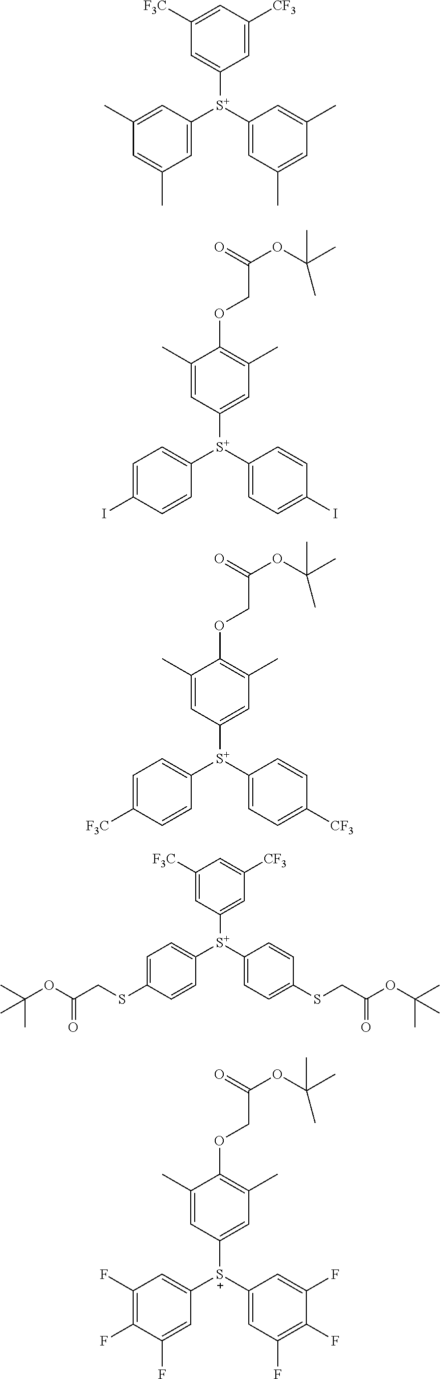

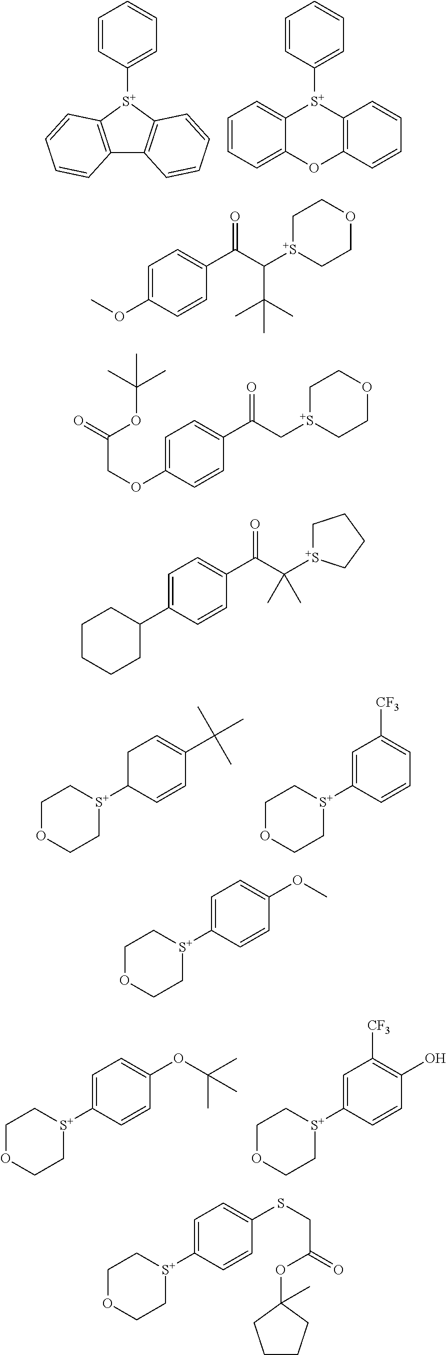

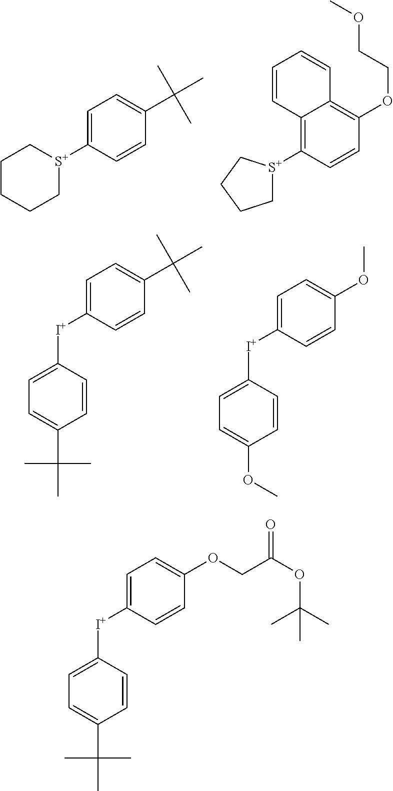 [SMILES: CC(C)(C)c1ccc([S+]2CCCCC2)cc1 ; COCCOc1ccc([S+]2CCCC2)c2ccccc12 ; CC(C)(C)c1ccc([I+]c2ccc(C(C)(C)C)cc2)cc1 ; COc1ccc([I+]c2ccc(OC)cc2)cc1 ; CC(C)(C)OC(=O)COc1ccc([I+]c2ccc(C(C)(C)C)cc2)cc1]

No particular limitation is imposed on the cationic groups represented by C₁₂⁺ and C₁₅⁺, but specific examples thereof include organic cationic groups.

In particular, the organic cationic group is preferably a group represented by formula (ZBI) or a group represented by formula (ZBII).

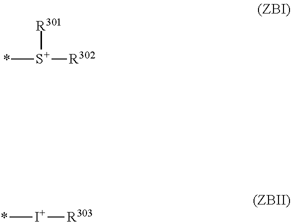

In formulas (ZBI) and (ZBII),

R³⁰¹, R³⁰², and R³⁰³ each independently represent an aryl group, an alkyl group, or a cycloalkyl group.

R³⁰¹ to R³⁰² may be bonded together to form a ring structure, and the ring may include an oxygen atom, a sulfur atom, an ester group, an amido group, or a carbonyl group.

* represents a bonding position.

The aryl group represented by each of R³⁰¹, R³⁰² and R³⁰³ is preferably a phenyl group or a naphthyl group and more preferably a phenyl group. The aryl group represented by each of R³⁰¹, R³⁰², and R³⁰³ may be an aryl group having a heterocycle having an oxygen atom, a nitrogen atom, a sulfur atom, etc. Examples of the skeleton of the aryl group having a heterocycle include pyrrole, furan, thiophene, indole, benzofuran, and benzothiophene.

The alkyl and cycloalkyl groups represented by R³⁰¹, R³⁰², and R³⁰³ are each preferably a linear alkyl group having 1 to 10 carbon atoms, a branched alkyl group having 3 to 10 carbon atoms (such as a methyl group, an ethyl group, a propyl group, a butyl group, or a pentyl group), or a cycloalkyl group having 3 to 10 carbon atoms (such as a cyclopentyl group, a cyclohexyl group, or a norbornyl group).

The aryl, alkyl, and cycloalkyl groups represented by R³⁰¹, R³⁰², and R³⁰³ may each have a substituent. Examples of the substituent that the aryl, alkyl, and cycloalkyl groups represented by R³⁰¹, R³⁰², and R³⁰³ may have include alkyl groups (having, for example, 1 to 15 carbon atoms), cycloalkyl groups (having, for example, 3 to 15 carbon atoms), aryl groups (having, for example, 6 to 15 carbon atoms), alkoxy groups (having, for example, 1 to 15 carbon atoms), halogen atoms, a hydroxy group, and a phenylthio group. It is also preferable that the substituents in R³⁰¹, R³⁰², and R³⁰³ are each independently combined with another substituent to form an acid-decomposable group. The acid-decomposable group is as described later.

R³⁰¹ to R³⁰² may be bonded together to form a ring structure, and the ring may include an oxygen atom, a sulfur atom, an ester group, an amido group, or a carbonyl group. Examples of the ring formed by bonding R³⁰¹ to R³⁰² together include alkylene groups (such as a butylene group and a pentylene group) and —CH₂—CH₂—O—CH₂—CH₂—.

No particular limitation is imposed on the cationic group represented by C₁₄+, but specific examples thereof include *—S⁺(R⁴⁰¹)—* and *—I⁺—*. * represents a bonding position.

R⁴⁰¹ represents an aryl group, an alkyl group. or a cycloalkyl group.

Specific examples of the aryl, alkyl, and cycloalkyl groups represented by R⁴⁰¹ include those for the aryl, alkyl, and cycloalkyl groups represented by R³⁰¹, R³⁰², and R³⁰³ and their preferred ranges are the same as those for the aryl, alkyl, and cycloalkyl groups represented by R³⁰¹, R³⁰², and R³⁰³.

The aryl, alkyl, and cycloalkyl groups represented by R⁴⁰¹ may each independently have a substituent. Examples of the substituent that the aryl, alkyl, and cycloalkyl groups represented by R⁴⁰¹ may have include alkyl groups (having, for example, 1 to 15 carbon atoms), cycloalkyl groups (having, for example, 3 to 15 carbon atoms), aryl groups (having, for example, 6 to 15 carbon atoms), alkoxy groups (having, for example, 1 to 15 carbon atoms), halogen atoms, a hydroxy group, and a phenylthio group. It is also preferable that substituents in R⁴⁰¹ are each independently combined with another substituent to form an acid-decomposable group. The acid-decomposable group is as described later.

No particular limitation is imposed on the divalent organic group represented by each of L₁₁ to L₁₄, and examples thereof include alkylene groups, cycloalkylene groups, aromatic ring groups, aromatic heterocyclic groups, —C(═O)—, —O—, —S(═O)₂—, —S—, and divalent linking groups formed by combining any of these groups.

No particular limitation is imposed on the alkylene group, but the alkylene group may be linear or branched and is preferably an alkylene group having 1 to 20 carbon atoms, more preferably an alkylene group having 1 to 10 carbon atoms, and still more preferably an alkylene group having 1 to 3 carbon atoms.

No particular limitation is imposed on the cycloalkylene group, but the cycloalkylene group is preferably a cycloalkylene group having 3 to 20 carbon atoms, more preferably a cycloalkylene group having 3 to 10 carbon atoms, and still more preferably a cycloalkylene group having 1 to 6 carbon atoms.

No particular limitation is imposed on the aromatic ring group, but the aromatic ring group may be monocyclic or polycyclic and is preferably an aromatic ring group having 6 to 20 carbon atoms, more preferably an aromatic ring group having 6 to 14 carbon atoms, and still more preferably an aromatic ring group having 6 to 10 carbon atoms.

No particular limitation is imposed on the aromatic heterocyclic group, but the aromatic heterocyclic group may be monocyclic or polycyclic. No particular limitation is imposed on the aromatic heterocycle included in the aromatic heterocyclic group, but examples of the aromatic heterocycle include thiophene, furan, pyrrole, benzothiophene, benzofuran, benzopyrrole, triazine, imidazole, benzimidazole, triazole, thiadiazole, and thiazole.

The alkylene, cycloalkylene, aromatic ring, and aromatic heterocyclic groups may each have a substituent. No particular limitation is imposed on the substituent, but examples of the substituent include the substituents T described above. The substituent is preferably a fluorine atom.

The divalent organic group is preferably an alkylene group, alkylene group-O—, an —O— alkylene group, alkylene group-C(═O)O—, alkylene group-O—C(═O)—, an alkylene group-O— alkylene group, or an aromatic ring group.

No particular limitation is imposed on the trivalent organic group represented by L₁₅, but examples thereof include a group formed by removing one hydrogen atom from a divalent organic group.

The divalent organic group is the same as the divalent organic group represented by any of L₁₁ to L₁₄ above, and its preferred range is also same as that of the divalent organic group represented by any of L₁₁ to L₁₄ above.

The compound represented by general formula (I)-1 above is a compound having one free ionic structure represented by C₁₁+A₁₁⁻ and one zwitterionic structure represented by A₁₂⁻-L₁₂-C₁₂+.

The compound represented by general formula (I)-2 above is a compound having one free ionic structure represented by C₁₃+A₁₃⁻ and one zwitterionic structure represented by C₁₄⁺-L₁₄-A₁₄⁻.

The compound represented by general formula (I)-3 above is a compound having one free ionic structure represented by A₁₆⁻C₁₆⁺ and one zwitterionic structure represented by A₁₅⁻-L₁₅-C₁₅⁺.

In a compound PI-1 obtained by replacing a counter cation of the acid anionic group represented by A₁₁⁻ in the compound represented by general formula (I)-1 with H⁺ and adding H⁺ to the acid anionic group represented by A₁₂, it is preferable that the pKa of the group represented by A₁₁H (corresponding to the acid dissociation constant a1 described above) is smaller than the pKa of the group represented by A₁₂H (corresponding to the acid dissociation constant a2 described above).

In a compound PI-2 obtained by replacing a counter cation of the acid anionic group represented by A₁₃⁻ in the compound represented by general formula (I)-2 above with H⁺ and adding H⁺ to the acid anionic group represented by A₁₄, it is preferable that the pKa of the group represented by A₁₃H (corresponding to the acid dissociation constant a1 described above) is smaller than the pKa of the group represented by A₁₄H (corresponding to the acid dissociation constant a2 described above).

In a compound PI-3 obtained by adding H⁺ to the acid anionic group represented by A₁₅ in the compound represented by general formula (I)-3 above and replacing a counter cation of the acid anionic group represented by A₁₆⁻ with H⁺, it is preferable that the pKa of the group represented by A₁₅H (corresponding to the acid dissociation constant a1 described above) is smaller than the PKa of the group represented by A₁₆H (corresponding to the acid dissociation constant a2 described above).

Specific examples of the compound (I) are shown below, but the invention is not limited thereto.

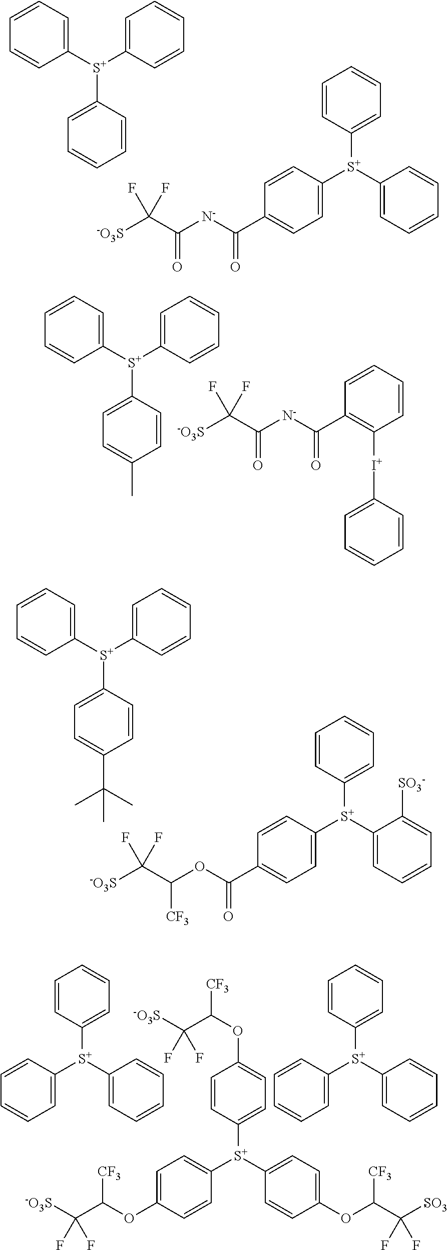

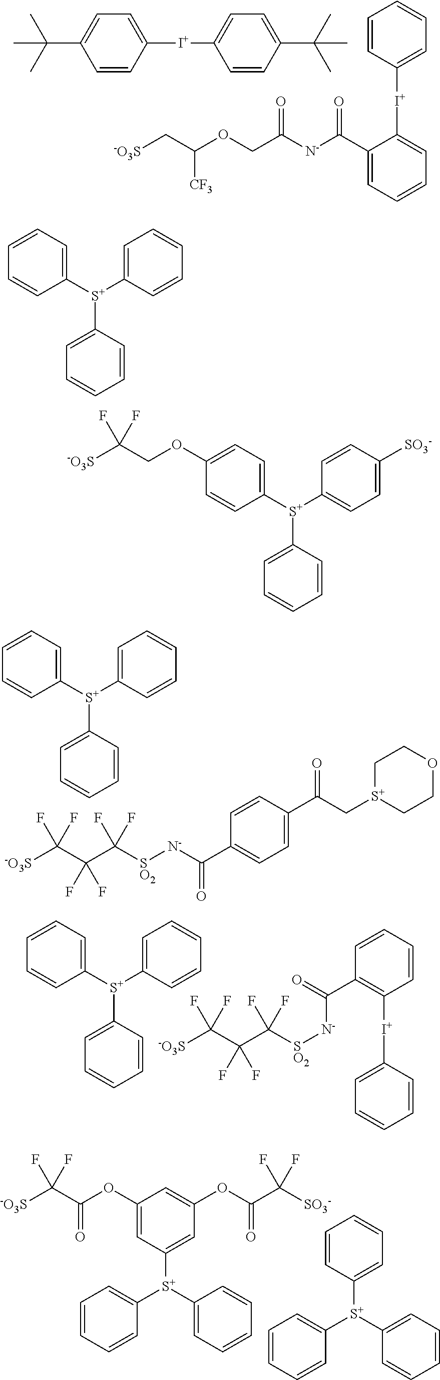

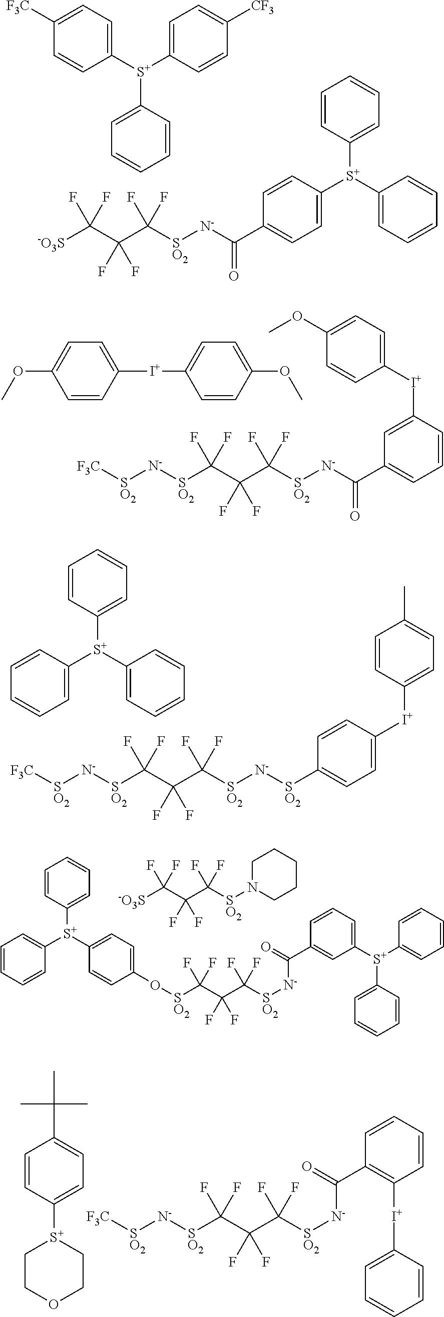

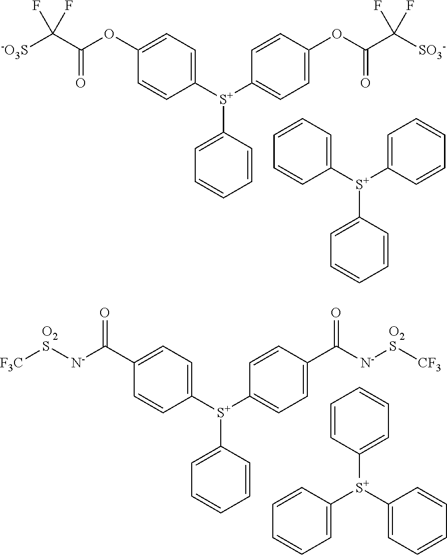

As described above, the compound (I) is a photoacid generator, has two anionic groups (preferably acid anionic groups), and can be used as a photoacid generator that generates an acid necessary for the reaction of the resin in exposed portions and also as an acid diffusion control agent.

When the compound (I) is used as a photoacid generator that generates an acid necessary for the reaction of the resin in the exposed portions and used in combination with a compound (CD) that can be used as an acid diffusion control agent described later, it is preferable that the acid generated from the compound (I) is stronger than the acid generated from the compound (CD).

When the compound (I) is used as an acid diffusion control agent, it is preferable that the compound (I) is used in combination with an photoacid generator that generates an acid necessary for the reaction of the resin in the exposed portions such that the acid generated from the photoacid generator is stronger than the acid generated from the compound (I).

When the compound (I) has a plurality of anionic groups and the acid dissociation constants of a plurality of acid groups generated upon irradiation with actinic rays or radiation differ from each other, the compound has a group serving as a strong acid (serving as a photoacid generator) and an acid weaker than the strong acid (serving as an acid diffusion control agent).

In this case, one compound can function as a photoacid generator and also as an acid diffusion control agent.

The compound (I) can be synthesized according to a well-known method. Specific examples of the synthesis of the compound represented by compound (I) will be shown later in Examples.

The molecular weight of the compound (I) is preferably 300 to 3000, more preferably 300 to 2000, and still more preferably 300 to 1500.

One compound (I) may be used alone, or two or more compounds (I) may be used in combination.

The content of the compound (I) in the composition of the invention (the total content when a plurality of compounds (I) are present) with respect to the total amount of solids in the composition is preferably 0.1 to 50% by mass, more preferably 0.5 to 30% by mass, still more preferably 5 to 25% by mass, and particularly preferably 5 to 20% by mass.

### <Resin (A)>

Preferably, the actinic ray-sensitive or radiation-sensitive resin composition (hereinafter may be referred to as the “composition”) includes a resin (A) that is decomposed by the action of an acid and thereby increased in polarity (hereinafter referred to as the “resin (A)”).

The resin (A) is typically an acid-decomposable resin, generally includes a group that is decomposed by the action of an acid and thereby increased in polarity (this group is hereafter referred to also as an “acid-decomposable group”), and preferably includes a repeating unit having the acid-decomposable group.

In the pattern forming method of the invention, when a developer used is an alkali developer, a preferred positive-type pattern is typically formed. When the developer used is an organic-based developer, a preferred negative-type pattern is typically formed.

The repeating unit having an acid-decomposable group is preferably a (repeating unit having an acid-decomposable group) described later and is also preferably a (repeating unit having an acid-decomposable group including an unsaturated bond).

### (Repeating Unit Having Acid-Decomposable Group)

The acid-decomposable group is a group that is decomposed by the action of an acid to generate a polar group. Preferably, the acid-decomposable group has a structure in which the polar group is protected by a leaving group that leaves by the action of an acid. Specifically, the resin (A) has a repeating unit having a group that is decomposed by the action of an acid to generate a polar group. The resin having this repeating unit is increased in polarity by the action of an acid. The degree of solubility in an alkali developer thereby increases, and the degree of solubility in an organic solvent decreases.

The polar group is preferably an alkali-soluble group, and examples thereof include: acidic groups such as a carboxy group, phenolic hydroxy groups, fluorinated alcohol groups, sulfonic acid groups, phosphoric acid groups, sulfonamido groups, sulfonylimido groups, (alkylsulfonyl)(alkylcarbonyl)methylene groups, (alkylsulfonyl)(alkylcarbonyl)imido groups, bis(alkylcarbonyl)methylene groups, bis(alkylcarbonyl)imido groups, bis(alkylsulfonyl)methylene groups, bis(alkylsulfonyl)imido groups, tris(alkylcarbonyl)methylene groups, and tris(alkylsulfonyl)methylene groups; and alcoholic hydroxyl groups.

In particular, the polar group is preferably a carboxy group, a phenolic hydroxy group, a fluorinated alcohol group (preferably a hexafluoroisopropanol group), or a sulfonic acid group.

Examples of the leaving group that leaves by the action of an acid include groups represented by formulas (Y1) to (Y4).

—C(Rx₁)(Rx₂)(Rx₃) Formula (Y1):

—C(═O)OC(Rx₁)(Rx₂)(Rx₃) Formula (Y2):

—C(R₃₆)(R₃₇)(OR₃₈) Formula (Y3):

—C(Rn)(H)(Ar) Formula (Y4):

In formulas (Y1) and (Y2), Rx₁ to Rx₃ each independently represent an alkyl group (linear or branched alkyl group), a cycloalkyl group (monocyclic or polycyclic cycloalkyl group), an alkenyl group (linear or branched alkenyl group), or an aryl group (monocyclic or polycyclic aryl group). When all of Rx₁ to Rx₃ are alkyl groups (linear or branched alkyl groups), it is preferable that at least two selected from the group consisting of Rx₁ to Rx₃ are each a methyl group.

In particular, it is preferable that Rx₁ to Rx₃ each independently represent a linear or branched alkyl group, and it is more preferable that Rx₁ to Rx₃ each independently represent a linear alkyl group.

Two selected from the group consisting of Rx₁ to Rx₃ may be bonded together to form a monocyclic or polycyclic ring.

The alkyl group represented by each of Rx₁ to Rx₃ is preferably an alkyl group having 1 to 5 carbon atoms such as a methyl group, an ethyl group, a n-propyl group, an isopropyl group, a n-butyl group, an isobutyl group, or a t-butyl group.

The cycloalkyl group represented by each of Rx₁ to Rx₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group.

The aryl group represented by each of Rx₁ to Rx₃ is preferably an aryl group having 6 to 10 carbon atoms, and examples thereof include a phenyl group, a naphthyl group, and an anthryl group.

The alkenyl group represented by each of Rx₁ to Rx₃ is preferably a vinyl group.

The ring formed by bonding two selected from the group consisting of Rx₁ to Rx₃ is preferably a cycloalkyl group. The cycloalkyl group formed by bonding two selected from the group consisting of Rx₁ to Rx₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group and is more preferably a monocyclic cycloalkyl group having 5 to 6 carbon atoms.

One methylene group included in the ring in the cycloalkyl group formed by bonding two selected from the group consisting of Rx₁ to Rx₃ may be replaced with a heteroatom such as an oxygen atom, a group including a heteroatom such as a carbonyl group, or a vinylidene group. In any of these cycloalkyl groups, at least one ethylene group included in the cycloalkane ring may be replaced with a vinylene group.

In the group represented by formula (Y1) or formula (Y2), it is preferable that, for example, Rx₁ is a methyl group or an ethyl group and that Rx₂ and Rx₃ are bonded together to form the cycloalkyl group described above.

When the resist composition is, for example, a resist composition for EUV exposure, it is also preferable that the alkyl, cycloalkyl, alkenyl, and aryl groups represented by Rx₁ to Rx₃ and the ring formed by bonding two selected from the group consisting of Rx₁ to Rx₃ each further have a fluorine atom or an iodine atom as a substituent.

In formula (Y3), R₃₆ to R₃₈ each independently represent a hydrogen atom or a monovalent organic group. R³⁷ and R³⁸ may be bonded together to form a ring. Examples of the monovalent organic group include alkyl groups, cycloalkyl groups, aryl groups, aralkyl groups, and alkenyl groups. It is also preferable that R³⁶ is a hydrogen atom.

The alkyl, cycloalkyl, aryl, and aralkyl groups described above may each include a heteroatom such as an oxygen atom and/or a group including a heteroatom such as a carbonyl group. For example, in the alkyl, cycloalkyl, aryl, and aralkyl groups described above, at least one methylene group may be replaced with a heteroatom such as an oxygen atom and/or a group including a heteroatom such as a carbonyl group.

R₃₈ may be bonded to another substituent included in the main chain of the repeating unit to form a ring. The group formed by bonding R₃₈ and another substituent included in the main chain of the repeating unit is preferably an alkylene group such as a methylene group.

When the resist composition is, for example, a resist composition for EUV exposure, it is also preferable that the monovalent organic groups represented by R₃₆ to R₃₈ and the group formed by bonding R₃₇ and R₃₈ together each further have a fluorine atom or an iodine atom as a substituent.

Formula (Y3) is preferably a group represented by the following formula (Y3-1).

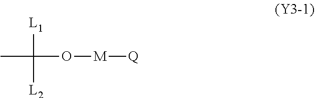

L₁ and L₂ each independently represent a hydrogen atom, an alkyl group, a cycloalkyl group, an aryl group, or a group formed by combining any of them (for example, a group formed by combining an alkyl group and an aryl group).

M represents a single bond or a divalent linking group.

Q represents an alkyl group optionally including a heteroatom, a cycloalkyl group optionally including a heteroatom, an aryl group optionally including a heteroatom, an amino group, an ammonium group, a mercapto group, a cyano group, an aldehyde group, or a group formed by combining any of them (for example, a group formed by combining an alkyl group and a cycloalkyl group).

In the alkyl and cycloalkyl groups, for example, one methylene group may be replaced with a heteroatom such as an oxygen atom or a group including a heteroatom such as a carbonyl group.

It is preferable that one of L₁ or L₂ is a hydrogen atom and that the other is an alkyl group, a cycloalkyl group, an aryl group, or a group formed by combining an alkylene group and an aryl group.

At least two selected from the group consisting of Q, M, and L₁ may be bonded together to form a ring (preferably a 5-membered or 6-membered ring).

From the viewpoint of obtaining a finer pattern, L₂ is preferably a secondary or tertiary alkyl group and more preferably a tertiary alkyl group. Examples of the secondary alkyl group include an isopropyl group, a cyclohexyl group, and a norbornyl group, and examples of the tertiary alkyl group include a tert-butyl group and an adamantane group. In these modes, since Tg (glass transition temperature) and activation energy are high, high film hardness is obtained, and the occurrence of fogging can be reduced.

When the composition of the invention is, for example, an actinic ray-sensitive or radiation-sensitive resin composition for EUV exposure, it is also preferable that the alkyl, cycloalkyl, and aryl groups represented by L₁ and L₂ and a group formed by combining any of these groups each further have a fluorine atom or an iodine atom as a substituent. It is also preferable that the alkyl, cycloalkyl, aryl, and aralkyl groups each include a heteroatom such as an oxygen atom other than a fluorine atom and an iodine atom (i.e., in the alkyl, cycloalkyl, aryl, and aralkyl groups, for example, one methylene group is replaced with a heteroatom such as an oxygen atom or a group including a heteroatom such as a carbonyl group).

When the composition of the invention is, for example, a resist composition for EUV exposure, it is also preferable that, in the alkyl group optionally including a heteroatom, the cycloalkyl group optionally including a heteroatom, the aryl group optionally including a heteroatom, the amino group, the ammonium group, the mercapto group, the cyano group, and the aldehyde group that are represented by Q and a combination of any of these groups, the heteroatom is one selected from the group consisting of a fluorine atom, an iodine atom, and an oxygen atom.

In formula (Y4), Ar represents an aromatic ring group. Rn represents an alkyl group, a cycloalkyl group, or an aryl group. Rn and Ar may be bonded together to form a non-aromatic ring. Ar is preferably an aryl group.

When the composition of the invention is, for example, a resist composition for EUV exposure, it is also preferable that the aromatic ring group represented by Ar and the alkyl, cycloalkyl, or aryl group represented by Rn each have a fluorine atom or an iodine atom as a substituent.

When, in the leaving group protecting the polar group, a non-aromatic ring is bonded directly to the polar group (or its residue), it is also preferable that a ring member atom adjacent to the ring member atom bonded directly to the polar group (or its residue) in the non-aromatic ring does not have a halogen atom such as a fluorine atom as a substituent, because the repeating unit can have good acid-decomposability.

The leaving group that leaves by the action of an acid may also be a 2-cyclopentenyl group having a substituent (e.g., an alkyl group) such as a 3-methyl-2-cyclopentenyl group or a cyclohexyl group having a substituent (e.g., an alkyl group) such as a 1,1,4,4-tetramethylcyclohexyl group.

The repeating unit having an acid-decomposable group is also preferably a repeating unit represented by formula (A).

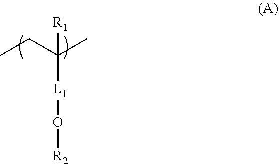

L₁ represents a divalent linking group optionally having a fluorine atom or an iodine atom, and R₁ represents a hydrogen atom, a fluorine atom, an iodine atom, an alkyl group optionally having a fluorine atom or an iodine atom, or an aryl group optionally having a fluorine atom or an iodine atom. R₂ represents a leaving group that optionally has a fluorine atom or an iodine atom and leaves by the action of an acid. At least one of L₁, R₁, or R₂ has a fluorine atom or an iodine atom.

L₁ represents a divalent linking group optionally having a fluorine atom or an iodine atom. Examples of the divalent linking group optionally having a fluorine atom or an iodine atom include —CO—, —O—, —S—, —SO—, —SO₂—, hydrocarbon groups optionally having a fluorine atom or an iodine atom (such as alkylene groups, cycloalkylene groups, alkenylene groups, and arylene groups), and linking groups formed by linking a plurality of groups selected from the above groups. In particular, L₁ is preferably —CO—, an arylene group, or -arylene group-fluorine or iodine atom-containing alkylene group- and more preferably —CO— or -arylene group-fluorine or iodine atom-containing alkylene group-.

The arylene group is preferably a phenylene group.

The alkylene group may by a linear or branched alkylene group. No particular limitation is imposed on the number of carbon atoms in the alkylene group, but the number of carbon atoms is preferably 1 to 10 and more preferably 1 to 3.

No particular limitation is imposed on the total number of fluorine or iodine atoms included in the fluorine or iodine atom-containing alkylene group, but the total number of fluorine or iodine atoms is preferably two or more, more preferably 2 to 10, and still more preferably 3 to 6.

R₁ represents a hydrogen atom, a fluorine atom, an iodine atom, an alkyl group optionally having a fluorine atom or an iodine atom, or an aryl group optionally having a fluorine atom or an iodine atom.

The alkyl group may be a linear or branched alkyl group. No particular limitation is imposed on the number of carbon atoms in the alkyl group, but the number of carbon atoms is preferably 1 to 10 and more preferably 1 to 3.

No particular limitation is imposed on the total number of fluorine or iodine atoms included in the fluorine or iodine atom-containing alkyl group, but the total number of fluorine or iodine atoms is preferably 1 or more, more preferably 1 to 5, and still more preferably 1 to 3.

The alkyl group may include a heteroatom such as an oxygen atom other than halogen atoms.

R² represents a leaving group that leaves by the action of an acid and that optionally has a fluorine atom or an iodine atom. Examples of the leaving group optionally having a fluorine atom or an iodine atom include leaving groups represented by formulas (Y1) to (Y4) described above and having a fluorine atom or an iodine atom.

It is also preferable that the repeating unit having an acid-decomposable group is a repeating unit represented by formula (AI).

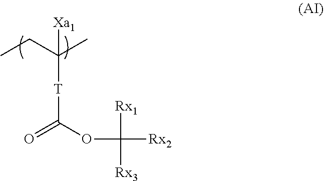

In formula (AI), Xa₁ represents a hydrogen atom or an alkyl group optionally having a substituent. T represents a single bond or a divalent linking group. Rx₁ to Rx₃ each independently represent an alkyl group (linear or branched alkyl group), a cycloalkyl group (monocyclic or polycyclic cycloalkyl group), an alkenyl group (linear or branched alkenyl group), or an aryl group (monocyclic or polycyclic aryl group). However, when all of Rx₁ to Rx₃ are alkyl groups (linear or branched alkyl groups), it is preferable that at least two selected from the group consisting of Rx₁ to Rx₃ are each a methyl group.

Two selected from the group consisting of Rx₁ to Rx₃ may be bonded together to form a monocyclic or polycyclic group (such as a monocyclic or polycyclic cycloalkyl group).

Examples of the alkyl group optionally having a substituent and represented by Xa₁ include a methyl group and a group represented by —CH₂—R₁₁. R₁₁ represents a halogen atom (such as a fluorine atom), a hydroxy group, or a monovalent organic group, and examples thereof include alkyl groups having 5 or less carbon atoms and optionally substituted with a halogen atom, acyl groups having 5 or less carbon atoms and optionally substituted with a halogen atom, and alkoxy groups having 5 or less carbon atoms and optionally substituted with a halogen atom. R₁₁ is preferably an alkyl group having 3 or less carbon atoms and more preferably a methyl group. Xa₁ is preferably a hydrogen atom, a methyl group, a trifluoromethyl group, or a hydroxymethyl group.

Examples of the divalent linking group represented by T include alkylene groups, aromatic ring groups, a —COO-Rt-group, and an —O-Rt-group. In these formulas, Rt represents an alkylene group or a cycloalkylene group.

T is preferably a single bond or a —COO-Rt-group. When T represents a —COO-Rt-group, Rt is preferably an alkylene group having 1 to 5 carbon atoms and more preferably a —CH₂-group, a —(CH₂)₂— group, or a —(CH₂)₃— group.

The alkyl group represented by each of Rx₁ to Rx₃ is preferably an alkyl group having 1 to 4 carbon atoms such as a methyl group, an ethyl group, a n-propyl group, an isopropyl group, a n-butyl group, an isobutyl group, or a t-butyl group.

The cycloalkyl group represented by each of Rx₁ to Rx₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group.

The aryl group represented by each of Rx₁ to Rx₃ is preferably an aryl group having 6 to 10 carbon atoms, and examples thereof include a phenyl group, a naphthyl group, and an anthryl group.

The alkenyl group represented by each of Rx₁ to Rx₃ is preferably a vinyl group.

The cycloalkyl group formed by bonding two selected from the group consisting of Rx₁ to Rx₃ is preferably a monocyclic cycloalkyl group such as a cyclopentyl group or a cyclohexyl group. The cycloalkyl group is also preferably a polycyclic cycloalkyl group such as a norbornyl group, a tetracyclodecanyl group, a tetracyclododecanyl group, or an adamantyl group. In particular, a monocyclic cycloalkyl group having 5 to 6 carbon atoms is preferred.

In the cycloalkyl group formed by bonding two selected from the group consisting of Rx₁ to Rx₃, for example, one methylene group included in the ring may be replaced with a heteroatom such as an oxygen atom, a group including a heteroatom such as a carbonyl group, or a vinylidene group. In each cycloalkyl group, at least one ethylene group included in the cycloalkane ring may be replaced with a vinylene group.

In the repeating unit represented by formula (AI), it is preferable that, for example, Rx₁ is a methyl group or an ethyl group and that Rx₂ and Rx₃ are bonded together to form the cycloalkyl group described above.

When any of the above-described groups has a substituent, examples of the substituent include alkyl groups (having 1 to 4 carbon atoms), halogen atoms, a hydroxy group, alkoxy groups (having 1 to 4 carbon atoms), a carboxy group, and alkoxycarbonyl groups (having 2 to 6 carbon atoms). The number of carbon atoms in the substituent is preferably 8 or less.

The repeating unit represented by formula (AI) is preferably an acid-decomposable tertiary alkyl (meth)acrylate-based repeating unit (a repeating unit in which Xa₁ represents a hydrogen atom or a methyl group and T represents a single bond).

Specific examples of the repeating unit having an acid-decomposable group are shown below, but the present invention is not limited thereto. In the formulas below, Xa₁ represents H, CH₃, CF₃, or CH₂OH, and Rxa and Rxb each independently represent a linear or branched alkyl group having 1 to 5 carbon atoms.

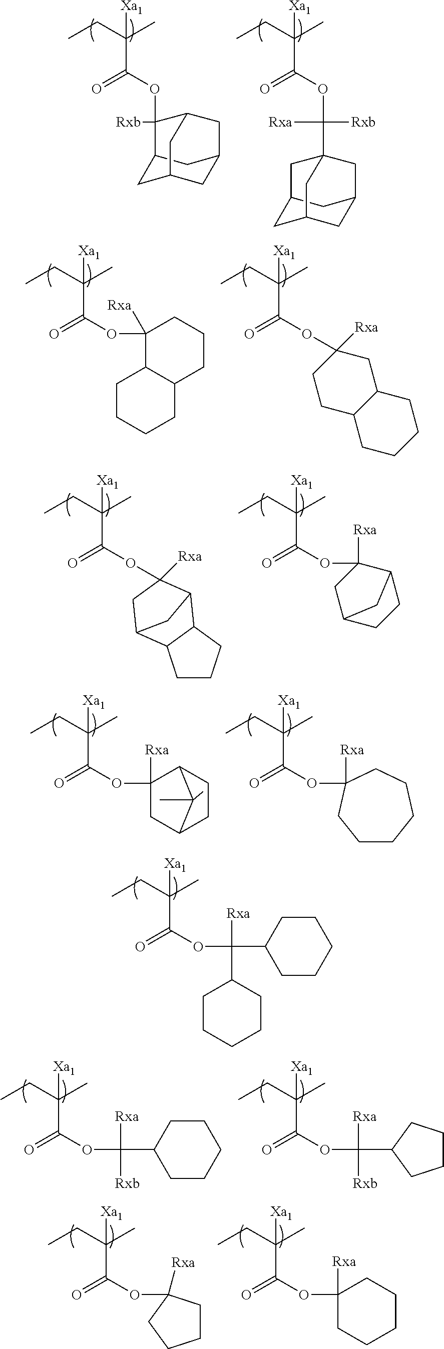

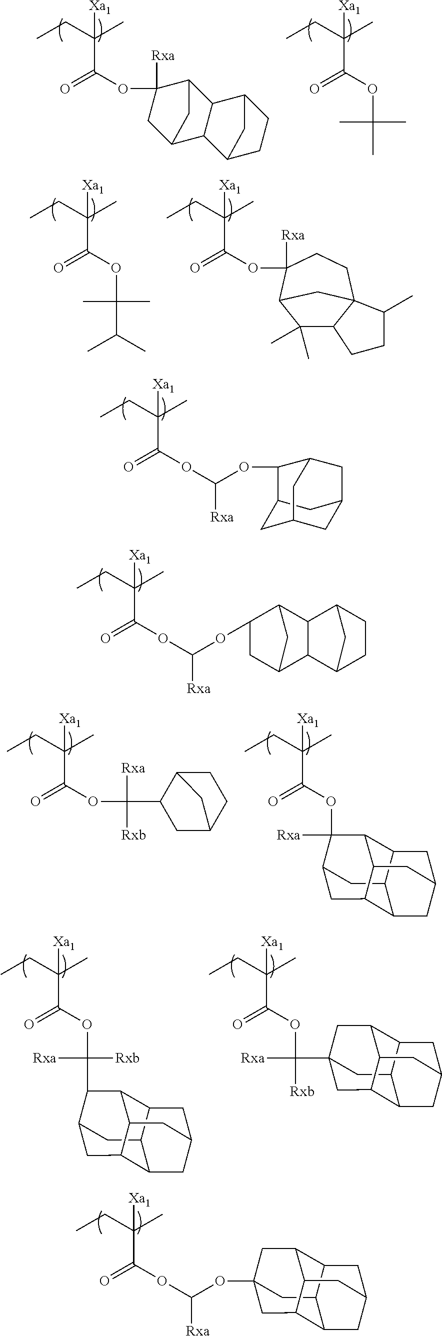

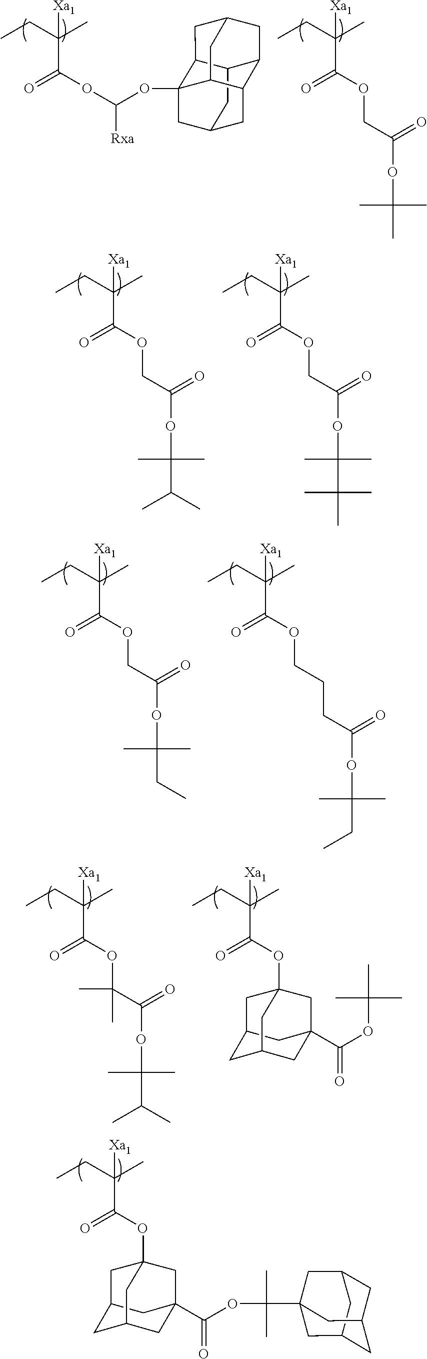

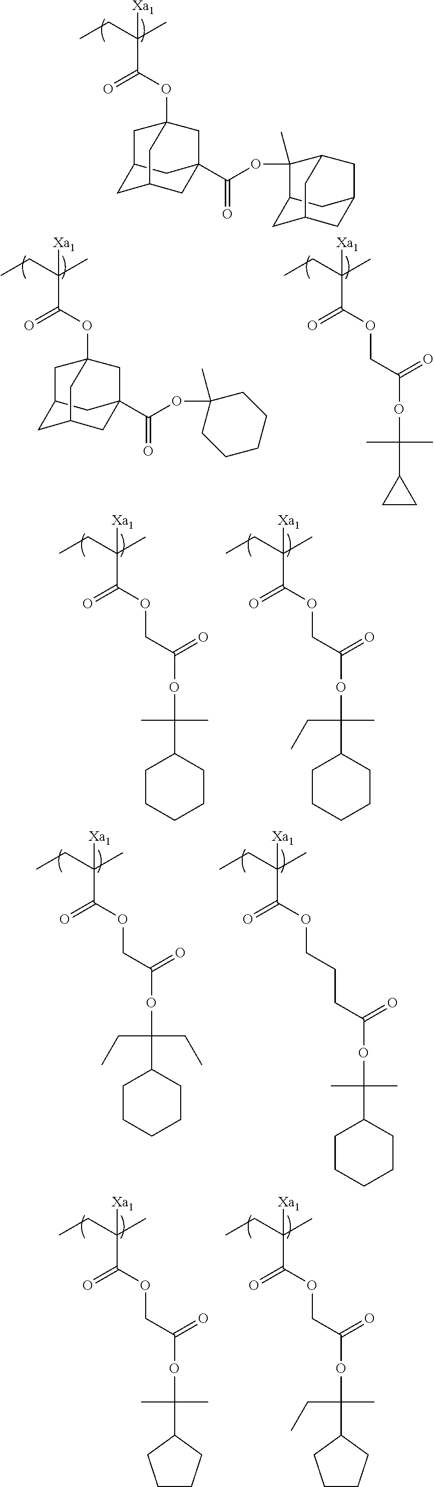

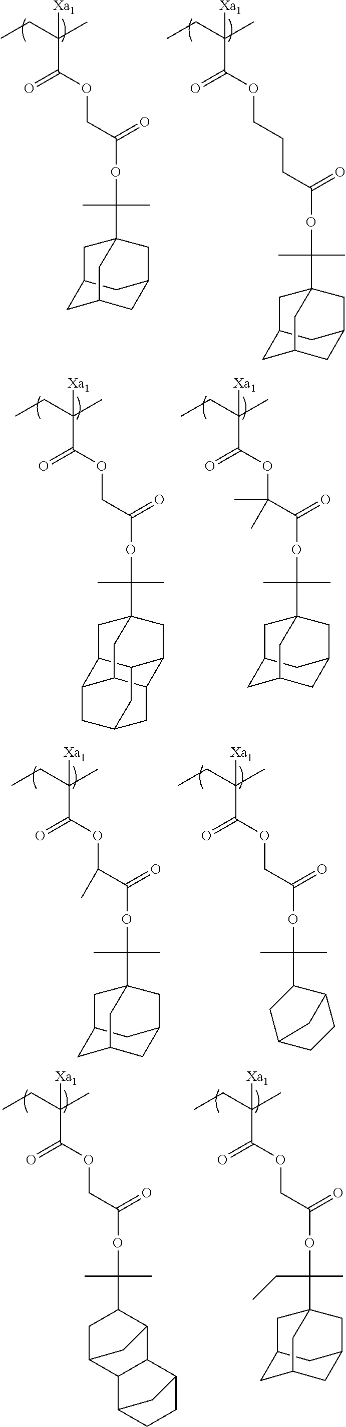

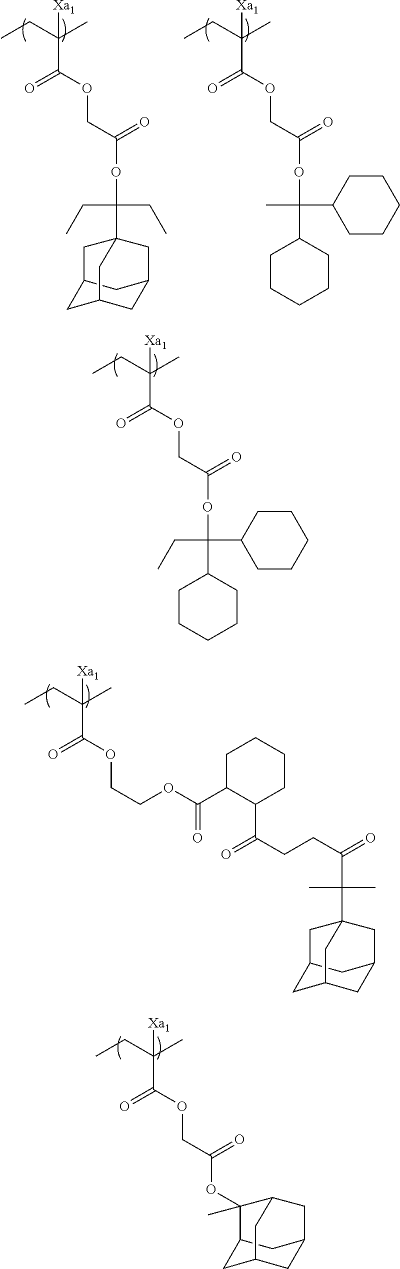

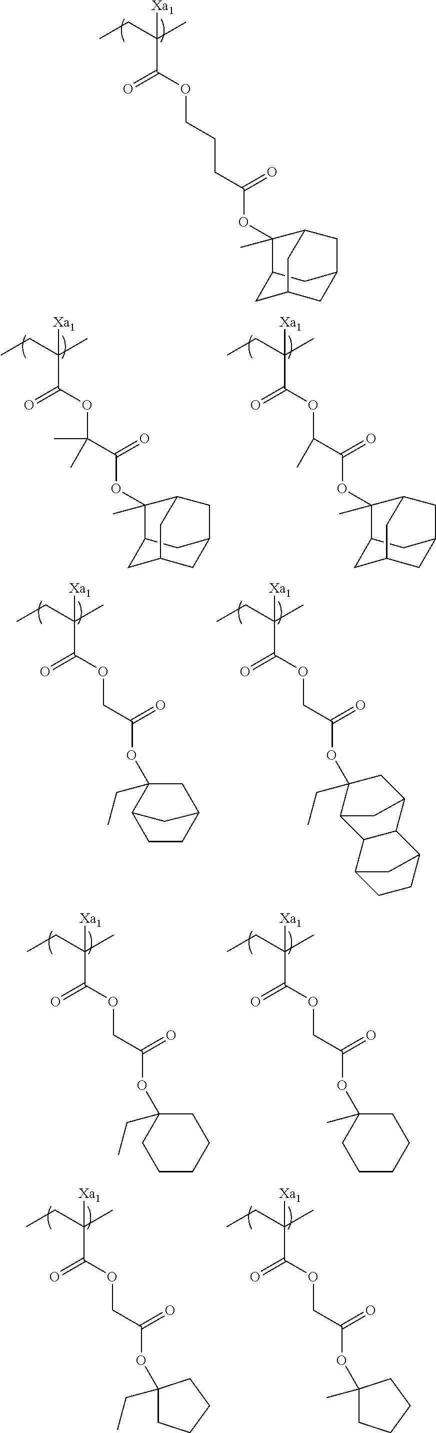

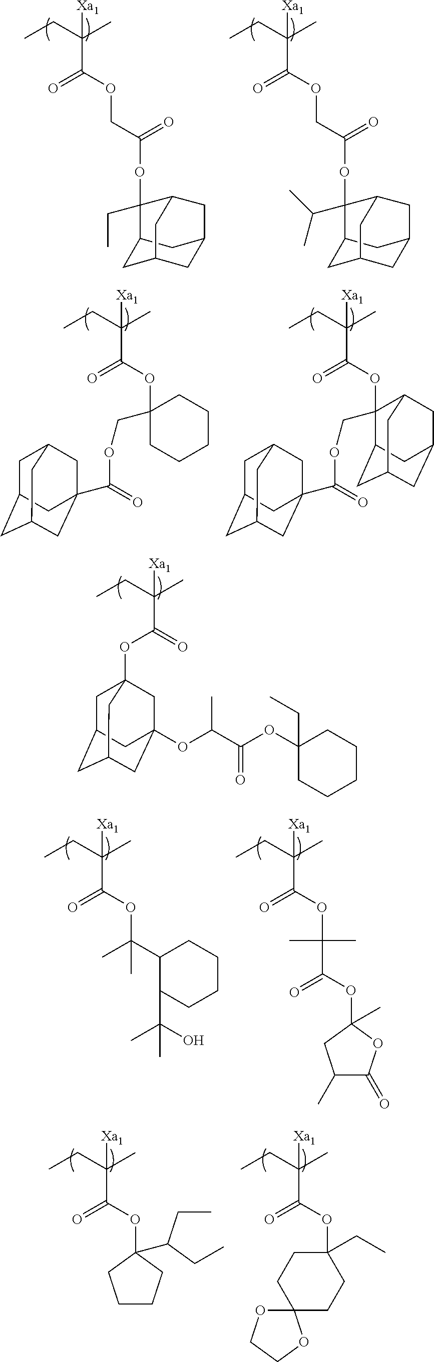

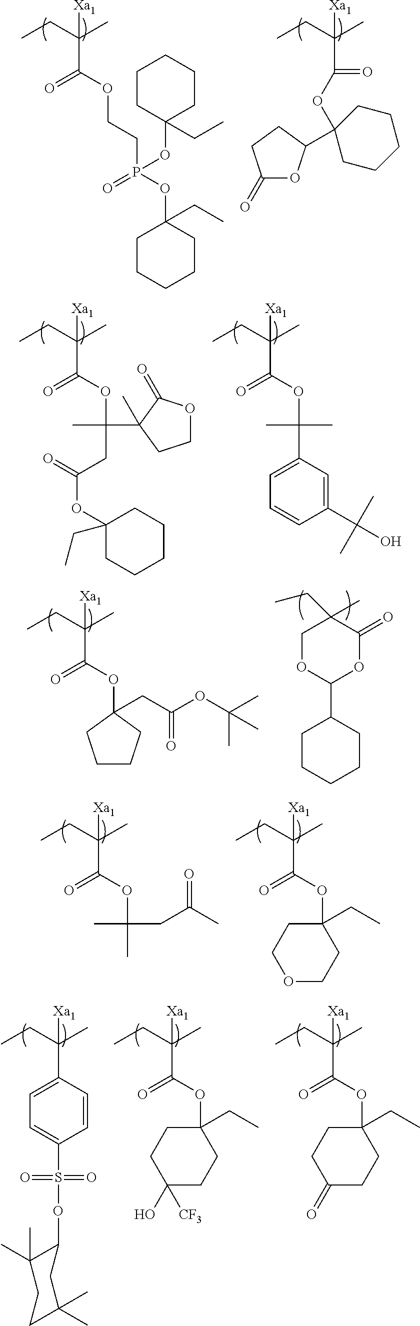

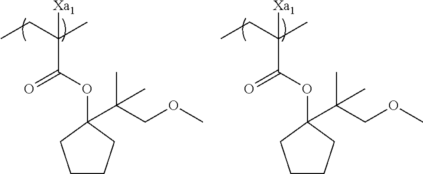

The resin (A) may include, as the repeating unit having the acid-decomposable group, a repeating unit having an acid-decomposable group including an unsaturated bond.

The repeating unit having an acid-decomposable group including an unsaturated bond is preferably a repeating unit represented by formula (B).

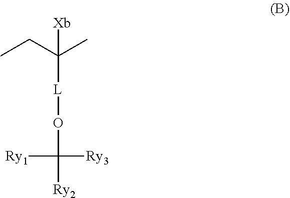

In formula (B), Xb represents a hydrogen atom, a halogen atom, or an alkyl group optionally having a substituent. L represents a single bond or a divalent linking group optionally having a substituent. Ry₁ to Ry₃ each independently represent a linear or branched alkyl group, a monocyclic or polycyclic cycloalkyl group, an alkenyl group, an alkynyl group, or a monocyclic or polycyclic aryl group. However, at least one of Ry₁, Ry₂, or Ry₃ represents an alkenyl group, an alkynyl group, a monocyclic or polycyclic cycloalkenyl group, or a monocyclic or polycyclic aryl group.

Two selected from the group consisting of Ry₁ to Ry₃ may be bonded together to form a monocyclic or polycyclic ring (such as a monocyclic or polycyclic cycloalkyl group or a monocyclic or polycyclic cycloalkenyl group).

The alkyl group optionally having a substituent and represented by Xb is, for example, a methyl group or a group represented by —CH₂-R¹¹. R₁₁ represents a halogen atom (such as a fluorine atom), a hydroxy group, or a monovalent organic group, and examples thereof include alkyl groups having 5 or less carbon atoms and optionally substituted with a halogen atom, acyl groups having 5 or less carbon atoms and optionally substituted with a halogen atom, and alkoxy groups having 5 or less carbon atoms and optionally substituted with a halogen atom. Rn is preferably an alkyl group having 3 or less carbon atoms and more preferably a methyl group. Xb is preferably a hydrogen atom, a fluorine atom, a methyl group, a trifluoromethyl group, or a hydroxymethyl group.

Examples of the divalent linking group represented by L include an -Rt- group, a —CO— group, a —COO-Rt- group, a —COO-Rt-CO— group, an -Rt-CO— group, and an —O-Rt- group. In these formulas, Rt represents an alkylene group, a cycloalkylene group, or an aromatic ring group and is preferably an aromatic ring group.

L is preferably an -Rt- group, a —CO— group, a —COO-Rt-CO— group, or an -Rt-CO— group. Rt may have a substituent such as a halogen atom, a hydroxy group, or an alkoxy group. Rt is preferably an aromatic group.

The alkyl group represented by each of Ry₁ to Ry₃ is preferably an alkyl group having 1 to 4 carbon atoms such as a methyl group, an ethyl group, a n-propyl group, an isopropyl group, a n-butyl group, an isobutyl group, or a t-butyl group.

The cycloalkyl group represented by each of Ry₁ to Ry₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group.

The aryl group represented by each of Ry₁ to Ry₃ is preferably an aryl group having 6 to 10 carbon atoms, and examples thereof include a phenyl group, a naphthyl group, and an anthryl group.

The alkenyl group represented by each of Ry₁ to Ry₃ is preferably a vinyl group.

The alkynyl group represented by each of Ry₁ to Ry₃ is preferably an ethynyl group.

The cycloalkenyl group represented by each of Ry₁ to Ry₃ is preferably a structure including a monocyclic cycloalkyl group such as a cyclopentyl group or a cyclohexyl group with a double bond present in part of the monocyclic cycloalkyl group.

The cycloalkyl group formed by bonding two selected from the group consisting of Ry₁ to Ry₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group. In particular, the cycloalkyl group is more preferably a monocyclic cycloalkyl group having 5 to 6 carbon atoms.

In the cycloalkyl or cycloalkenyl group formed by bonding two selected from the group consisting of Ry₁ to Ry₃, for example, one methylene group included in the ring may be replaced with a heteroatom such as an oxygen atom, a group including a heteroatom such as a carbonyl group, an —SO₂— group, or an —SO₃— group, a vinylidene group, or a combination thereof. In the cycloalkyl or cycloalkenyl group, at least one ethylene group included in the cycloalkane or cycloalkenyl ring may be replaced with a vinylene group.

In the repeating unit represented by formula (B), it is preferable that, for example, Ry₁ is a methyl group, an ethyl group, a vinyl group, an allyl group, or an aryl group and that Rye and Ry₃ are bonded together to form the cycloalkyl or cycloalkenyl group described above.

When any of the groups described above has a substituent, examples of the substituent include alkyl groups (having 1 to 4 carbon atoms), halogen atoms, a hydroxy group, alkoxy groups (having 1 to 4 carbon atoms), a carboxy group, and alkoxycarbonyl groups (having 2 to 6 carbon atoms). The number of carbon atoms in the substituent is preferably 8 or less.

The repeating unit represented by formula (B) is preferably an acid-decomposable (meth)acrylic acid tertiary ester-based repeating unit (a repeating unit in which Xb represents a hydrogen atom or a methyl group and L represents a —CO— group), an acid-decomposable hydroxystyrene tertiary alkyl ether-based repeating unit (a repeating unit in which Xb represents a hydrogen atom or a methyl group and L represents a phenyl group), or an acid-decomposable styrenecarboxylic acid tertiary ester-based repeating unit (a repeating unit in which Xb represents a hydrogen atom or a methyl group and L represents an -Rt-CO— group (Rt is an aromatic group)).

The content of the repeating unit having the acid-decomposable group including an unsaturated bond with respect to the total amount of the repeating units in the resin (A) is preferably 15% by mole or more, more preferably 20% by mole or more, and still more preferably 30% by mole or more. The upper limit of the content of the repeating unit with respect to the total amount of the repeating units in the resin (A) is preferably 80% by mole or less, more preferably 70% by mole or less, and particularly preferably 60% by mole or less.

Specific example of the repeating unit having the acid-decomposable group including an unsaturated bond are shown below, but the invention is not limited thereto. In the formulas below, Xb and L₁ each represent any of the above-described substituents and linking groups, and Ar represents an aromatic group. R represents a substituent such as a hydrogen atom, an alkyl group, a cycloalkyl group, an aryl group, an aralkyl group, an alkenyl group, a hydroxy group, an alkoxy group, an acyloxy group, a cyano group, a nitro group, an amino group, a halogen atom, an ester group (—OCOR′″ or —COOR′″: R′″ represents an alkyl group having 1 to 20 carbon atoms or a fluorinated alkyl group having 1 to 20 carbon atoms), or a carboxy group, and R′ represents a linear or branched alkyl group, a monocyclic or polycyclic cycloalkyl group, an alkenyl group, an alkynyl group, or a monocyclic or polycyclic aryl group. Q represents a heteroatom such as an oxygen atom, a group including a heteroatom such as a carbonyl group, an —SO₂— group, or an —SO₃— group, a vinylidene group, or a combination thereof. l, n and m each represent an integer of 0 or more.

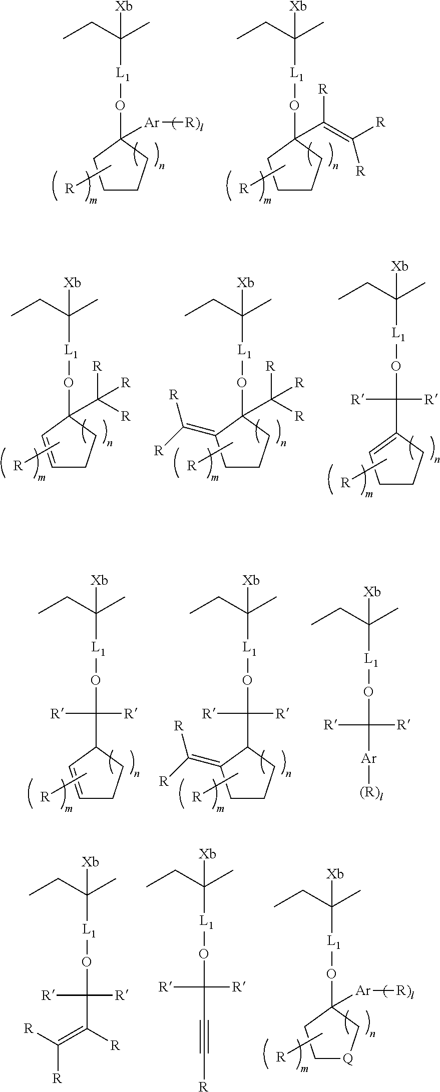

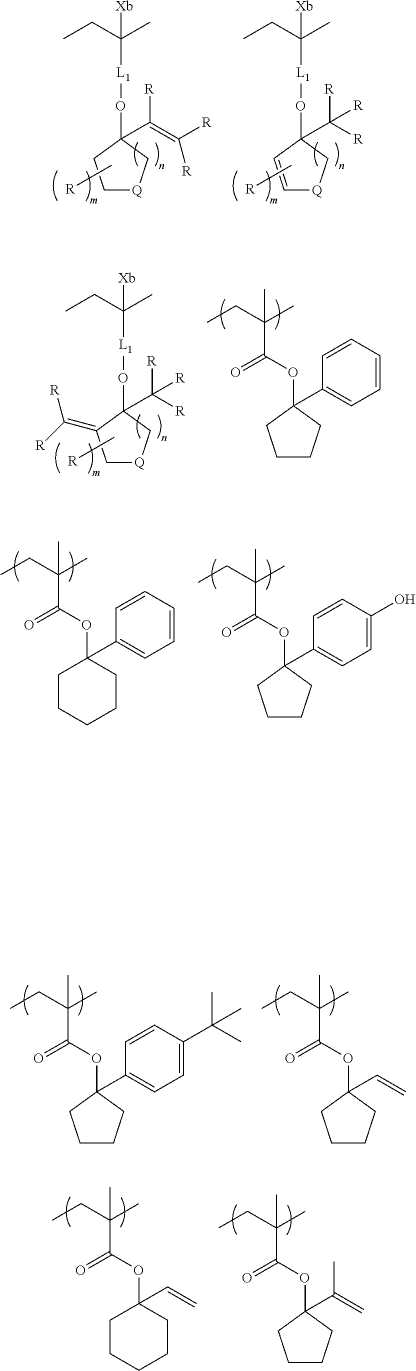

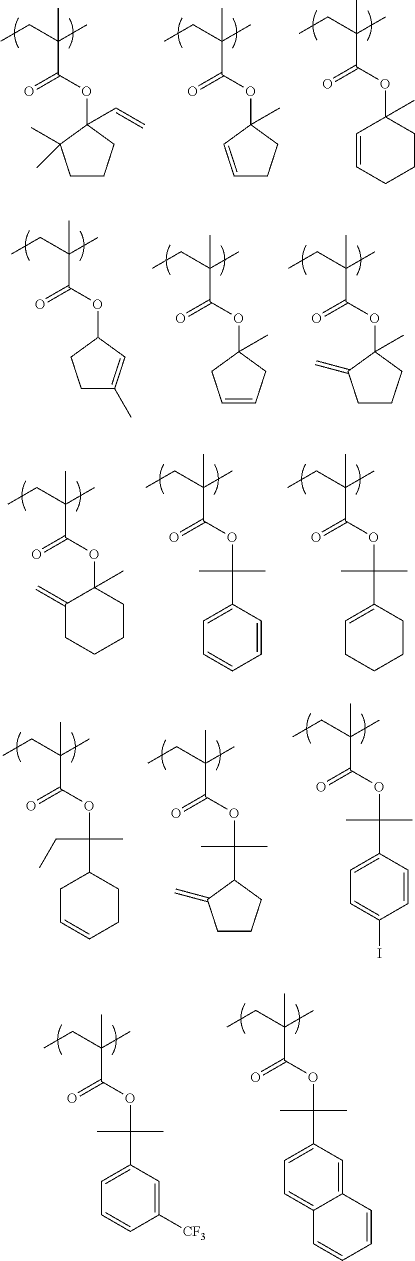

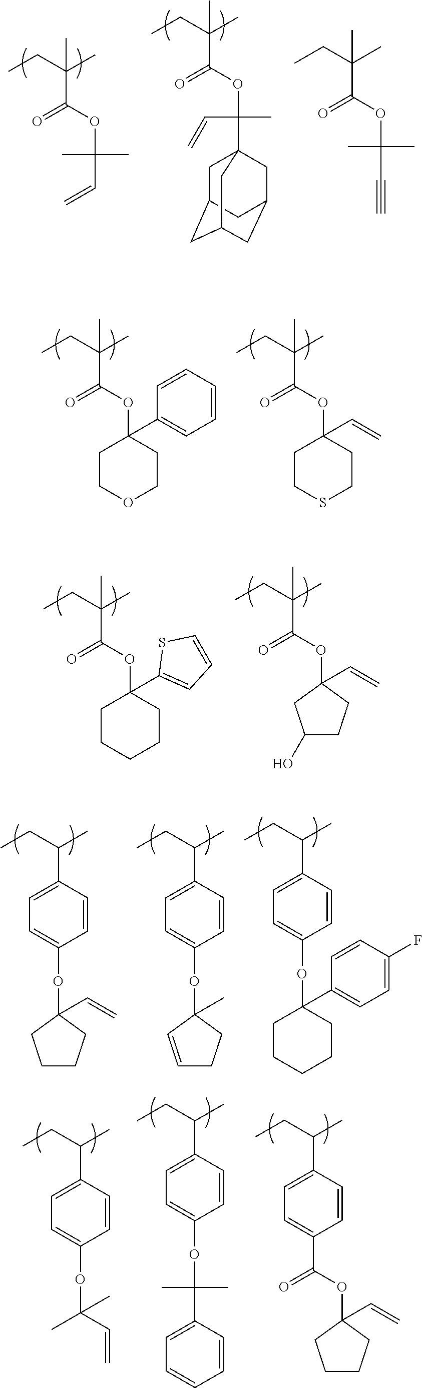

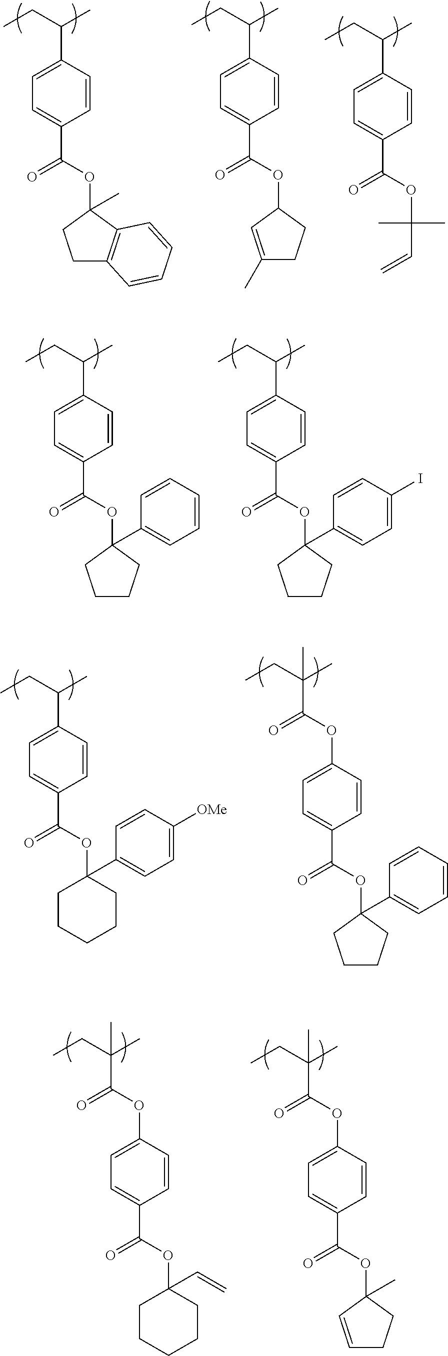

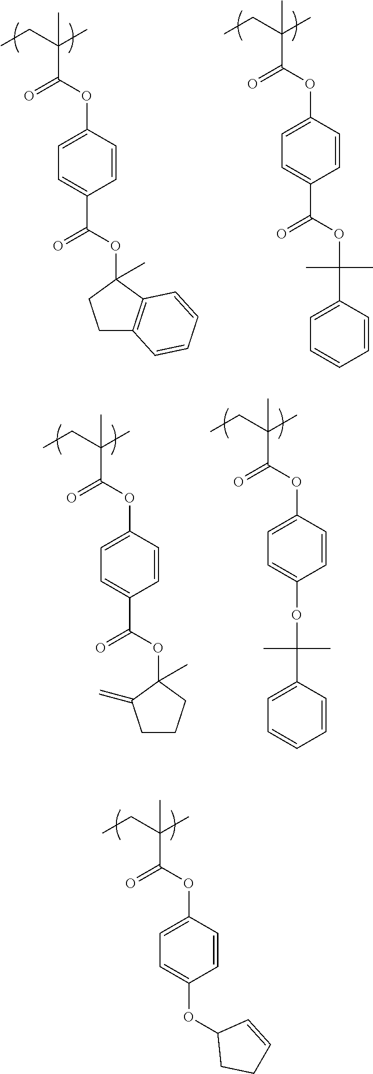

The resin (A) may include only one type of repeating unit having the acid-decomposable group or may include two or more types in combination.

The content of the repeating unit having the acid-decomposable group with respect to the total amount of the repeating units in the resin (A) is preferably 15% by mole or more, more preferably 20% by mole or more, and still more preferably 30% by mole or more. The upper limit of the content of the repeating unit with respect to the total amount of the repeating units in the resin (A) is preferably 90% by mole or less, more preferably 80% by mole or less, still more preferably 70% by mole or less, and particularly preferably 60% by mole or less.

The resin (A) may include at least one repeating unit selected from the following group A and/or at least one repeating unit selected from the following group B.

Group A: The group consisting of the following repeating units (20) to (29).

(20) A repeating unit having an acid group, which will be described later.

(21) A repeating unit having no acid-decomposable group and no acid group but having a fluorine atom, a bromine atom, or an iodine atom, which will be described later.

(22) A repeating unit having a lactone group, a sultone group, or a carbonate group, which will be described later.

(23) A repeating unit having a photoacid generating group, which will be described later.

(24) A repeating unit represented by formula (V-1) or formula (V-2) below, which will be described later.

(25) A repeating unit represented by formula (A), which will be described later.

(26) A repeating unit represented by formula (B), which will be described later.

(27) A repeating unit represented by formula (C), which will be described later.

(28) A repeating unit represented by formula (D), which will be described later.

(29) A repeating unit represented by formula (E), which will be described later.

Group B: The group consisting of the following repeating units (30) to (32).

(30) A repeating unit having at least one group selected from the group consisting of lactone groups, sultone groups, carbonate groups, a hydroxy group, a cyano group, and alkali-soluble groups, which will be described later.

(31) A repeating unit having an alicyclic hydrocarbon structure and exhibiting no acid decomposability, which will be described later.

(32) A repeating unit represented by formula (III) and having no hydroxy group and no cyano group, which will be described later.

The resin (A) has preferably an acid group and includes preferably a repeating unit having an acid group as described later. The definition of the acid group will be described later along with preferred modes of the repeating unit having an acid group. When the resin (A) has the acid group, the interaction between the resin (A) and the acid generated from the photoacid generator is enhanced. This results in a further reduction in diffusion of the acid, and a pattern to be formed may have a sharper rectangular cross-sectional shape.

When the composition of the invention is used as an actinic ray-sensitive or radiation-sensitive resin composition for EUV light, it is preferable that the resin (A) has at least one repeating unit selected from the group A.

When the composition of the invention is used as an actinic ray-sensitive or radiation-sensitive resin composition for EUV light, it is preferable that the resin (A) includes at least one of a fluorine atom or an iodine atom. When the resin (A) includes both a fluorine atom and an iodine atom, the resin (A) may have one type of repeating unit including both a fluorine atom and an iodine atom or may include two types of repeating units including a repeating unit including a fluorine atom and a repeating unit including an iodine atom.

When the composition of the invention is used as an actinic ray-sensitive or radiation-sensitive resin composition for EUV light, it is also preferable that the resin (A) has a repeating unit having an aromatic group.

When the composition of the invention is used as an actinic ray-sensitive or radiation-sensitive resin composition for ArF light, it is preferable that the resin (A) has at least one type of repeating unit selected from the group B.

When the composition of the invention is used as an actinic ray-sensitive or radiation-sensitive resin composition for ArF light, it is preferable that the resin (A) includes no fluorine atom and no silicon atom.

When the composition of the invention is used as an actinic ray-sensitive or radiation-sensitive resin composition for ArF light, it is preferable that the resin (A) has no aromatic group.

### (Repeating Unit Having Acid Group)

The resin (A) may have a repeating unit having an acid group.

The acid group is preferably an acid group having a pKa of 13 or less. The acid dissociation constant of the acid group is preferably 13 or less, more preferably 3 to 13, and still more preferably 5 to 10.

When the resin (A) has the acid group having a pKa of 13 or less, no particular limitation is imposed on the content of the acid group in the resin (A), but the content is often 0.2 to 6.0 mmol/g. In particular, the content is preferably 0.8 to 6.0 mmol/g, more preferably 1.2 to 5.0 mmol/g, and still more preferably 1.6 to 4.0 mmol/g. When the content of the acid group is within the above range, development proceeds smoothly, and a pattern to be formed has a good profile, so that high resolution is achieved.

The acid group is preferably, for example, a carboxy group, a phenolic hydroxy group, a fluorinated alcohol group (preferably a hexafluoroisopropanol group), a sulfonic group, a sulfonamido group, or an isopropanol group.

In the hexafluoroisopropanol group, one or more (preferably one to two) fluorine atoms may each be replaced with a group other than a fluorine atom (such as an alkoxycarbonyl group). The acid group is also preferably —C(CF₃)(OH)—CF₂— formed as described above. At least one fluorine atom may be replaced with a group other than a fluorine atom to form a ring including —C(CF₃)(OH)—CF₂—.

Preferably, the repeating unit having the acid group is a repeating unit different from the above-described repeating unit having a structure in which a polar group is protected by a leaving group that leaves by the action of an acid and from a repeating unit having a lactone group, a sultone group, or a carbonate group that is described later.

The repeating unit having the acid group may have a fluorine atom or an iodine atom.

Examples of the repeating unit having the acid group include the following repeating units.

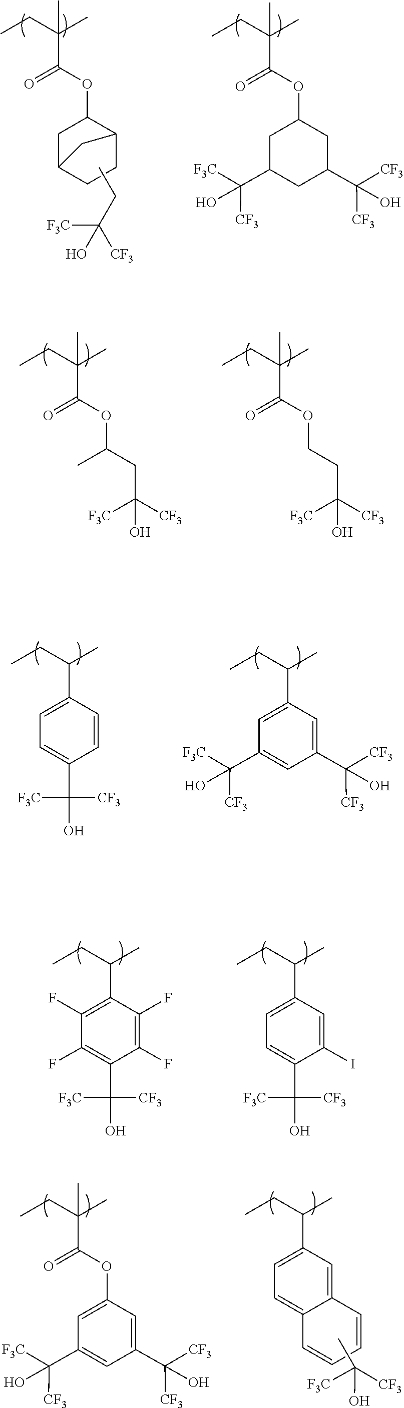

The repeating unit having the acid group is preferably a repeating unit represented by the following formula (1).

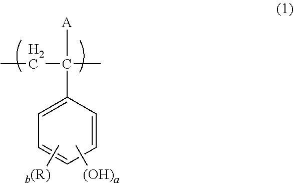

In formula (1), A represents a hydrogen atom, an alkyl group, a cycloalkyl group, a halogen atom, or a cyano group. R represents a halogen atom, an alkyl group, a cycloalkyl group, an aryl group, an alkenyl group, an aralkyl group, an alkoxy group, an alkylcarbonyloxy group, an alkylsulfonyloxy group, an alkyloxycarbonyl group, or an aryloxycarbonyl group. When a plurality of R's are present, they may be the same or different. When a plurality of R's are present, they may together form a ring. R is preferably a hydrogen atom. a represents an integer of from 1 to 3. b represents and integer of 0 to (5-a).

Examples of the repeating unit having the acid group are shown below. In the following formulas, a represents 1 or 2.

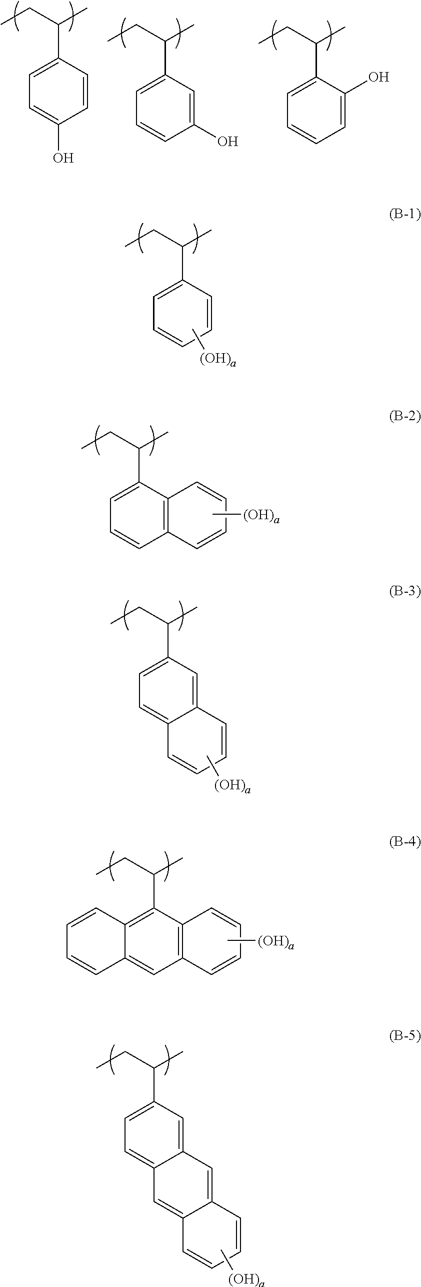

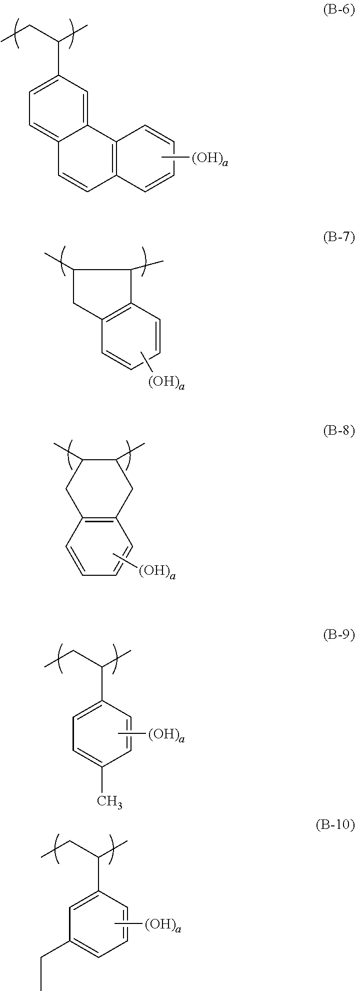

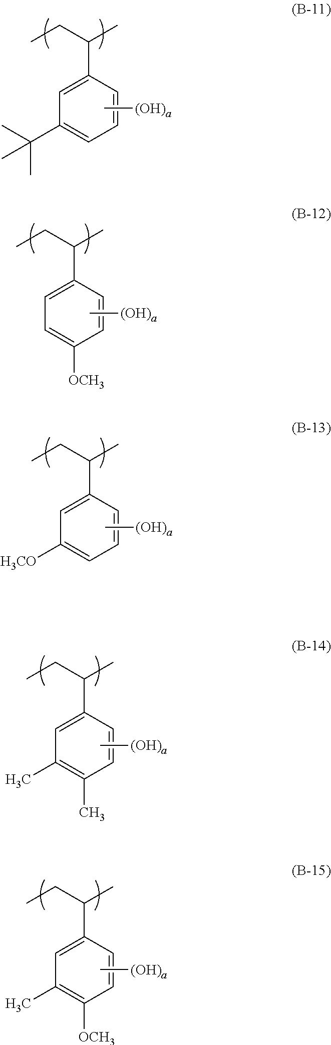

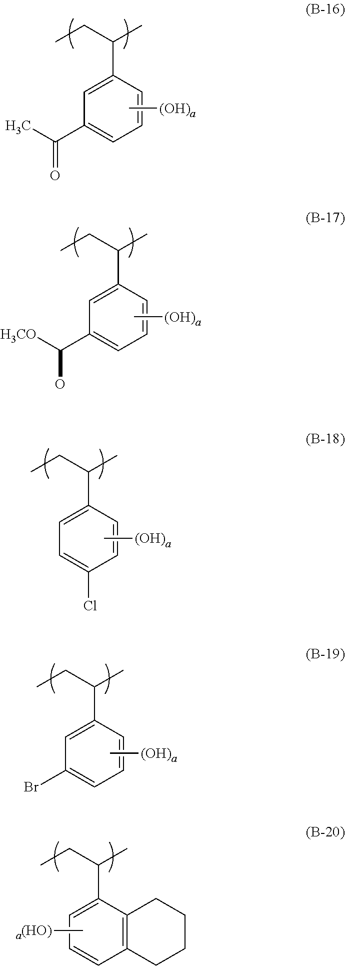

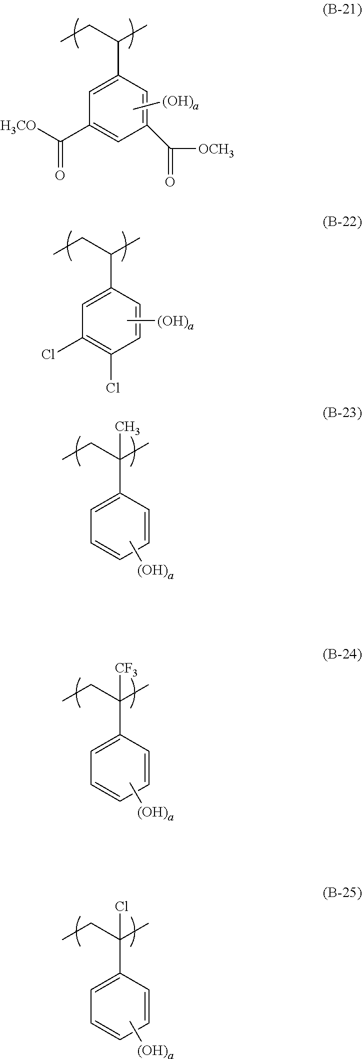

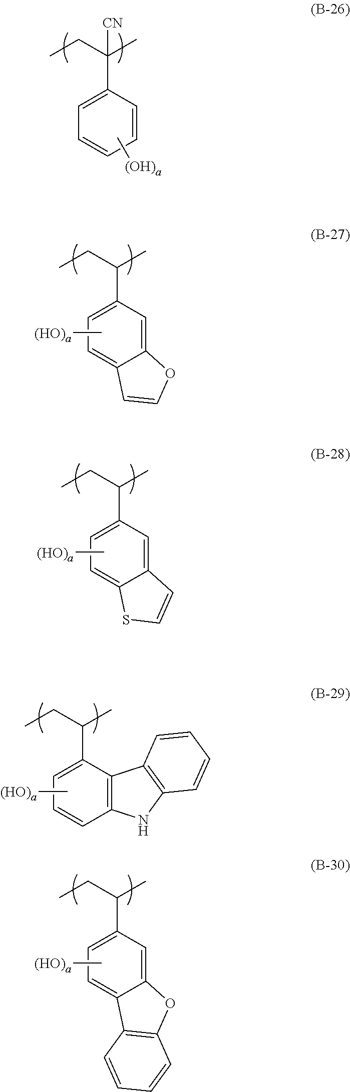

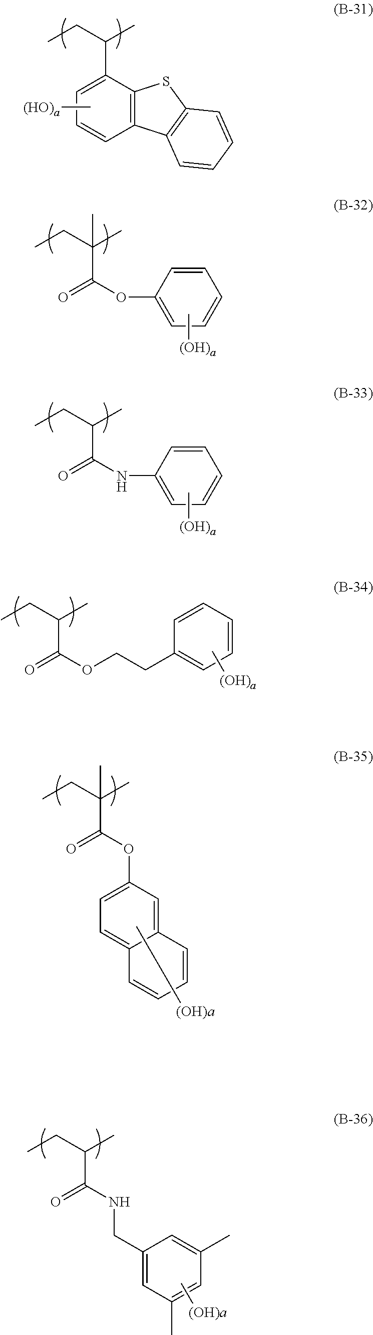

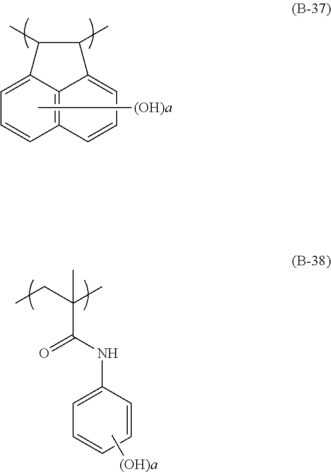

Among the above repeating units, repeating units specifically described below are preferred. In the following formulas, R represents a hydrogen atom or a methyl group, and a represents 2 or 3.

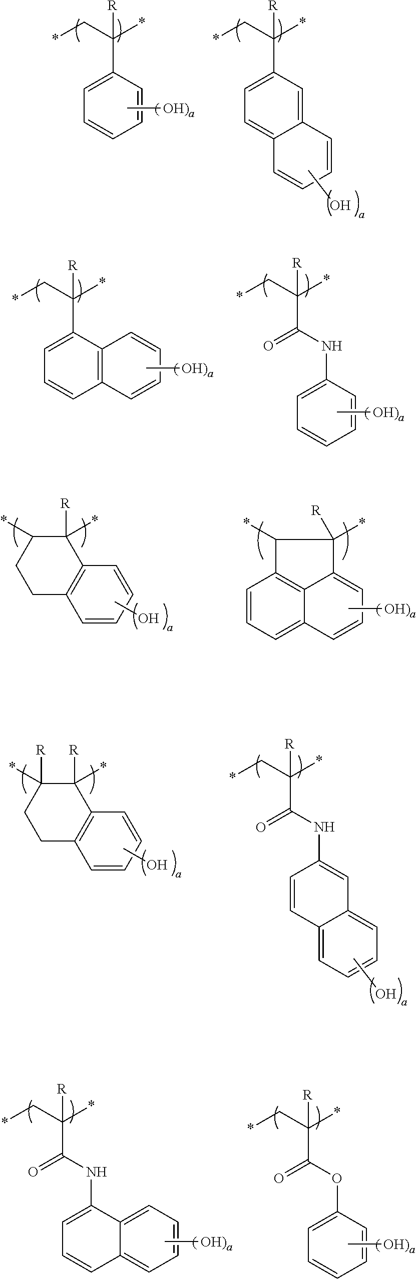

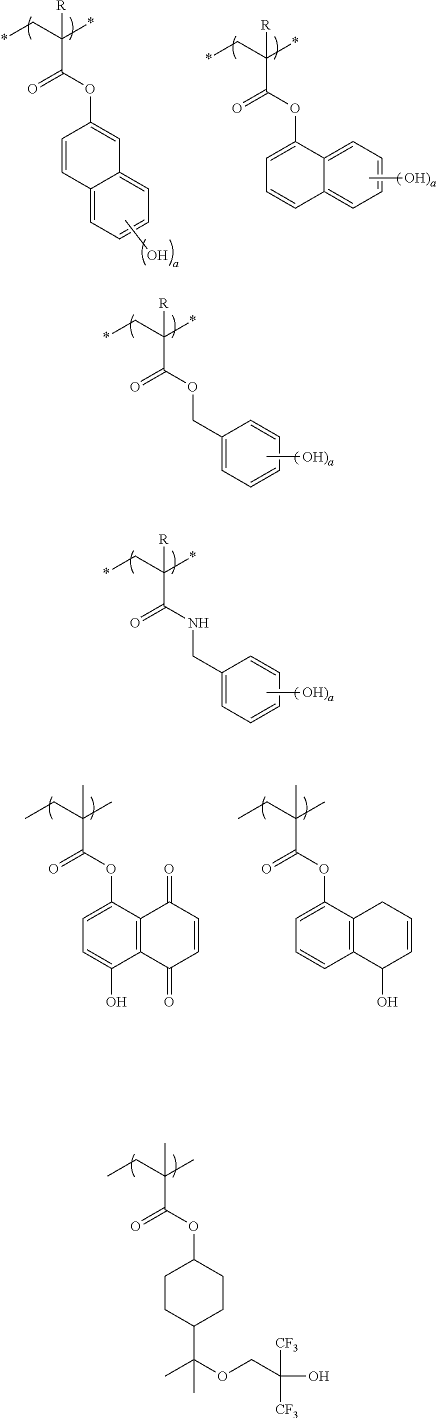

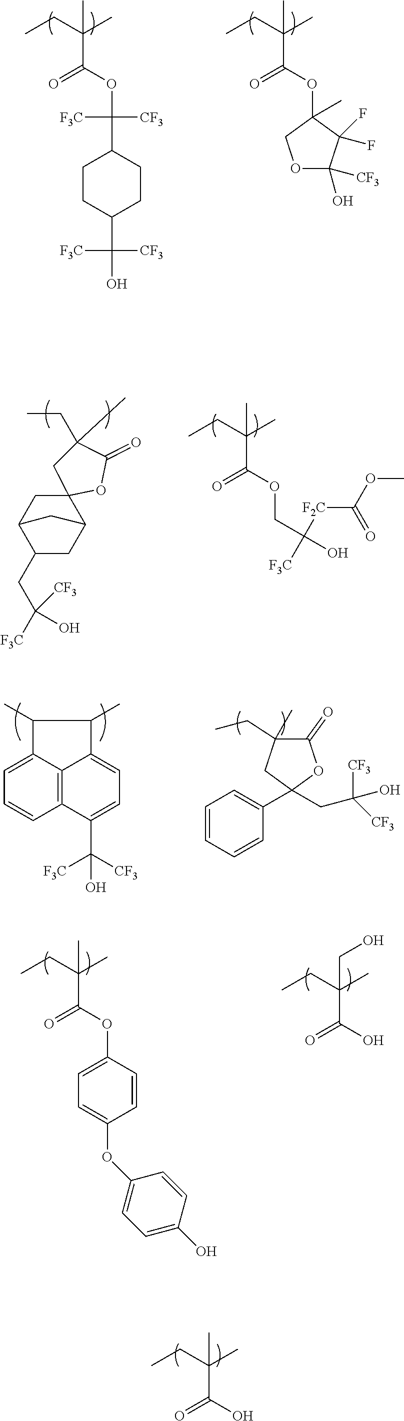

The content of the repeating unit having the acid group with respect to the total amount of the repeating units in the resin (A) is preferably 10% by mole or more and more preferably 15% by mole or more. The upper limit of the content with respect to the total amount of the repeating units in the resin (A) is preferably 70% by mole or less, more preferably 65% by mole or less, and still more preferably 60% by mole or less.

### (Repeating Unit Having No Acid-Decomposable Group and No Acid Group but Having Fluorine Atom, Bromine Atom, or Iodine Atom)

The resin (A) may have, in addition to the above-described <repeating unit having the acid-decomposable group> and the above-described <repeating unit having the acid group>, a repeating unit having no acid-decomposable group and no acid group but having a fluorine atom, a bromine atom, or an iodine atom (this repeating unit is hereinafter referred to also as a unit X). Preferably, the <repeating unit having no acid-decomposable group and no acid group but having a fluorine atom, a bromine atom, or an iodine atom> differs from other types of repeating units belonging to the group A such as the <repeating unit having a lactone group, a sultone group, or a carbonate group> described later and the <repeating unit having a photoacid generating group> described later.

The unit X is preferably a repeating unit represented by formula (C).

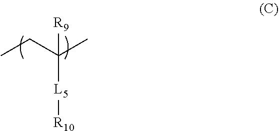

L₅ represents a single bond or an ester group. R₉ represents a hydrogen atom or an alkyl group optionally having a fluorine atom or an iodine atom. R₁₀ represents a hydrogen atom, an alkyl group optionally having a fluorine atom or an iodine atom, a cycloalkyl group optionally having a fluorine atom or an iodine atom, an aryl group optionally having a fluorine atom or an iodine atom, or a combination thereof.

Examples of the repeating unit having a fluorine atom or an iodine atom are shown below.

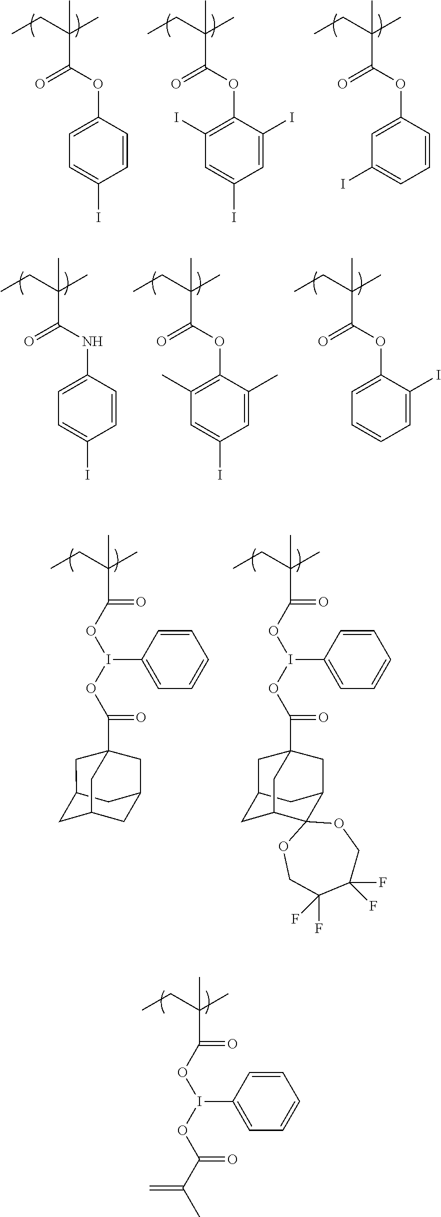

The content of the unit X with respect to the total amount of the repeating units in the resin (A) is preferably 0% by mole or more, more preferably 5% by mole or more, and still more preferably 10% by mole or more. The upper limit of the content of the unit X with respect to the total amount of the repeating units in the resin (A) is preferably 50% by mole or less, more preferably 45% by mole or less, and still more preferably 40% by mole or less.

Among the repeating units in the resin (A), the total amount of repeating units including at least one of a fluorine atom, a bromine atom, or an iodine atom with respect to the total amount of the repeating units in the resin (A) is preferably 10% by mole or more, more preferably 20% by mole or more, still more preferably 30% by mole or more, and particularly preferably 40% by mole or more. No particular limitation is imposed on the upper limit of the total amount, but the amount with respect to the total amount of the repeating units in the resin (A) is, for example, 100% by mole or less.

Examples of the repeating units including at least one of a fluorine atom, a bromine atom, or an iodine atom include: a repeating unit having a fluorine atom, a bromine atom, or an iodine atom and having the acid-decomposable group; a repeating unit having a fluorine atom, a bromine atom, or an iodine atom and having the acid group; and a repeating unit having a fluorine atom, a bromine atom, or an iodine atom.

(Repeating unit having lactone group, sultone group, or carbonate group)

The resin (A) may have a repeating unit having at least one selected from the group consisting of lactone groups, sultone groups, and carbonate groups (this repeating unit is hereafter referred to also as a “unit Y”).

It is also preferable that the unit Y does not have a hydroxy group and an acid group such as a hexafluoroisopropanol group.

The lactone or sultone group may be any lactone or sultone group so long as it has a lactone or sultone structure. The lactone or sultone structure is preferably a 5- to 7-membered lactone or sultone structure. In particular, a 5- to 7-membered lactone structure with another ring structure fused thereto to form a bicyclo or spiro structure or a 5- to 7-membered sultone structure with another ring structure fused thereto to form a bicyclo or spiro structure is more preferred.

Preferably, the resin (A) has a repeating unit having a lactone or sultone group formed by removing at least one hydrogen atom from a ring member atom of a lactone structure represented by any of the following formulas (LC1-1) to (LC1-21) or a sultone structure represented by any of the following formulas (SL1-1) to (SL1-3).

The lactone or sultone group may be bonded directly to the main chain. For example, a ring member atom of the lactone or sultone group may be included in the main chain of the resin (A).

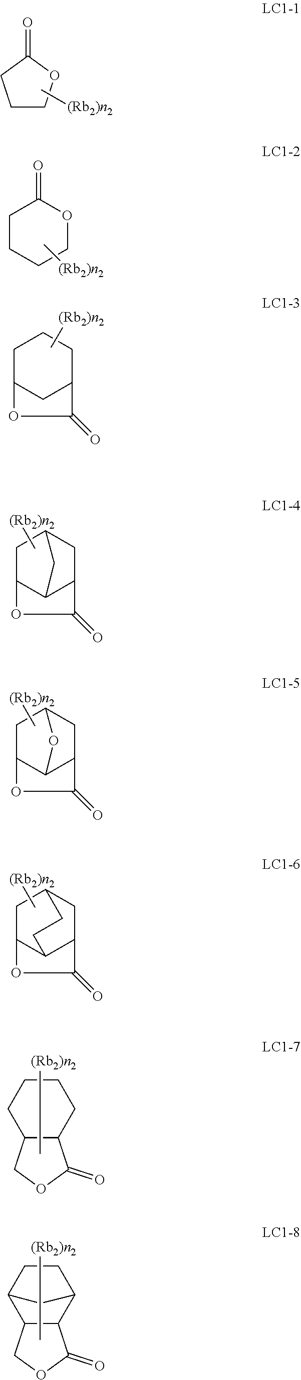

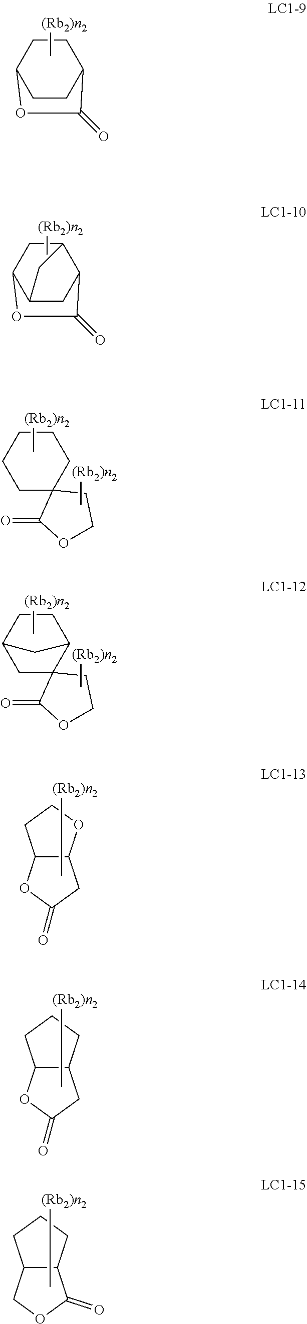

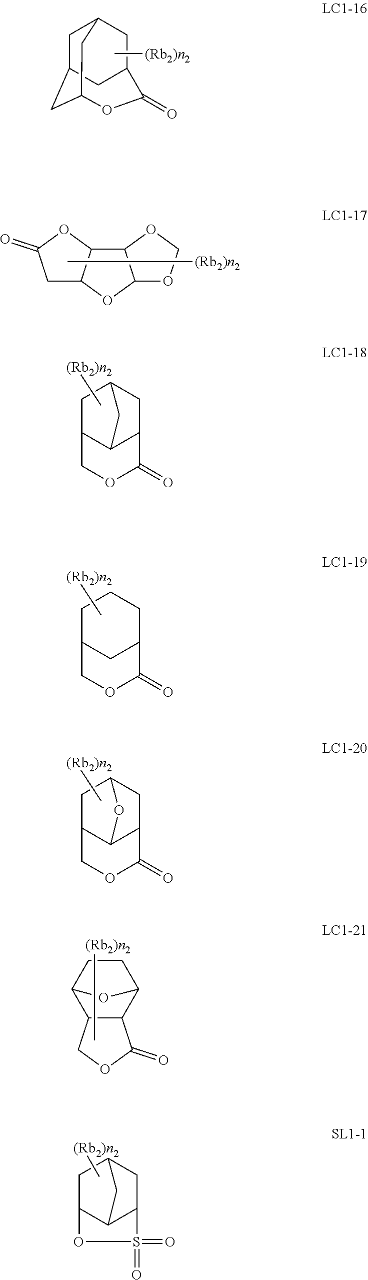

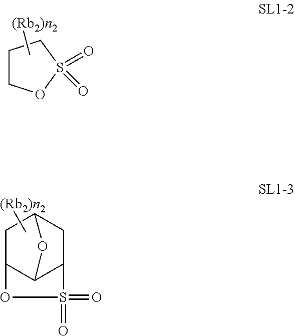

Each of the lactone and sultone structures may have a substituent (Rb₂). Preferred examples of the substituent (Rb2) include alkyl groups having 1 to 8 carbon atoms, cycloalkyl groups having 4 to 7 carbon atoms, alkoxy groups having 1 to 8 carbon atoms, alkoxycarbonyl groups having 1 to 8 carbon atoms, a carboxy group, halogen atoms, a cyano group, and acid-decomposable groups. n₂ represents an integer of from 0 to 4. A plurality of Rb₂'s present when n₂ is 2 or more may be different from each other, and the plurality of Rb₂'s present may be bonded together to form a ring.

Examples of the repeating unit having a group including the lactone structure represented by any of formulas (LC1-1) to (LC1-21) or the sultone structure represented by any of formulas (SL1-1) to (SL1-3) include a repeating unit represented by the following formula (AI).

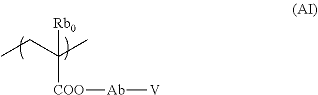

In formula (AI), Rb₀ represents a hydrogen atom, a halogen atom, or an alkyl group having 1 to 4 carbon atoms. The alkyl group represented by Rb₀ may have a substituent, and preferred examples of the substituent include a hydroxy group and halogen atoms.

Examples of the halogen atom represented by Rb₀ include a fluorine atom, a chlorine atom, a bromine atom, and an iodine atom. Rb₀ is preferably a hydrogen atom or a methyl group.

Ab represents a single bond, an alkylene group, a divalent linking group having a monocyclic or polycyclic alicyclic hydrocarbon structure, an ether group, an ester group, a carbonyl group, a carboxy group, or a divalent group formed by combining any of the above groups. In particular, Ab is preferably a single bond or a linking group represented by -Ab₁-CO₂—. Ab₁ is a linear or branched alkylene group or a monocyclic or polycyclic cycloalkylene group and is preferably a methylene group, an ethylene group, a cyclohexylene group, an adamantylene group, or a norbornylene group.

V represents a group formed by removing one hydrogen atom from a ring member atom in the lactone structure represented by any of formulas (LC1-1) to (LC1-21) or a group formed by removing one hydrogen atom from a ring member atom in the sultone structure represented by any of formulas (SL1-1) to (SL1-3).

When the repeating unit having the lactone or sultone group has optical isomers, any of the optical isomers may be used. One optical isomer may be used alone, or a mixture of a plurality of optical isomers may be used. When one optical isomer is mainly used, the optical purity (ee) thereof is preferably 90 or more and more preferably 95 or more.

The carbonate group is preferably a cyclic carbonate group.

The repeating unit having a cyclic carbonate group is preferably a repeating unit represented by the following formula (A-1).

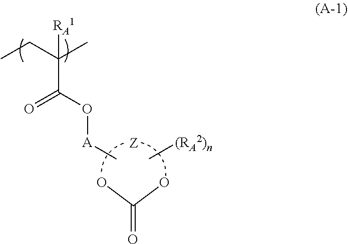

In formula (A-1), R_{A}¹ represents a hydrogen atom, a halogen atom, or a monovalent organic group (preferably a methyl group). n represents an integer of 0 or more. R_{A}² represents a substituent. A plurality of R_{A}²'s present when n is 2 or more may be the same or different. A represents a single bond or a divalent linking group. The divalent linking group is preferably an alkylene group, a divalent linking group having a monocyclic or polycyclic alicyclic hydrocarbon structure, an ether group, an ester group, a carbonyl group, a carboxy group, or a divalent group formed by combining any of them. Z represents an atomic group forming a monocyclic or polycyclic ring together with a group represented by —O—CO—O— in formula (A-1).

Examples of the unit Y are shown below.

(In the following formulas, Rx is H CH₃, CH₂OH, or CF₃.)

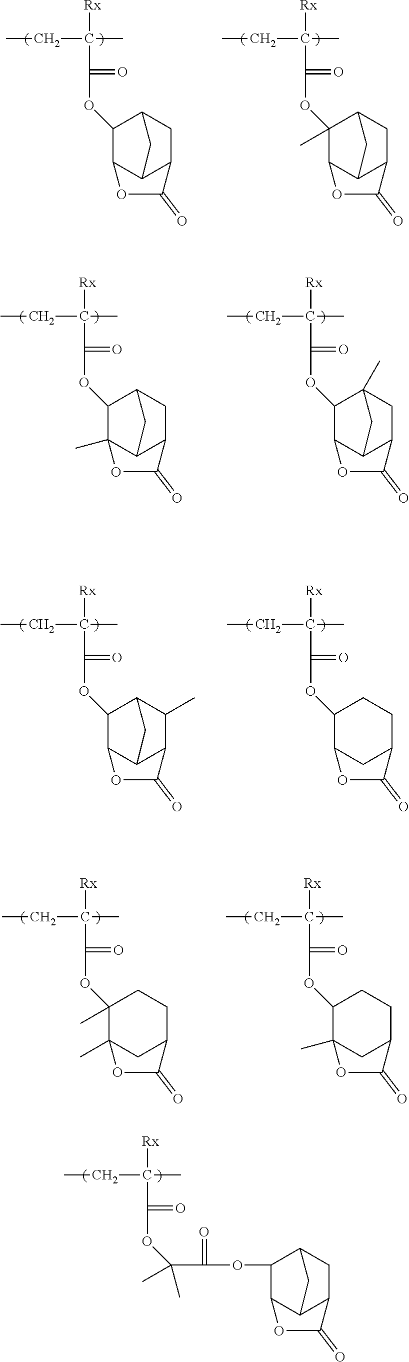

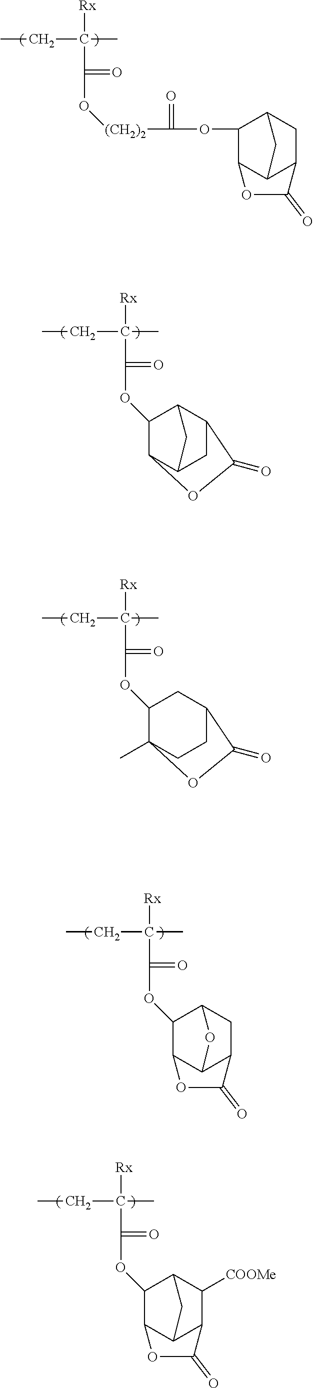

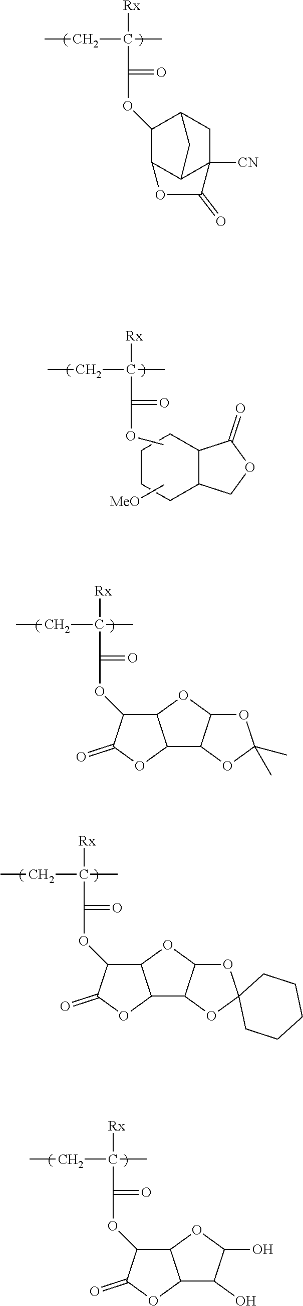

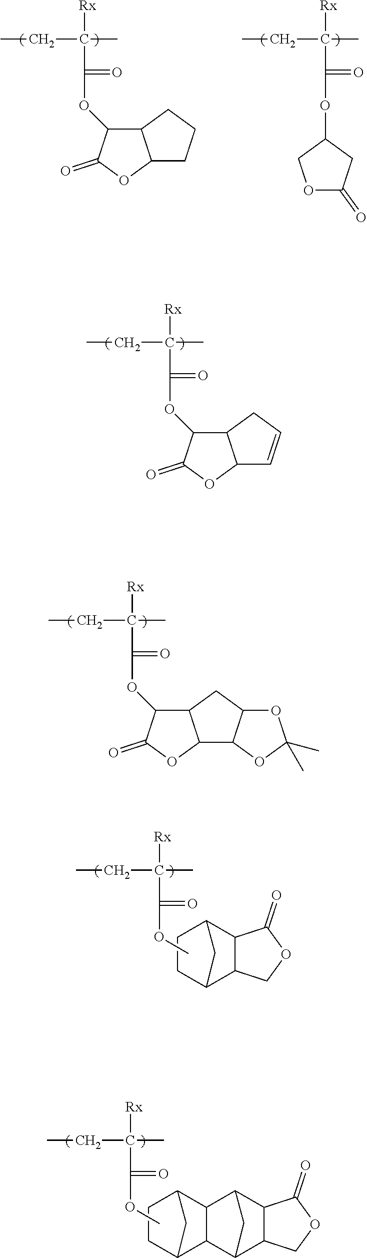

(In the following formulas, Rx is H CH₃, CH₂OH, or CF₃.)

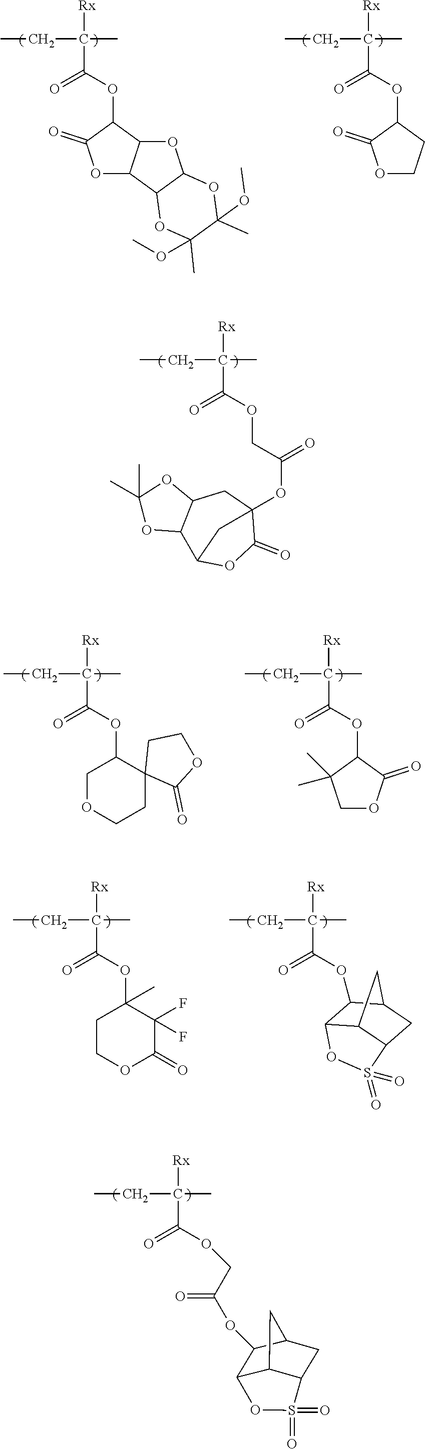

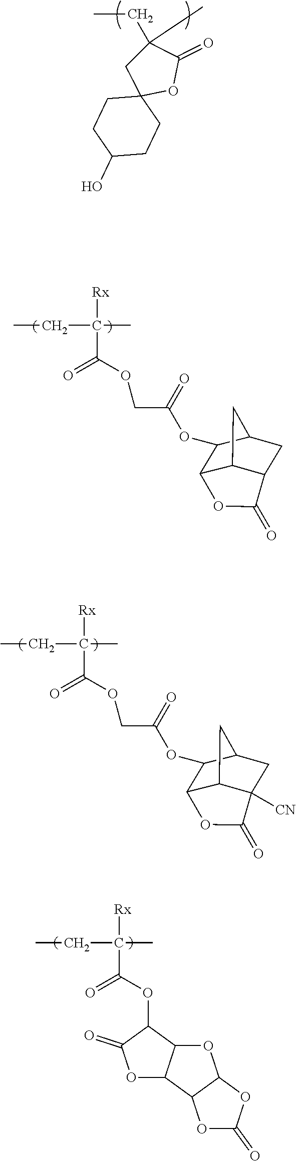

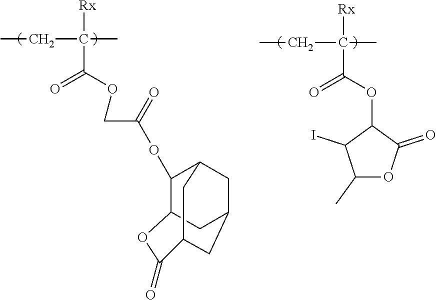

(In the following formulas, Rx is H CH₃, CH₂OH, or CF₃.)

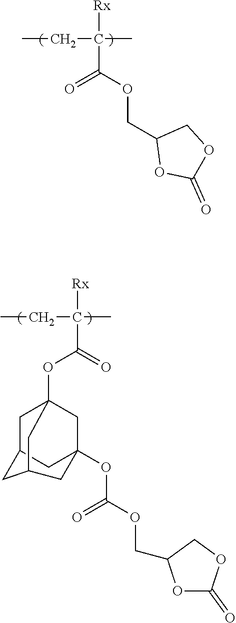

The content of the unit Y with respect to the total amount of the repeating units in the resin (A) is preferably 1% by mole or more and more preferably 10% by mole or more. The upper limit of the content of the unit Y with respect to the total amount of the repeating units in the resin (A) is preferably 85% by mole or less, more preferably 80% by mole or less, still more preferably 70% by mole or less, and particularly preferably 60% by mole or less.

### (Repeating Unit Having Photoacid Generating Group)

The resin (A) may include a repeating unit that is different from those described above and has a group that generates an acid when irradiated with actinic rays or radiation (this group is hereinafter referred to also as a “photoacid generating group”).

Examples of the repeating unit having the photoacid generating group include a repeating unit represented by formula (4).

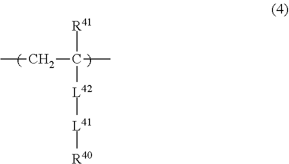

R⁴¹ represents a hydrogen atom or a methyl group. L⁴¹ represent a single bond or a divalent linking group. L⁴² represents a divalent linking group. R⁴⁰ represents a structural moiety that is decomposed when irradiated with actinic rays or radiation and thereby generates an acid on a side chain.

Examples of the repeating unit having the photoacid generating group are shown below.

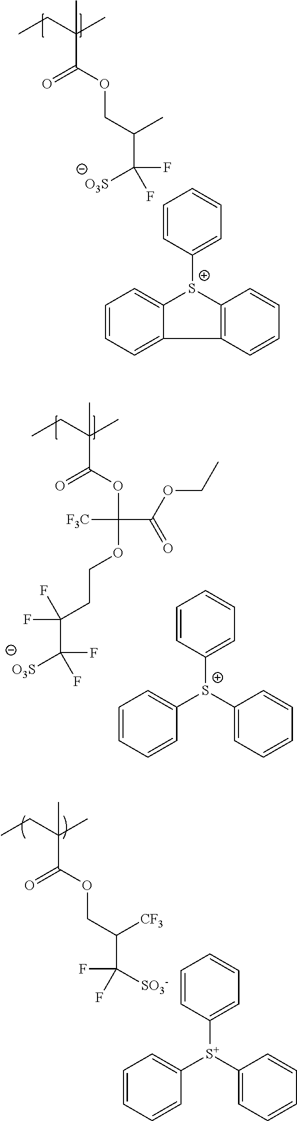

Other examples of the repeating unit represented by formula (4) include repeating units described in paragraphs [0094] to [0105] of JP2014-041327A and repeating units described in paragraph [0094] of WO2018/193954A.

The content of the repeating unit having the photoacid generating group with respect to the total amount of the repeating units in the resin (A) is preferably 1% by mole or more and more preferably 5% by mole or more. The upper limit of the content with respect to the total amount of the repeating units in the resin (A) is preferably 40% by mole or less, more preferably 35% by mole or less, and still more preferably 30% by mole or less.

### (Repeating Unit Represented by Formula (V-1) or Formula (V-2))

The resin (A) may have a repeating unit represented by formula (V-1) or (V-2) below.

Preferably, the repeating unit represented by the following formula (V-1) or (V-2) differs from the repeating units described above.

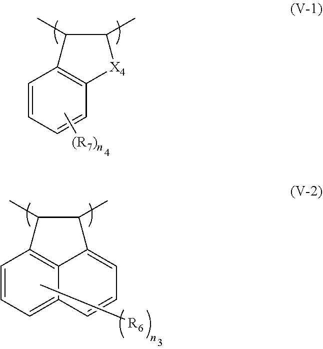

In these formulas, R⁶ and R₇ each independently represent a hydrogen atom, a hydroxy group, an alkyl group, an alkoxy group, an acyloxy group, a cyano group, a nitro group, an amino group, a halogen atom, an ester group (—OCOR or —COOR: R represents an alkyl group having 1 to 6 carbon atoms or a fluorinated alkyl group having 1 to 6 carbon atoms), or a carboxy group. The alkyl group is preferably a linear, branched, or cyclic alkyl group having 1 to 10 carbon atoms.
n₃ represents an integer of from 0 to 6.
n₄ represents an integer of from 0 to 4.
X₄ is a methylene group, an oxygen atom, or a sulfur atom.

Examples of the repeating unit represented by formula (V-1) or (V-2) are shown below.

Examples of the repeating unit represented by formula (V-1) or (V-2) include repeating units described in paragraph [0100] of WO2018/193954A.

### (Repeating Unit for Reducing Mobility of Main Chain)

The higher the glass transition temperature (Tg) of the resin (A), the better because excessive diffusion of the acid generated or pattern collapse during development can be prevented. The Tg is preferably higher than 90° C., more preferably higher than 100° C., still more preferably higher than 110° C., and particularly preferably higher than 125° C. The Tg is preferably 400° C. or lower and more preferably 350° C. or lower because the rate of dissolution in a developer is high.

In the present specification, the glass transition temperature (Tg) of a polymer such as the resin (A) (hereinafter referred to as the “Tg of a repeating unit”) is computed by the following method. First, the Tg of each of the homopolymers formed from the respective repeating units included in the polymer is computed by the Bicerano method. Next, the mass ratios (%) of the repeating units with respect to the total mass of the repeating units in the polymer are computed. Next, the Tg of each repeating unit at the corresponding mass ratio is computed using the Fox formula (described, for example, in Materials Letters 62 (2008) 3152), and the computed Tg's are summed to obtain the Tg (° C.) of the polymer.

The Bicerano method is described in Prediction of polymer properties, Marcel Dekker Inc, New York (1993). The computation of Tg by the Bicerano method can be performed using software for estimating physical properties of a polymer, MDL Polymer (MDL Information Systems, Inc.).

To increase the Tg of the resin (A) (to increase the Tg to preferably higher than 90° C.), it is preferable to reduce the mobility of the main chain of the resin (A). Examples of a method for reducing the mobility of the main chain of the resin (A) include methods (a) to (e) described below.
(a) Introduction of a bulky substituent into the main chain.
(b) Introduction of a plurality of substituents into the main chain.
(c) Introduction of a substituent that induces the interaction between molecules of the resin (A) into the vicinities of their main chains.
(d) Formation of the main chain having a ring structure.
(e) Linkage of a ring structure to the main chain

Preferably, the resin (A) has a repeating unit whose homopolymer has a Tg of 130° C. or higher.

No particular limitation is imposed on the type of repeating unit whose homopolymer has a Tg of 130° C. or higher, and any repeating unit can be used so long as the Tg of the homopolymer computed by the Bicerano method is 130° C. or higher. With repeating units represented by formulas (A) to (E) described below, homopolymers formed from the repeating units can have a Tg of 130° C. or higher, but this depends on the types of functional groups in the repeating units.

One specific example of means for achieving the method (a) is a method in which the repeating unit represented by formula (A) is introduced into the resin (A).

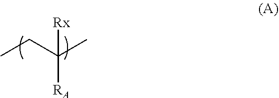

In Formula (A), R_{A} represents a group including a polycyclic structure. Rx represents a hydrogen atom, a methyl group, or an ethyl group. The group including the polycyclic structure is a group including a plurality of ring structures, and the plurality of ring structures may or may not be fused.

Specific examples of the repeating unit represented by formula (A) include those described in paragraphs [0107] to [0119] of WO2018/193954A.

One specific example of means for achieving the method (b) is a method in which the repeating unit represented by formula (B) is introduced into the resin (A).

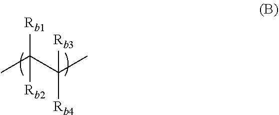

In formula (B), R_{b1} to R_{b4} each independently represent a hydrogen atom or an organic group, and at least two selected from the group consisting of R_{b1} to R_{b4} each represent an organic group.

When at least one of the organic groups is a group whose ring structure is linked directly to the main chain of the repeating unit, no particular limitation is imposed on the types of other organic groups.

When each of the organic groups is not a group whose ring structure is linked directly to the main chain of the repeating unit, at least two of the organic groups are each a substituent in which the number of constituent atoms excluding hydrogen atoms is 3 or more.

Specific examples of the repeating unit represented by formula (B) include those described in paragraphs [0113] to [0115] of WO2018/193954A.

One specific example of means for achieving the method (c) is a method in which the repeating unit represented by formula (C) is introduced into the resin (A).

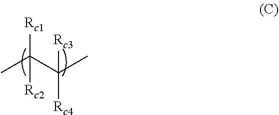

In formula (C), R_{c1} to R_{c4} each independently represent a hydrogen atom or an organic group, and at least one of R_{c1}, R_{c2}, R_{c3}, or R_{c4} is a group including a hydrogen-bonding hydrogen atom at a position within 3 atoms from a carbon atom in the main chain. In particular, it is preferable that the hydrogen-bonding hydrogen atom is present at a position within two atoms (at a position closer to the main chain) in order to induce the interaction between the main chains of molecules of the resin (A).

Specific examples of the repeating unit represented by formula (C) include those described in paragraphs [0119] to [0121] of WO2018/193954A.

One specific example of means for achieving the method (d) is a method in which the repeating unit represented by formula (D) is introduced into the resin (A).

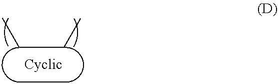

In formula D, “Cyclic” represents a group having a ring structure forming the main chain. No particular limitation is imposed on the number of atoms forming the ring.

Specific examples of the repeating unit represented by formula (D) include those described in paragraphs [0126] to [0127] of WO2018/193954A.

One specific example of means for achieving the method (e) is a method in which the repeating unit represented by formula (E) is introduced into the resin (A).

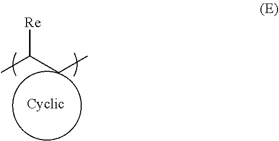

In formula (E), Re's each independently represent a hydrogen atom or an organic group. Examples of the organic group include alkyl groups, cycloalkyl groups, aryl groups, aralkyl groups, and alkenyl groups, each of which may have a substituent.

“Cyclic” is a cyclic group including a carbon atom included in the main chain. No particular limitation is imposed on the number of atoms included in the cyclic group.

Specific examples of the repeating unit represented by formula (E) include those described in paragraphs [0131] to [0133] of WO2018/193954A.

(Repeating Unit Having at Least One Group Selected from Group Consisting of Lactone Groups, Sultone Groups, Carbonate Groups, Hydroxy Group, Cyano Group, and Alkali-Soluble Groups)

The resin (A) may have a repeating unit having at least one group selected from the group consisting of lactone groups, sultone groups, carbonate groups, a hydroxy group, a cyano group, and alkali-soluble groups.

The repeating unit having a lactone group, a sultone group, or a carbonate group and included in the resin (A) may be any of the repeating units described above for the <repeating unit having a lactone group, a sultone group, or a carbonate group>. A preferred content of the repeating unit is also as described above for the <repeating unit having a lactone group, a sultone group, or a carbonate group>.

The resin (A) may have a repeating unit having a hydroxy group or a cyano group. In this case, the adhesiveness to a substrate and the affinity for a developer are improved.

The repeating unit having a hydroxy group or a cyano group is preferably a repeating unit having an alicyclic hydrocarbon structure substituted with a hydroxy group or a cyano group.

Preferably, the repeating unit having a hydroxy group or a cyano group has no acid-decomposable group. Examples of the repeating unit having a hydroxy group or a cyano group include those described in paragraphs [0081] to [0084] of JP2014-098921A.

The resin (A) may have a repeating unit having an alkali-soluble group.

Examples of the alkali-soluble group include a carboxy group, a sulfonamido group, a sulfonylimido group, a bissulfonylimido group, and aliphatic alcohol groups substituted with an electron-withdrawing group at the α-position (e.g., a hexafluoroisopropanol group), and the alkali-soluble group is preferably a carboxy group. When the resin (A) includes the repeating unit having an alkali-soluble group, resolution in contact hole applications is increased. Examples of the repeating unit having an alkali-soluble group include those described in paragraphs [0085] and [0086] of JP2014-098921A.

### (Repeating Unit Having Alicyclic Hydrocarbon Structure and Exhibiting No Acid Decomposability)

The resin (A) may have a repeating unit having an alicyclic hydrocarbon structure and exhibiting no acid decomposability. In this case, elution of a low-molecular weight component from the resist film to an immersion liquid during liquid immersion exposure can be reduced. Examples of such a repeating unit include repeating units derived from 1-adamantyl (meth)acrylate, diamantyl (meth)acrylate, tricyclodecanyl (meth)acrylate, and cyclohexyl (meth)acrylate.

### (Repeating Unit Represented by Formula (III) and Having No Hydroxy Group and No Cyano Group)

The resin (A) may have a repeating unit represented by formula (III) and having no hydroxy group and no cyano group.

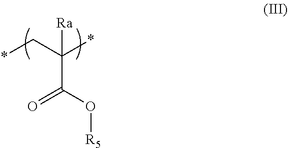

In formula (III), R₅ represents a hydrocarbon group having at least one ring structure and having no hydroxy group and no cyano group.

Ra represents a hydrogen atom, an alkyl group, or a —CH₂—O—Ra₂ group. In this formula, Ra₂ represents a hydrogen atom, an alkyl group, or an acyl group.

Examples of the repeating unit represented by formula (III) and having no hydroxy group and no cyano group include those described in paragraphs [0087] to [0094] of JP2014-098921A.

### (Additional Repeating Units)

The resin (A) may further have a repeating unit other than the repeating units described above.

For example, the resin (A) may have a repeating unit selected from the group consisting of a repeating unit having an oxathiane ring group, a repeating unit having an oxazolone ring group, a repeating unit having a dioxane ring group, and a repeating unit having a hydantoin ring group.

Examples of such a repeating unit are shown below.

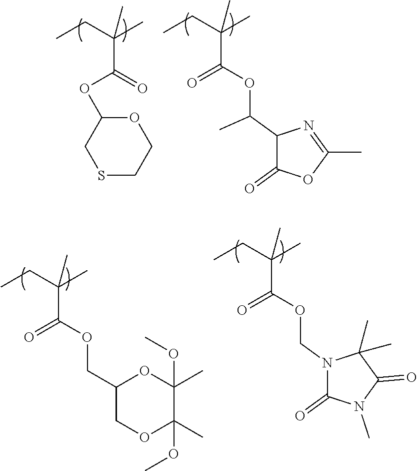

The resin (A) may have, in addition to the repeating units described above, various repeating units for the purpose of controlling dry etching resistance, suitability for a standard developer, adhesiveness to a substrate, a resist profile, resolution, heat resistance, sensitivity, etc.

Preferably, in the resin (A) (particularly when the composition is used as an actinic ray-sensitive or radiation-sensitive resin composition for ArF light), all the repeating units are composed of repeating units derived from compounds having an ethylenically unsaturated bond. In particular, it is also preferable that all the repeating units are composed of (meth)acrylate-based repeating units. In the resin (A) used in this case, all the repeating units may be methacrylate-based repeating units, or all the repeating units may be acrylate-based repeating units. Alternatively, the repeating units may each be a methacrylate-based repeating unit or an acrylate-based repeating unit. It is preferable that the content of the acrylate-based repeating units with respect to the total amount of the repeating units is 50% by mole or less.

The resin (A) can be synthesized by a routine method (for example, radical polymerization).

The weight average molecular weight of the resin (A) that is determined as a polystyrene-equivalent value by the GPC method is preferably 30,000 or less, more preferably 1,000 to 30,000, still more preferably 3,000 to 30,000, and particularly preferably 5,000 to 15,000.

The dispersity (molecular weight distribution) of the resin (A) is preferably 1 to 5, more preferably 1 to 3, still more preferably 1.2 to 3.0, and particularly preferably 1.2 to 2.0. The smaller the dispersity, the better the resolution and the resist profile, and the smoother the side surfaces of the resist pattern, so that better roughness quality is obtained.

In the composition of the invention, the content of the resin (A) with respect to the total amount of the solids in the composition is preferably 40.0 to 99.9% by mass and more preferably 60.0 to 90.0% by mass.

One resin (A) may be used alone, or a combination of a plurality of resins (A) may be used.

### <Photoacid Generator>

The composition of the invention may include a photoacid generator (B) that does not correspond to the compound (I).

The photoacid generator (B) is a compound that generates an acid necessary for the reaction of the resin in the exposed portions.

The photoacid generator (B) may be in the form of a low-molecular weight compound or may be in the form in which the photoacid generator (B) is incorporated into part of a polymer (e.g., the resin (A) described later). A combination of the form of a low-molecular-weight compound and the form in which the photoacid generator (B) is incorporated into part of a polymer (e.g., the resin (A) described later) may also be used.

When the photoacid generator (B) is in the form of a low-molecular weight compound, the molecular weight of the photoacid generator is preferably 3000 or less, more preferably 2000 or less, and still more preferably 1000 or less. No particular limitation is imposed on the lower limit of the molecular weight, but the molecular weight is 100 or more.

When the photoacid generator (B) is in the form in which the photoacid generator (B) is incorporated into part of a polymer, the photoacid generator (B) may be incorporated into part of the resin (A) or into a resin different from the resin (A).

In the present invention, it is preferable that the photoacid generator (B) is in the form of a low-molecular weight compound.

The photoacid generator (B) is, for example, a compound (onium salt) represented by “MX” and is preferably a compound that generates an organic acid upon exposure to light.

Examples of the organic acid include sulfonic acids (such as aliphatic sulfonic acids, aromatic sulfonic acids, and camphorsulfonic acid), carboxylic acids (such as aliphatic carboxylic acids, aromatic carboxylic acids, and aralkyl carboxylic acids), carbonylsulfonylimidic acid, bis(alkylsulfonyl)imidic acids, and tris(alkylsulfonyl)methide acids.

In the compound represented by “M⁺X⁻,” M⁺ represents an organic cation.

No particular limitation is imposed on the organic cation. The valence of the organic cation may be 1 or 2 or more.

No particular limitation is imposed on the organic cation, but the organic cation is preferably the cation represented by formula (ZaI) above (hereinafter referred to also as a “cation (ZaI)”) or the cation represented by formula (ZaII) above (hereinafter referred to also as a “cation (ZaII)”).

In the compound represented by “M⁺X⁻,” X⁻ represents an organic anion.

No particular limitation is imposed on the organic anion, and examples thereof include monovalent organic anions and divalent and higher valent organic anions.

The organic anion is preferably an anion whose ability to cause a nucleophilic reaction is very low and is more preferably a non-nucleophilic anion.

Examples of the non-nucleophilic anion include sulfonate anions (such as aliphatic sulfonate anions, aromatic sulfonate anions, and a camphorsulfonate anion), carboxylate anions (such as aliphatic carboxylate anions, aromatic carboxylate anions, and aralkyl carboxylate anions), sulfonylimide anions, bis(alkylsulfonyl)imide anions, and tris(alkylsulfonyl)methide anions.

In the aliphatic sulfonate anions and the aliphatic carboxylate anions, the aliphatic moiety may be a linear or branched alkyl group or a cycloalkyl group and is preferably a linear or branched alkyl group having 1 to 30 carbon atoms or a cycloalkyl group having 3 to 30 carbon atoms.

The alkyl group may be, for example, a fluoroalkyl group (which may have a substituent other than a fluorine atom or may be a perfluoroalkyl group).

In the aromatic sulfonate anions and the aromatic carboxylate anions, the aryl group is preferably an aryl group having 6 to 14 carbon atoms such as a phenyl group, a tolyl group, or a naphthyl group.

The above-described alkyl, cycloalkyl, and aryl groups may each have a substituent. No particular limitation is imposed on the substituent, and examples thereof include a nitro group, halogen atoms such as a fluorine atom and a chlorine atom, a carboxy group, a hydroxy group, an amino group, a cyano group, alkoxy groups (having preferably 1 to 15 carbon atoms), alkyl groups (having preferably 1 to 10 carbon atoms), cycloalkyl groups (having preferably 3 to 15 carbon atoms), aryl groups (having preferably 6 to 14 carbon atoms), alkoxycarbonyl groups (having preferably 2 to 7 carbon atoms), acyl groups (having preferably 2 to 12 carbon atoms), alkoxycarbonyloxy groups (having preferably 2 to 7 carbon atoms), alkylthio groups (having preferably 1 to 15 carbon atoms), alkylsulfonyl groups (having preferably 1 to 15 carbon atoms), alkyliminosulfonyl groups (having preferably 1 to 15 carbon atoms), and aryloxysulfonyl groups (having preferably 6 to 20 carbon atoms).

In the aralkyl carboxylate anions, the aralkyl group is preferably an aralkyl group having 7 to 14 carbon atoms.

Examples of the aralkyl group having 7 to 14 carbon atoms include a benzyl group, a phenethyl group, a naphthylmethyl group, a naphthylethyl group, and a naphthylbutyl group.

Examples of the sulfonylimide anion include a saccharin anion.

In the bis(alkylsulfonyl)imide anions and the tris(alkylsulfonyl)methide anions, the alkyl group is preferably an alkyl group having 1 to 5 carbon atoms. These alkyl groups may have a substituent, and examples of the substituent include halogen atoms, alkyl groups substituted with halogen atoms, alkoxy groups, alkylthio groups, alkyloxysulfonyl groups, aryloxysulfonyl groups, and cycloalkylaryloxysulfonyl groups. The substituent is preferably a fluorine atom or an alkyl group substituted with a fluorine atom.

In the bis(alkylsulfonyl)imide anions, the alkyl groups may be bonded together to form a ring structure. In this case, the strength of the acid increases.

Other examples of the non-nucleophilic anion include phosphorus fluoride (such as PF₆), boron fluoride (such as BF₄⁻), and antimony fluoride (such as SbF₆⁻).

The non-nucleophilic anion is preferably an aliphatic sulfonate anion substituted with a fluorine atom at least at the α-position of the sulfonic acid, an aromatic sulfonate anion substituted with a fluorine atom or a fluorine atom-containing group, a bis(alkylsulfonyl)imide anion in which an alkyl group is substituted with a fluorine atom, or a tris(alkylsulfonyl)methide anion in which an alkyl group is substituted with a fluorine atom. In particular, the non-nucleophilic anion is more preferably a perfluoroaliphatic sulfonate anion (having preferably 4 to 8 carbon atoms) or a benzenesulfonate anion having a fluorine atom and still more preferably a nonafluorobutanesulfonate anion, a perfluorooctanesulfonate anion, a pentafluorobenzenesulfonate anion, or a 3,5-bis(trifluoromethyl)benzenesulfonate anion.

The non-nucleophilic anion is also preferably an anion represented by the following formula (AN1).

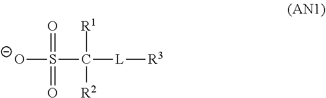

In formula (AN1), R¹ and R² each independently represent a hydrogen atom or a substituent.

No particular limitation is imposed on the substituent, but the substituent is preferably a group other than electron-withdrawing groups. Example of the group other than electron-withdrawing groups include hydrocarbon groups, a hydroxy group, oxyhydrocarbon groups, oxycarbonyl hydrocarbon groups, an amino group, hydrocarbon-substituted amino groups, and hydrocarbon-substituted amido groups.

Preferably, these groups other than electron-withdrawing groups are each independently —R′, —OH, —OR′, —OCOR′, —NH₂, —NR′₂, —NHR′, or —NHCOR′. R′ is a monovalent hydrocarbon group.

Examples of the monovalent hydrocarbon group represented by R′ include: linear or branched monovalent hydrocarbon groups including alkyl groups such as a methyl group, an ethyl group, a propyl group, and a butyl group, alkenyl groups such as an ethenyl group, a propenyl group, and a butenyl group, and alkynyl groups such as an ethynyl group, a propynyl group, and a butynyl group; monovalent alicyclic hydrocarbon groups including cycloalkyl groups such as a cyclopropyl group, a cyclobutyl group, a cyclopentyl group, a cyclohexyl group, a norbornyl group, and a adamantyl group and cycloalkenyl groups such as a cyclopropenyl group, a cyclobutenyl group, a cyclopentenyl group, and a norbornenyl group; and monovalent aromatic hydrocarbon groups including aryl groups such as a phenyl group, a tolyl group, a xylyl group, a mesityl group, a naphthyl group, a methylnaphthyl group, an anthryl group, and a methylanthryl group and aralkyl groups such as a benzyl group, a phenethyl group, a phenylpropyl group, a naphthylmethyl group, and an anthrylmethyl group.

In particular, it is preferable that R¹ and R² each independently represent a hydrocarbon group (preferably a cycloalkyl group) or a hydrogen atom.

L represents a divalent linking group.

When a plurality of L's are present, they may be the same or different.

Examples of the divalent linking group include —O—CO—O—, —COO—, —CONH—, —CO—, —O—, —S—, —SO—, —SO₂—, alkylene groups (having preferably 1 to 6 carbon atoms), cycloalkylene groups (having preferably 3 to 15 carbon atoms), alkenylene groups (having preferably 2 to 6 carbon atoms), and divalent linking groups formed by combining any of these groups. In particular, the divalent linking group is preferably —O—CO—O—, —COO—, —CONH—, —CO—, —O—, —SO₂—, —O—CO—O— alkylene group-, —COO-alkylene group-, or —CONH-alkylene group- and more preferably —O—CO—O—, —O—CO—O-alkylene group-, —COO—, —CONH—, —SO₂—, or —COO-alkylene group-.

Preferably, Lis, for example, a group represented by the following formula (AN1-1).

*^{a}—(CR^{2a}₂)_{X}-Q-(CR^{2b}₂)_{Y}—*^{b}(AN1-1)

In formula (AN1-1), *^{a} represents a bonding position to R³ in formula (AN1).

*^{b} represents a bonding position to —C(R¹)(R²)— in formula (AN1).

X and Y each independently represent an integer of from 0 to 10 and preferably an integer of 0 to 3.

R^{2a} and R^{2b} each independently represent a hydrogen atom or a substituent.

When a plurality of R^{2a}'s are present, they may be the same or different. When a plurality of R^{2b}'s are present, they may be the same or different.

When Y is 1 or more, R^{2b} in CR^{2b}₂ that is bonded directly to —C(R¹)(R²)— in formula (AN1) differs from a fluorine atom.

Q represents *^{A}—O—CO—O—*^{B}, *^{A}—CO—*^{B}, *^{A}—CO—O—*^{B}, *^{A}—O—CO—*^{B}, *^{A}—O—*^{B}, *^{A}—S—*^{B}, or When X+Y in formula (AN1-1) is 1 or more and R^{2a}'s and R^{2b}'s in formula (AN1-1) are each a hydrogen atom, and Q represents *^{A}-O—CO—O—*^{B}, *^{A}-CO—*^{B}, *^{A}-O—CO—*^{B}, *^{A}-O—*^{B}, *^{A}—S—*^{B}, or

*^{A} represents a bonding position on the R³ side in formula (AN1), and *^{B} represents a bonding position on the —SO₃⁻ side in formula (AN1).

In formula (AN1), R³ represents an organic group.

No particular limitation is imposed on the organic group, so long as it has at least one carbon atom. The organic group may be a linear group (e.g., a linear alkyl group), a branched group (e.g., a branched alkyl group such as a t-butyl group), or a cyclic group. The organic group may or may not have a substituent. The organic group may or may not have a heteroatom (such as an oxygen atom, a sulfur atom, and/or a nitrogen atom).

In particular, R³ is preferably an organic group having a ring structure. The ring structure may be a monocyclic structure or a polycyclic structure and may have a substituent. Preferably, the ring in the organic group including the ring structure is bonded directly to L in formula (AN1).

The organic group having the ring structure may or may not have, for example, a heteroatom (for example, an oxygen atom, a sulfur atom, and/or, a nitrogen atom). At least one carbon atom included in the ring structure may be replaced with a heteroatom.

The organic group having the ring structure is preferably a hydrocarbon group having a ring structure, a lactone ring group, or a sultone ring group. In particular, the organic group having the ring structure is preferably a hydrocarbon group having a ring structure.

The hydrocarbon group having a ring structure is preferably a monocyclic or polycyclic cycloalkyl group. These groups may have a substituent.

The cycloalkyl group may be a monocyclic group (such as a cyclohexyl group) or a polycyclic group (such as an adamantyl group), and the number of carbon atoms is preferably 5 to 12.

Preferably, the lactone group and the sultone group are each, for example, a group formed by removing one hydrogen atom from a ring member atom included in the lactone or sultone structure in any of the structures represented by formulas (LC1-1) to (LC1-21) and formulas (SL1-1) to (SL1-3) described above.

The non-nucleophilic anion may be a benzenesulfonate anion and is preferably a benzenesulfonate anion substituted with a branched alkyl group or a cycloalkyl group.

The non-nucleophilic anion is also preferably an anion represented by the following formula (AN2).

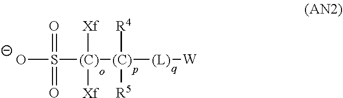

In formula (AN2), o represents an integer of from 1 to 3. p represents an integer of from 0 to 10. q represents an integer of from 0 to 10.

Xf's each represent a hydrogen atom, a fluorine atom, an alkyl group substituted with at least one fluorine atom, or an organic group having no fluorine atom. The number of carbon atoms in the alkyl group is preferably 1 to 10 and more preferably 1 to 4. The alkyl group substituted with at least one fluorine atom is preferably a perfluoroalkyl group.

Xf's are each preferably a fluorine atom or a perfluoroalkyl group having 1 to 4 carbon atoms and more preferably a fluorine atom or CF₃. It is still more preferable that each of Xf's is a fluorine atom.

R⁴ and R⁵ each independently represent a hydrogen atom, a fluorine atom, an alkyl group, or an alkyl group substituted with at least one fluorine atom. When a plurality of R⁴'s are present, they may be the same or different. When a plurality of R⁵'s are present, they may be the same or different.

The number of carbon atoms in each of the alkyl groups represented by R⁴ and R⁵ is preferably 1 to 4. Each alkyl group may have a substituent. R₄ and R₅ are each preferably a hydrogen atom.

L represents a divalent linking group. The definition of Lis the same as the definition of L in formula (AN1).

W represents an organic group including a ring structure. In particular, W is preferably a cyclic organic group.

Examples of the cyclic organic group include alicyclic groups, aryl groups, and heterocyclic groups.

The alicyclic group may be a monocyclic alicyclic group or a polycyclic alicyclic group. Examples of the monocyclic alicyclic group include monocyclic cycloalkyl groups such as a cyclopentyl group, a cyclohexyl group, and a cyclooctyl group. Examples of the polycyclic alicyclic group include polycyclic cycloalkyl groups such as a norbornyl group, a tricyclodecanyl group, a tetracyclodecanyl group, a tetracyclododecanyl group, and an adamantyl group. Of these, alicyclic groups having 7 or more carbon atoms and a bulky structure such as a norbornyl group, a tricyclodecanyl group, a tetracyclodecanyl group, a tetracyclododecanyl group, and an adamantyl group are preferred.

The aryl group may be a monocyclic or polycyclic aryl group. Examples of such an aryl group include a phenyl group, a naphthyl group, a phenanthryl group, and an anthryl group.

The heterocyclic group may be a monocyclic or polycyclic heterocyclic group. In particular, when the heterocyclic group is a polycyclic heterocyclic group, diffusion of acid can be further reduced. The heterocyclic group may or may not have aromaticity. Examples of the heterocycle having aromaticity include a furan ring, a thiophene ring, a benzofuran ring, a benzothiophene ring, a dibenzofuran ring, a dibenzothiophene ring, and a pyridine ring. Examples of the heterocycle having no aromaticity include a tetrahydropyran ring, lactone rings, sultone rings, and a decahydroisoquinoline ring. The heterocycle in the heterocyclic group is preferably a furan ring, a thiophene ring, a pyridine ring, or a decahydroisoquinoline ring.

The above cyclic organic group may have a substituent. Examples of the substituent include alkyl groups (which may be linear or branched and have preferably 1 to 12 carbon atoms), cycloalkyl groups (which may be monocyclic, polycyclic, or spirocyclic and have preferably 3 to 20 carbon atoms), aryl groups (having preferably 6 to 14 carbon atoms), a hydroxy group, alkoxy groups, ester groups, amido groups, urethane groups, ureide groups, thioether groups, sulfonamido groups, and sulfonate groups. Carbon included in the cyclic organic group (carbon contributing to the formation of the ring) may be carbonyl carbon.

The anion represented by formula (AN2) is preferably SO₃⁻—CF₂—CH₂—OCO-(L)_{q′}-W, SO₃⁻—CF₂—CHF—CH₂—OCO-(L)_{q′}-W, SO₃⁻—CF₂—COO-(L)_{q′}-W, SO₃⁻—CF₂—CF₂—CH₂—CH₂-(L)_{q}-W, or SO₃⁻—CF₂—CH(CF₃)—OCO-(L)_{q′}-W. L, q, and W are the same as those in formula (AN2). q′ represents an integer of from 0 to 10.

The non-nucleophilic anion is also preferably an aromatic sulfonate anion represented by the following formula (AN3).

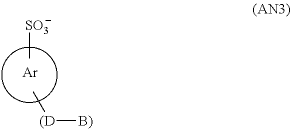

In formula (AN3), Ar represents an aryl group (such as a phenyl group) and may further have a substituent other than a sulfonate anion and the -(D-B) group. Examples of the substituent include a fluorine atom and a hydroxy group.

n represents an integer of 0 or more. n is preferably 1 to 4, more preferably 2 to 3, and still more preferably 3.

D represents a single bond or a divalent linking group. Examples of the divalent linking group include ether groups, thioether groups, a carbonyl group, sulfoxide groups, a sulfone group, sulfonate groups, ester groups, and groups formed by combining two or more of these groups.

B represents a hydrocarbon group.

B is preferably an aliphatic hydrocarbon group and more preferably an isopropyl group, a cyclohexyl group, or an aryl group optionally having a substituent (such as a tricyclohexylphenol group).

The non-nucleophilic anion is also preferably a disulfonamide anion.

The disulfonamide anion is, for example, an anion represented by N⁻(SO₂-R^{q})₂.

Here, R^{q} represents an alkyl group optionally having a substituent and is preferably a fluoroalkyl group and more preferably a perfluoroalkyl group. Two R^{q}'s may be bonded together to form a ring. The group formed by bonding two R^{q}'s is preferably an alkylene group optionally having a substituent, more preferably a fluoroalkylene group, and still more preferably a perfluoroalkylene group. The number of carbon atoms in the alkylene group is preferably 2 to 4.

Other examples of the non-nucleophilic anion include anions represented by the following formulas (d1-1) to (d1-4).

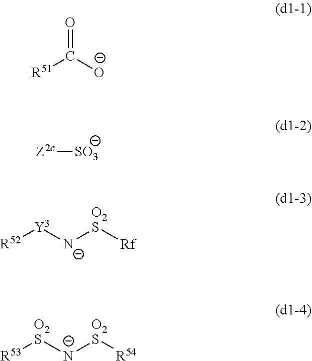

In formula (d1-1), R⁵¹ represents a hydrocarbon group (e.g., an aryl group such as a phenyl group) optionally having a substituent (e.g., a hydroxy group).

In formula (d1-2), Z^{2c} represents a hydrocarbon group having 1 to 30 carbon atoms and optionally having a substituent (provided that the carbon atom adjacent to S is not substituted with a fluorine atom).

The hydrocarbon group represented by Z^{2c} may be linear or branched and may have a ring structure. A carbon atom in the hydrocarbon group (preferably, a carbon atom serving as a ring member atom when the hydrocarbon group has a ring structure) may be carbonyl carbon (CO—). Examples of the hydrocarbon group include a group having a norbornyl group optionally having a substituent. A carbon atom included in the norbornyl group may be carbonyl carbon.

It is preferable that “Z²—SO₃⁻” in formula (d1-2) differs from the anions represented by formulas (AN1) to (AN3) above. For example, Z^{2c} differs from an aryl group. For example, in Z², the atoms at the α- and β-positions with respect to —SO₃⁻ are each preferably an atom different from a carbon atom having a fluorine atom as a substituent. For example, in Z^{2c}, the atom at the α-position and/or the atom at the 3-position with respect to —SO₃⁻ is preferably a ring member atom of the cyclic group.

In formula (d1-3), R⁵² represents an organic group (preferably a hydrocarbon group having a fluorine atom), and Y³ represents a linear, branched, or cyclic alkylene group, an arylene group, or a carbonyl group. Rf represents a hydrocarbon group.

In formula (d1-4), R⁵³ and R⁵⁴ each independently represent an organic group (preferably a hydrocarbon group having a fluorine atom). R⁵³ and R⁵⁴ may be bonded together to form a ring.

One type of organic anion may be used alone, or two or more types of organic anions may be used.

It is also preferable that the photoacid generator is at least one selected from the group consisting of compounds (1) to (2).

### (Compound (1))

The compound (1) is a compound having at least one structural moiety X described below and at least one structural moiety Y described below and is a compound that generates an acid including a first acidic moiety described below and derived from the structural moiety X and a second acidic moiety described below and derived from the structural moiety Y when irradiated with actinic rays or radiation.

Structural moiety X: A structural moiety including an anionic moiety A₁⁻ and a cationic moiety M₁⁺ and forms the first acidic moiety represented by HA₁ when irradiated with actinic rays or radiation.

Structural moiety Y: A structural moiety including an anionic moiety A₂⁻ and a cationic moiety M₂⁺ and forms the second acidic moiety represented by HA₂ when irradiated with actinic rays or radiation.

The compound (1) satisfies the following condition I.

Condition I: A compound PI formed by replacing each of the cationic moiety M₁⁺ in the structural moiety X in the compound (1) and the cationic moiety M₂ in the structural moiety Y with H⁺ has an acid dissociation constant a1 derived from the acidic moiety represented by HA₁ formed by replacing the cationic moiety M₁⁺ in the structural moiety X with H⁺ and an acid dissociation constant a2 derived from the acidic moiety represented by HA₂ formed by replacing the cationic moiety M₂ in the structural moiety Y with H⁺, and the acid dissociation constant a2 is larger than the acid dissociation constant a1.

The condition I will be specifically described.

When the compound (1) is a compound that generates an acid having one first acidic moiety derived from the structural moiety X and one second acidic moiety derived from the structural moiety Y, the compound PI corresponds to a “compound having HA₁ and HA₂.”

The acid dissociation constant a1 and the acid dissociation constant a2 of this compound PI will be specifically described. When the acid dissociation constants of the compound PI are determined, the pKa when the compound PI becomes a “compound having A₁⁻ and HA₂” is the acid dissociation constant a1, and the pKa when the “compound having A₁⁻ and HA₂” becomes a “compound having A₁⁻ and A₂” is the acid dissociation constant a2.

When the compound (1) is, for example, a compound that generates an acid having two first acidic moieties derived from the structural moieties X and one second acidic moiety derived from the structural moiety Y, the compound PI corresponds to a “compound having two HA₁'s and one HA₂.”

When the acid dissociation constants of the compound PI are determined, the acid dissociation constant when the compound PI becomes a “compound having one A₁⁻, one HA₁, and one HA₂,” and the acid dissociation constant when the “compound having one A₁⁻, one HA₁, and one HA₂” becomes a “compound having two A₁⁻'s and one HA₂” each correspond to the acid dissociation constant a1. Moreover, the acid dissociation constant when the “compound having two A₁⁻'s and one HA₂” becomes a “compound having two A₁⁻'s and A₂⁻” corresponds to the acid dissociation constant a2. Specifically, such a compound PI has a plurality of acid dissociation constants derived from acidic moieties represented by HA₁ formed by replacing cationic moieties M₁⁺ in the structural moieties X with H⁺. In this case, the acid dissociation constant a2 is larger than the largest one of the plurality of acid dissociation constants a1. Let aa be the acid dissociation constant when the compound PI becomes the “compound having one A₁⁻, one HA₁, and one HA₂,” and ab be the acid dissociation constant when the “compound having one A₁⁻, one HA₁, and one HA₂” becomes the “compound having two A₁⁻'s and one HA₂.” Then aa and ab satisfy the relation aa<ab.

The acid dissociation constants a1 and a2 are determined by the acid dissociation constant measurement method described above.

The compound PI corresponds to the acid generated when the compound (1) is irradiated with actinic rays or radiation.

When the compound (1) has two or more structural moieties X, these structural moieties X may be the same or different. Moreover, two or more A₁⁻'s may be the same or different, and two or more M₁⁺'s may be the same or different.

In compound (1), A₁⁻ and A₂⁻ may be the same or different, and M₁⁺ and M₂ may be the same or different. However, it is preferable that A₁⁻ and A₂ are different from each other.

In the compound PI, the difference (absolute difference) between the acid dissociation constant a1 (when a plurality of acid dissociation constants a1 are present, the maximum value of the acid dissociation constants) and the acid dissociation constant a2 is preferably 0.1 or more, more preferably 0.5 or more, and still more preferably 1.0 or more. No particular limitation is imposed on the upper limit of the difference (absolute difference) between the acid dissociation constant a1 (when a plurality of acid dissociation constants a1 are present, the maximum value of the acid dissociation constants) and the acid dissociation constant a2, but the upper limit is, for example, 16 or less.

In the compound PI, the acid dissociation constant a2 is preferably 20 or less and more preferably 15 or less. The lower limit of the acid dissociation constant a2 is preferably −4.0 or more.

In the compound PI, the acid dissociation constant a1 is preferably 2.0 or less and more preferably 0 or less. The lower limit of the acid dissociation constant a1 is preferably −20.0 or more.

The anionic moiety A₁⁻ and the anionic moiety A₂ are each a structural moiety including a negatively charged atom or atomic group and are each, for example, a structural moiety selected from the group consisting of formulas (AA-1) to (AA-3) and formulas (BB-1) to (BB-6) shown below.

The anionic moiety A₁⁻ is preferably a moiety capable of forming an acidic moiety having a small acid dissociation constant, more preferably a moiety represented by any one of (AA-1) to (AA-3), and still more preferably a moiety represented by any one of formulas (AA-1) and (AA-3).

The anionic moiety A₂ is preferably a moiety capable of forming an acidic moiety having a larger acid dissociation constant than the anionic moiety A₁⁻, more preferably a moiety represented by any one of formulas (BB-1) to (BB-6), and still more preferably a moiety represented by any one of formulas (BB-1) and (BB-4).

In formulas (AA-1) to (AA-3) and formulas (BB-1) to (BB-6) below, * represents a bonding position.

In formula (AA-2), R^{A} represents a monovalent organic group. No particular limitation is imposed on the monovalent organic group represented by R^{A}, and examples thereof include a cyano group, a trifluoromethyl group, and a methanesulfonyl group.

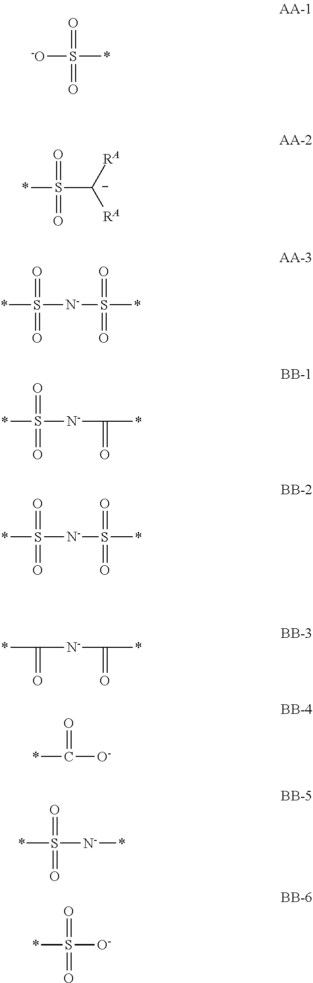

The cationic moiety M₁⁺ and the cationic moiety M₂ are each a structural moiety including a positively charged atom or atomic group and are each, for example, a singly charged organic cation. Examples of the organic cation include the above-described organic cation represented by M⁺.

No particular limitation is imposed on the specific structure of the compound (1), and examples thereof include compounds represented by formulas (Ia-1) to (Ia-5) described below.

### —Compound Represented by Formula (Ia-1)—

First, the compound represented by formula (Ia-1) will be described.

M₁₁⁺A₁₁⁻-L₁-A₁₂⁻M₁₂ (Ia-1)

The compound represented by formula (Ia-1) generates an acid represented by HA₁₁-L₁-A₁₂H when irradiated with actinic rays or radiation.

In formula (Ia-1), M₁₁⁺ and M₁₂ each independently represent an organic cation.
A₁₁⁻ and Ala each independently represent a monovalent anionic functional group.
L₁ represents a divalent linking group.
M₁₁⁺ and M₁₂ may be the same or different.
A₁₁⁻ and Ala may be the same or different, but it is preferable that they differ from each other.

In a compound PIa (HA₁₁-L₁-A₁₂H) formed by replacing each of the cations represented by M₁₁⁺ and M₁₂ in formula (Ia-1) above with H⁺, the acid dissociation constant a2 derived from the acidic moiety represented by A₁₂H is larger than the acid dissociation constant a1 derived from the acidic moiety represented by HA₁₁. Preferred values of the acid dissociation constants a1 and a2 are as described above. The compound PIa is the same as the acid generated from the compound represented by formula (Ia-1) upon irradiation with actinic rays or radiation.

At least one of M₁₁⁺, M₁₂⁺, A₁₁⁻, A₁₂, or L₁ may have an acid-decomposable group as a substituent.

Examples of the organic cations represented by M₁₁⁺ and M₁₂⁺ in formula (Ia-1) include those for the organic cation represented by M⁺ above.

The monovalent anionic functional group represented by A₁₁⁻ means a monovalent group including the anionic moiety A₁⁻ described above. The monovalent anionic functional group represented by A₁₂ means a monovalent group including the anionic moiety A₂ described above.

The monovalent anionic functional groups represented by A₁₁⁻ and A₁₂⁻ are each preferably a monovalent anionic functional group including the anionic moiety represented by any of formulas (AA-1) to (AA-3) and formulas (BB-1) to (BB-6) described above and more preferably a monovalent anionic functional group selected from the group consisting of formulas (AX-1) to (AX-3) and formulas (BX-1) to (BX-7). In particular, the monovalent anionic functional group represented by A₁₁⁻ is preferably a monovalent anionic functional group represented by any of formulas (AX-1) to (AX-3). In particular, the monovalent anionic functional group represented by A₁₂ is preferably a monovalent anionic functional group represented by any of formulas (BX-1) to (BX-7) and more preferably a monovalent anionic functional group represented by any of formulas (BX-1) to (BX-6).

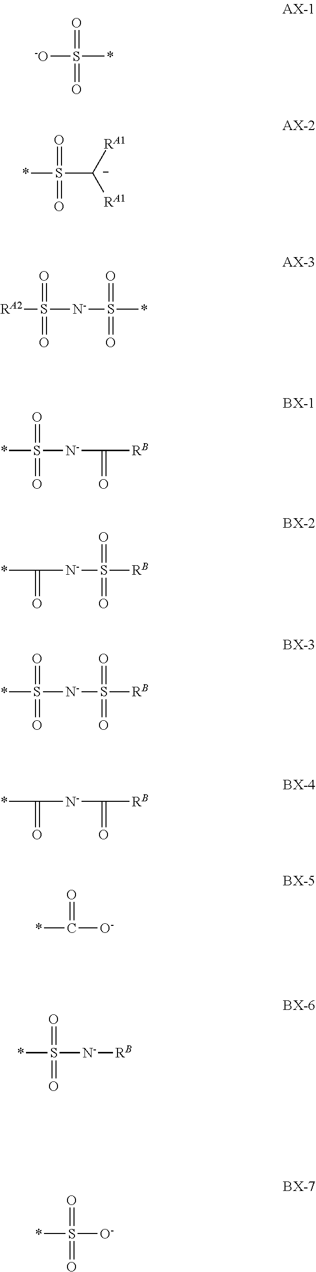

In formulas (AX-1) to (AX-3), RAI and R¹ each independently represent a monovalent organic group. * represents a bonding position.

No particular limitation is imposed on the monovalent organic group represented by R^{A1} and examples thereof include a cyano group, a trifluoromethyl group, and a methanesulfonyl group.

The monovalent organic group represented by R is preferably a linear, branched, or cyclic alkyl group or an aryl group.

The number of carbon atoms in the alkyl group is preferably 1 to 15, more preferably 1 to 10, and still more preferably 1 to 6.

The alkyl group may have a substituent. The substituent is preferably a fluorine atom or a cyano group and more preferably a fluorine atom. When the alkyl group has a fluorine atom as a substituent, the alkyl group may be a perfluoroalkyl group.

The aryl group is preferably a phenyl group or a naphthyl group and more preferably a phenyl group.

The aryl group may have a substituent. The substituent is preferably a fluorine atom, an iodine atom, a perfluoroalkyl group (for example, having preferably 1 to 10 carbon atoms and more preferably 1 to 6 carbon atoms), or a cyano group and is more preferably a fluorine atom, an iodine atom, or a perfluoroalkyl group.

In formulas (BX-1) to (BX-4) and (BX-6), RB represents a monovalent organic group. represents a bonding position.

The monovalent organic group represented by RB is preferably a linear, branched, or cyclic alkyl group or an aryl group.

The number of carbon atoms in the alkyl group is preferably 1 to 15, more preferably 1 to 10, and still more preferably 1 to 6.

The alkyl group may have a substituent. No particular limitation is imposed on the substituent, but the substituent is preferably a fluorine atom or a cyano group and more preferably a fluorine atom. When the alkyl group has a fluorine atom as a substituent, the alkyl group may be a perfluoroalkyl group.

When a carbon atom in the alkyl group at a bonding position (for example, the carbon atom in the alkyl group that is bonded directly to —CO— in any of formulas (BX-1) and (BX-4), the carbon atom shown in the alkyl group that is bonded directly to —SO₂— in any of formulas (BX-2) and (BX-3), or the carbon atom shown in the alkyl group that is bonded directly to N⁻ in formula (BX-6)) has a substituent, it is also preferable that the substituent differs from a fluorine atom and a cyano group.

In the alkyl group, a carbon atom may be replaced with carbonyl carbon.

The aryl group is preferably a phenyl group or a naphthyl group and more preferably a phenyl group.

The aryl group may have a substituent. The substituent is preferably a fluorine atom, an iodine atom, a perfluoroalkyl group (for example, having preferably 1 to 10 carbon atoms and more preferably 1 to 6 carbon atoms), a cyano group, an alkyl group (for example, having preferably 1 to 10 carbon atoms and more preferably 1 to 6 carbon atoms), an alkoxy group (for example, having preferably 1 to 10 carbon atoms and more preferably 1 to 6 carbon atoms), or an alkoxycarbonyl group (for example, having preferably 2 to 10 carbon atoms and more preferably 2 to 6 carbon atoms) and more preferably a fluorine atom, an iodine atom, a perfluoroalkyl group, an alkyl group, an alkoxy group, or an alkoxycarbonyl group.

In formula (Ia-1), no particular limitation is imposed on the divalent linking group represented by L₁, and examples of L₁ include —CO—, —NR—, —O—, —S—, —SO—, —SO₂—, alkylene groups (which may be linear or branched and have preferably 1 to 6 carbon atoms), cycloalkylene groups (having preferably 3 to 15 carbon atoms), alkenylene groups (having preferably 2 to 6 carbon atoms), divalent aliphatic heterocyclic groups (preferably 5- to 10-membered rings, more preferably 5- to 7-membered rings, and still more preferably 5- to 6-membered rings, each of which has at least one N atom, 0 atom, S atom, or Se atom in the ring structure), divalent aromatic heterocyclic groups (preferably 5- to 10-membered rings, more preferably 5- to 7-membered rings, and still more preferably 5- to 6-membered rings, each of which has at least one N atom, 0 atom, S atom, or Se atom in the ring structure), divalent aromatic hydrocarbon ring groups (preferably 6- to 10-membered rings and more preferably 6-membered rings), and divalent linking groups formed by combining a plurality of groups selected from the above groups. R is a hydrogen atom or a monovalent organic group. No particular limitation is imposed on the monovalent organic group, but the monovalent organic group is preferably, for example, an alkyl group (having preferably 1 to 6 carbon atoms).

The alkylene, cycloalkylene, alkenylene, divalent aliphatic heterocyclic, divalent aromatic heterocyclic, and divalent aromatic hydrocarbon ring groups described above may each have a substituent. Examples of the substituent include halogen atoms (preferably a fluorine atom).

In particular, the divalent linking group represented by L₁ is preferably a divalent linking group represented by formula (L₁).

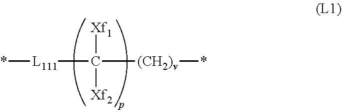

In formula (L1), L₁₁₁ represents a single bond or a divalent linking group.

No particular limitation is imposed on the divalent linking group represented by L₁₁₁, and examples of the divalent linking group include —CO—, —NH—, —O—, —SO—, —SO₂—, alkylene groups (which may be linear or branched and have preferably 1 to 6 carbon atoms) optionally having a substituent, cycloalkylene groups (having preferably 3 to 15 carbon atoms) optionally having a substituent, aryl groups (having preferably 6 to 10 carbon atoms) optionally having a substituent, and divalent linking groups formed by combining a plurality of groups selected from the above groups. No particular limitation is imposed on the substituent, and examples thereof include halogen atoms.

p represents an integer of from 0 to 3 and preferably represents an integer of from 1 to 3.

v represents an integer of 0 or 1.

Xf₁'s each independently represent a fluorine atom or an alkyl group substituted with at least one fluorine atom. The number of carbon atoms in the alkyl group is preferably 1 to 10 and more preferably 1 to 4. The alkyl group substituted with at least one fluorine atom is preferably a perfluoroalkyl group.

Xf₂ s each independently represent a hydrogen atom, an alkyl group optionally having a fluorine atom as a substituent, or a fluorine atom. The number of carbon atoms in the alkyl group is preferably 1 to 10 and more preferably 1 to 4. In particular, each Xf₂ represents preferably a fluorine atom or an alkyl group substituted with at least one fluorine atom and more preferably a fluorine atom or a perfluoroalkyl group.

In particular, Xf₁'s and Xf₂ s each independently represent preferably a fluorine atom or a perfluoroalkyl group having 1 to 4 carbon atoms and more preferably a fluorine atom or CF₃. In particular, it is still more preferable that Xf₁'s and Xf₂'s are each a fluorine atom.

* represents a bonding position.

When L₁ in formula (Ia-1) represents a divalent linking group represented by formula (L₁), it is preferable that the direct bond (*) on the L₁₁₁ side in formula (L₁) is bonded to A₁₂ in formula (Ia-1).

### —Compounds Represented be Formulas (Ia-2) to (Ia-4)—

Next, compounds represented by formulas (Ia-2) to (Ia-4) will be described.

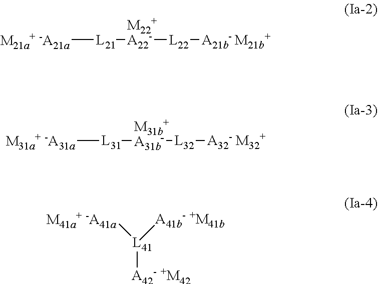

In formula (Ia-2), A₂₁ₐ⁻ and A_{21b}⁻ each independently represent a monovalent anionic functional group. Each of the monovalent anionic functional groups represented by A₂₁ₐ⁻ and A_{21b}⁻ means a monovalent group including the above-described anionic moiety A₁⁻. No particular limitation is imposed on the monovalent anionic functional groups represented by A₂₁ₐ⁻ and A_{21b}⁻, but A₂₁ₐ⁻ and A_{21b}⁻ are each, for example, a monovalent anionic functional group selected from the group consisting of formulas (AX-1) to (AX-3) described above.

A₂₂⁻ represents a divalent anionic functional group. The divalent anionic functional group represented by A₂₂⁻ means a divalent groups including the anionic moiety A₂⁻ described above. Examples of the divalent anionic functional group represented by A₂₂⁻ include divalent anionic functional groups represented by the following formulas (BX-8) to (BX-11).

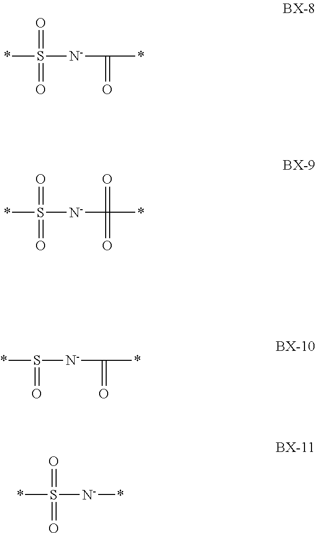

M₂₁ₐ⁺, M_{21b}⁺, and M₂₂ each independently represent an organic cation. The definitions of the organic cations represented by M₂₁ₐ⁺, M_{21b}⁺, and M₂₂ are the same as that of M₁₁⁺ described above, and their preferred modes are also the same as those of M₁₁⁺.

L₂₁ and L₂₂ each independently represent a divalent organic group.

In a compound PIa-2 formed by replacing each of the organic cations represented by M₂₁ₐ⁺, M_{21b}⁺, and M₂₂⁺ in formula (Ia-2) with H⁺, the acid dissociation constant a2 derived from an acidic moiety represented by A₂₂H is larger than the acid dissociation constant a1-1 derived from an acidic moiety represented by A₂₁ₐH and the acid dissociation constant a1-2 derived from an acidic moiety represented by A_{21b}H. The acid dissociation constant a1-1 and the acid dissociation constant a1-2 each correspond to the acid dissociation constant a1 described above.

A₂₁ₐ⁻ and A_{21b}⁻ may be the same or different. M₂₁ₐ⁺, M_{21b}⁺, and M₂₂⁺ may be the same or different.

At least one of M₂₁ₐ⁺, M_{21b}⁺, M₂₂⁺, A₂₁ₐ⁺, A_{21b}⁻, L₂₁, or L₂₂ may have an acid-decomposable group as a substituent.

In formula (Ia-3), A₃₁ₐ⁻ and A₃₂⁻ each independently represent a monovalent anionic functional group. The definition of the monovalent anionic functional group represented by A₃₁ₐ⁻ is the same as those of A₂₁ₐ⁻ and A_{21b}⁻ in formula (Ia-2), and its preferred mode is also the same as those of A₂₁ₐ⁻ and A_{21b}⁻.

The monovalent anionic functional group represented by A₃₂ means a monovalent group including the anionic moiety A₂ described above. No particular limitation is imposed on the monovalent anionic functional group represented by A₃₂⁻, and A₃₂⁻ is, for example, a monovalent anionic functional group selected from the group consisting of formulas (BX-1) to (BX-7) described above.

A_{31b}⁻ represents a divalent anionic functional group. The divalent anionic functional group represented by A_{31b}⁻ means a divalent group including the anionic moiety A₁⁻ described above. Examples of the divalent anionic functional group represented by A_{31b}⁻ include a divalent anionic functional group represented by the following formula (AX-4).

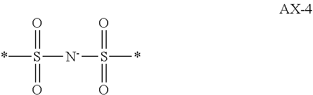 [SMILES: *S(=O)(=O)[N-]S(*)(=O)=O]

M₃₁ₐ⁺, M_{31b}⁺, and M₃₂⁺ each independently represent a monovalent organic cation. The definitions of the organic cations represented by M₃₁ₐ⁺, M_{31b}⁺, and M₃₂⁺ are the same are that of M₁⁺ described above, and their preferred modes are also the same as that of M₁₁⁺.

L₃₁ and L₃₂ each independently represent a divalent organic group.

In a compound PIa-3 formed by replacing each of the organic cations represented by M₃₁ₐ⁺, M_{31b}⁺, and M₃₂⁺ in formula (Ia-3) with H⁺, the acid dissociation constant a2 derived from an acidic moiety represented by A₃₂H is larger than the acid dissociation constant a1-3 derived from an acidic moiety represented by A₃₁ₐH and the acid dissociation constant a1-4 derived from an acidic moiety represented by A_{31b}H. The acid dissociation constant a1-3 and the acid dissociation constant a1-4 each correspond to the acid dissociation constant a1 described above.

A₃₁ₐ⁻ and A₃₂⁻ may be the same or different. M₃₁ₐ⁺, M_{31b}⁺, and M₃₂ may be the same or different.

At least one of M₃₁ₐ⁺, M_{31b}⁺, M₃₂⁺, A₃₁ₐ⁻, A₃₂⁻, L₃₁, or L₃₂ may have an acid-decomposable group as a substituent.

In formula (Ia-4), A₄₁ₐ⁻, A_{41b}⁻, and A₄₂⁻ each independently represent a monovalent anionic functional group. The definitions of the monovalent anionic functional groups represented by A₄₁ₐ⁻ and A_{41b}⁻ are the same as the definitions of A₂₁ₐ⁻ and A_{21b}⁻ in formula (Ia-2) described above. The definition of the monovalent anionic functional group represented by A₄₂⁻ is the same as that of A₃₂⁻ in formula (Ia-3) described above, and its preferred mode is also the same as that of A₃₂.

M₄₁ₐ⁺, M_{41b}⁺, and M₄₂ each independently represent an organic cation. The organic cations represented by M₄₁ₐ⁺, M_{41b}⁺, and M₄₂ are the same as that represented by M₁₁⁺ described above, and their preferred modes are also the same as that of M₁₁⁺.

L₄₁ represents a trivalent organic group.

In a compound PIa-4 formed by replacing each of the organic cations represented by M₄₁ₐ⁺, M_{41b}⁺, and M₄₂ in formula (Ia-4) with H⁺, the acid dissociation constant a2 derived from an acidic moiety represented by A₄₂H is larger than the acid dissociation constant a1-5 derived from an acidic moiety represented by A₄₁ₐH and the acid dissociation constant a1-6 derived from an acidic moiety represented by A_{41b}H. The acid dissociation constant a1-5 and the acid dissociation constant a1-6 each correspond to the acid dissociation constant a1 described above.

A₄₁ₐ⁻, A_{41b}⁻, and A₄₂⁻ may be the same or different. M₄₁ₐ⁺, M_{41b}⁺, and M₄₂⁺ may be the same or different.

At least one of M₄₁ₐ⁺, M_{41b}⁺, M₄₂⁺, A₄₁ₐ⁻, A_{41b}⁻, A₄₂, or L₄₁ may have an acid-decomposable group as a substituent.

No particular limitation is imposed on the divalent organic groups represented by L₂₁ and L₂₂ in In formula (Ia-2) and L₃₁ and L₃₂ in formula (Ia-3), and examples thereof include —CO—, —NR—, —O—, —S—, —SO—, —SO₂—, alkylene groups (which may be linear or branched and have preferably 1 to 6 carbon atoms), cycloalkylene groups (having preferably 3 to 15 carbon atoms), alkenylene groups (having preferably 2 to 6 carbon atoms), divalent aliphatic heterocyclic groups (preferably 5- to 10-membered rings, more preferably 5- to 7-membered rings, and still more preferably 5- to 6-membered rings, each of which has at least one N atom, 0 atom, S atom, or Se atom in the ring structure), divalent aromatic heterocyclic groups (preferably 5- to 10-membered rings, more preferably 5- to 7-membered rings, and still more preferably 5- to 6-membered rings, each of which has at least one N atom, 0 atom, S atom, or Se atom in the ring structure), divalent aromatic hydrocarbon ring groups (preferably 6- to 10-membered rings and more preferably 6-membered rings), and divalent organic groups formed by combining a plurality of groups selected from the above groups. Examples of R include a hydrogen atom and monovalent organic groups. No particular limitation is imposed on the monovalent organic group, but the monovalent organic group is, for example, preferably an alkyl group (having preferably 1 to 6 carbon atoms).

The alkylene, cycloalkylene, alkenylene, divalent aliphatic heterocyclic, divalent aromatic heterocyclic, and divalent aromatic hydrocarbon ring groups described above may each have a substituent. Examples of the substituent include halogen atoms (preferably a fluorine atom).

Preferably, the divalent organic groups represented by L₂₁ and L₂₂ in formula (Ia-2) and Lai and L₃₂ in formula (Ia-3) are each, for example, a divalent organic group represented by formula (L₂) below.

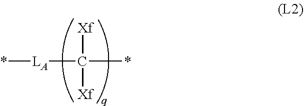

In formula (L₂), q represents an integer of from 1 to 3. * represents a bonding position.

Xf's each independently represent a fluorine atom or an alkyl group substituted with at least one fluorine atom. The number of carbon atoms in the alkyl group is preferably 1 to 10 and more preferably 1 to 4. The alkyl group substituted with at least one fluorine atom is preferably a perfluoroalkyl group.

Xf's are each preferably a fluorine atom or a perfluoroalkyl group having 1 to 4 carbon atoms and more preferably a fluorine atom or CF₃. In particular, it is more preferable that both Xf's are fluorine atoms.

L_{A} represents a single bond or a divalent linking group.

No particular limitation is imposed on the divalent linking group represented by L_{A}, and examples thereof include —CO—, —O—, —SO—, —SO₂—, alkylene groups (which may be linear or branched and have preferably 1 to 6 carbon atoms), cycloalkylene groups (having preferably 3 to 15 carbon atoms), divalent aromatic hydrocarbon ring groups (preferably 6- to 10-membered rings and more preferably 6-membered rings), and divalent linking groups formed by combining a plurality of groups selected from the above groups.

The alkylene, cycloalkylene, and divalent aromatic hydrocarbon ring groups described above may each have a substituent. Examples of the substituent include halogen atoms (preferably a fluorine atom).

Examples of the divalent organic group represented by formula (L₂) include *—CF₂—*, *—CF₂—CF₂—*, *—CF₂—CF₂—CF₂—*, *-Ph-O—SO₂—CF₂—*, *-Ph-O—SO₂—CF₂—CF₂—*, *-Ph-O—SO₂—CF₂—CF₂—CF₂—*, and *-Ph-OCO—CF₂—*. Ph is a phenylene group optionally having a substituent and is preferably a 1,4-phenylene group. No particular limitation is imposed on the substituent, but the substituent is preferably an alkyl group (for example, having preferably 1 to 10 carbon atoms and more preferably 1 to 6 carbon atoms), an alkoxy group (for example, having preferably 1 to 10 carbon atoms and more preferably 1 to 6 carbon atoms), or an alkoxycarbonyl group (for example, having preferably 2 to 10 carbon atoms and more preferably 2 to 6 carbon atoms).

When L₂₁ and L₂₂ in formula (Ia-2) each represent the divalent organic group represented by formula (L2), it is preferable that the direct bond (*) on the L_{A} side in formula (L2) is bonded to A₂₁a or A_{21b}⁻ in formula (Ia-2).

When L₃₁ and L₃₂ in formula (Ia-3) each represent the divalent organic group represented by formula (L2), it is preferable that the direct bond (*) on the L_{A} side in formula (L2) is bonded to A₃₁a or A₃₂ in formula (Ia-3).

### —Compound Represented by Formula (Ia-5)—

Next, formula (Ia-5) will be described.

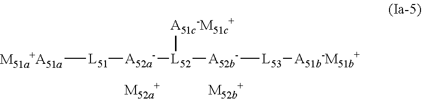

In formula (Ia-5), A₅₁ₐ⁻, A_{51b}⁻, and A_{51c}⁻ each independently represent a monovalent anionic functional group. Each of the monovalent anionic functional groups represented by A₅₁ₐ⁻, A_{51b}⁻, and A_{51c}⁻ means a monovalent group including the anionic moiety A₁⁻ described above. No particular limitation is imposed on the monovalent anionic functional groups represented by A₅₁ₐ⁻, A_{51b}⁻, and A_{51c}⁻, but each of these monovalent anionic functional groups is, for example, a monovalent anionic functional group selected from the group consisting of formulas (AX-1) to (AX-3) described above.

A₅₂ₐ⁻ and A_{52b}⁻ each represent a divalent anionic functional group. Each of the divalent anionic functional groups represented by A₅₂ₐ⁻ and A_{52b}⁻ means a divalent group including the anionic moiety A₂⁻ described above. The divalent anionic functional groups represented by A₅₂ₐ⁻ and A_{52b}⁻ are each, for example, a divalent anionic functional groups selected from the group consisting of formulas (BX-8) to (BX-11) described above.

M₅₁ₐ⁺, M_{51b}⁺, M_{51c}⁺, M₅₂ₐ⁺, and M_{52b}⁺ each independently represent an organic cation. The definitions of the organic cations represented by M₅₁ₐ⁺, M_{51b}⁺, M_{51c}⁺, M₅₂ₐ⁺, and M_{52b}⁺ are the same as the definition of M₁₁⁺ described above, and their preferred modes are also the same as that of M₁⁺.

L₅₁ and L₅₃ each independently represent a divalent organic group. The definitions of the divalent organic groups represented by L₅₁ and L₅₃ are the same as those of L₂₁ and L₂₂ in formula (Ia-2) described above, and their preferred modes are also the same as those of L₂₁ and L₂₂.

L₅₂ represents a trivalent organic group. The definition of the trivalent organic group represented by L₅₂ is the same as that of L₄₁ in formula (Ia-4) described above, and its preferred mode is also the same as that of L₄₁.

In a compound PIa-5 formed by replacing each of the organic cations represented by M₅₁ₐ⁺, M_{51b}⁺, M_{51c}⁺, M₅₂ₐ⁺, and M_{52b}⁺ in formula (Ia-5) with H⁺, the acid dissociation constant a2-1 derived from an acidic moiety represented by A₅₂ₐH and the acid dissociation constant a2-2 derived from an acidic moiety represented by A_{52b}H are larger than the acid dissociation constant a1-1 derived from an acidic moiety represented by A₅₁ₐH, the acid dissociation constant a1-2 derived from an acidic moiety represented by A_{51b}H, and the acid dissociation constant a1-3 derived from an acidic moiety represented by A_{51c}H. The acid dissociation constants a1-1 to a1-3 each correspond to the acid dissociation constant a1 described above, and the acid dissociation constants a2-1 and a2-2 each correspond to the acid dissociation constant a2 described above.

A₅₁ₐ⁻, A_{51b}⁻, and Asia may be the same or different. A₅₁ₐ⁻ and A_{52b}⁻ may be the same or different. M₅₁ₐ⁺, M_{51b}⁺, M_{51c}⁺, M₅₂ₐ⁺, and M_{52b}⁺ may be the same or different.

At least one of M_{51b}⁺, M_{51c}⁺, M₅₂ₐ⁺, M_{52b}⁺, A₅₁ₐ⁻, A_{51b}⁻, A₅₁, L₅₁, L₅₂, or L₅₃ may have an acid-decomposable group as a substituent.

### (Compound (2))

The compound (2) is a compound that has two or more structural moieties X described above and at least one structural moiety Z described below and is a compound that generates an acid including two or more first acidic moieties derived from the structural moieties X and the structural moiety Z when irradiated with actinic rays or radiation.

Structural moiety Z: Non-ionic moiety capable of neutralizing acid

The definition of the structural moiety X in the compound (2) and the definitions of A₁⁻ and M₁⁺ are the same as that of the structural moiety X in the compound (1) described above and those of A₁⁻ and M₁⁺ in the structural moiety X in the compound (1) described above, and their preferred modes are also the same as those of the compound (1).

In a compound PIT formed by replacing each of the cationic moieties M₁⁺ in the structural moieties X in the compound (2) with H⁺, a preferred range of the acid dissociation constant a1 derived from an acidic moiety represented by HA₁ formed by replacing the cationic moiety M₁⁺ in one of the structural moieties X with H⁺ is the same as that of the acid dissociation constant a1 in the compound PI.

When the compound (2) is, for example, a compound that generates an acid having two first acidic moieties derived from the structural moieties X and the structural moiety Z, the compound PII corresponds to a “compound having two HA₁s.” When the acid dissociation constants of the compound PIT are determined, the acid dissociation constant when the compound PIT becomes a “compound having one A₁⁻ and one HA₁” and the dissociation constant when the “compound having one A₁⁻ and one HA₁” becomes a “compound having two A₁⁻'s” each correspond to the acid dissociation constant a1.

The acid dissociation constant a1 is determined by the acid dissociation constant measurement method described above.

The compound PII corresponds to an acid generated when the compound (2) is irradiated with actinic rays or radiation.

The two or more structural moieties X may be the same or different. The two or more A₁⁻'s may be the same or different, and the two or more M₁⁺'s may be the same or different.

No particular limitation is imposed on the non-ionic moiety that is in the structural moiety Z and capable of neutralizing an acid, and the non-ionic moiety is, for example, preferably a moiety including a functional group having an electron or a group capable of electrostatically interacting with a proton.

Examples of the functional group having an electron or a group capable of electrostatically interacting with a proton include a functional group having a macrocyclic structure such as a cyclic polyether and a functional group having a nitrogen atom having an unshared electron pair not contributing to it-conjugation. The nitrogen atom having an unshared electron pair not contributing to it-conjugation is, for example, a nitrogen atom having a partial structure represented by any of the following formulas.

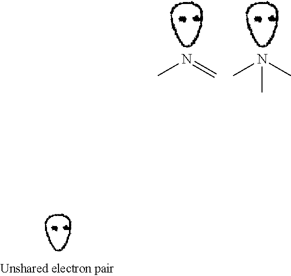

Examples of the partial structure of the functional group having an electron or a group capable of electrostatically interacting with a proton include crown ether structures, azacrown ether structures, primary to tertiary amine structures, a pyridine structure, an imidazole structure, and a pyrazine structure. Of these, primary to tertiary amine structures are preferred.

No particular limitation is imposed on the compound (2), and examples thereof include compounds represented by the following formulas (IIa-1) and (IIa-2).

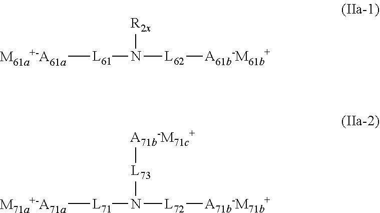

In formula (IIa-1), the definitions of A₆₁ₐ⁻ and A_{61b}⁻ are each the same as that of A₁₁⁻ in formula (Ia-1) above, and their preferred modes are also the same as that of A₁₁⁻. The definitions of M₆₁ₐ⁺ and M_{61b}⁺ are each the same as that of M₁₁⁺ in formula (Ia-1), and their preferred modes are also the same as that of Mill.

In formula (ha-1), the definitions of L₆₁ and L₆₂ are each the same as that of L₁ in formula (Ia-1) above, and their preferred modes are also the same as that of L₁.

In formula (ha-1), R_{2X} represents a monovalent organic group. No particular limitation is imposed on the monovalent organic group represented by R_{2X}, and examples thereof include alkyl groups (which have preferably 1 to 10 carbon atoms and may be linear or branched), cycloalkyl groups (having preferably 3 to 15 carbon atoms), and alkenyl groups (having preferably 2 to 6 carbon atoms). In these groups, —CH₂— may be replaced with one or a combination of two or more selected from the group consisting of —CO—, —NH—, —O—, —S—, —SO—, and —SO₂—.

The above alkyl, cycloalkyl, and alkenyl groups may each have a substituent. No particular limitation is imposed on the substituent, and examples thereof include halogen atoms (preferably a fluorine atom).

Ina compound PIIa-1 formed by replacing each of the organic cations represented by M₆₁ₐ⁺ and M_{61b}⁺ in formula (IIa-1) with H⁺, the acid dissociation constant a1-7 derived from an acidic moiety represented by A₆₁ₐH and the acid dissociation constant a1-8 derived from an acidic moiety represented by A_{61b}H each correspond to the acid dissociation constant a1 described above.

The compound PIIa-1 formed by replacing each of the cationic moieties M₆₁ₐ⁺ and M_{61b}⁺ in the structural moieties X in the formula (ha-1) corresponds to HA₆₁ₐ-L₆₁-N(R_{2X})-L₆₂-A_{61b}H. The compound PIIa-1 is the same as the acid generated from the compound represented by formula (IIa-1) upon irradiation with actinic rays or radiation.

At least one of M₆₁ₐ⁺, M_{61b}⁺, A₆₁ₐ⁻, A_{61b}⁻, L₆₁, L₆₂, or R_{2X} may have an acid-decomposable group as a substituent.

The definitions of A₇₁ₐ⁻, A_{71b}⁻, and A_{71c}⁻ in formula (IIa-2) are each the same as that of A₁₁⁻ in formula (Ia-1) above, and their preferred modes are also the same as that of A₁₁⁻. The definitions of M₇₁ₐ⁺, M_{71b}⁺, and M_{71c}⁺ are each the same as that of M₁₁⁺ in formula (Ia-1), and their preferred modes are also the same as that of M₁₁⁺.

The definitions of L₇₁, L₇₂, and L₇₃ in formula (IIa-2) are each the same as that of L₁ in formula (Ia-1) above, and their preferred modes are also the same as that of L₁.

In a compound PIIa-2 formed by replacing each of the organic cations represented by M₇₁ₐ⁺, M_{71b}⁺, and M_{71c}⁺ in formula (IIa-2) with H⁺, the acid dissociation constant a1-9 derived from an acidic moiety represented by A₇₁ₐH, the acid dissociation constant a1-10 derived from an acidic moiety represented by A_{71b}H, and the acid dissociation constant a1-11 derived from an acidic moiety represented by A_{71c}H each correspond to the acid dissociation constant a1 described above.

The compound PIIa-2 formed by replacing each of the cationic moieties M₇₁ₐ⁺, M_{71b}⁺, and M_{71c}⁺ in the structural moieties X in the formula (ha-1) with H⁺ corresponds to HA₇₁ₐ-L₇₁-N(L₇₃-A_{71c}H)-L₇₂-A_{71b}H. The compound PIIa-2 is the same as the acid generated from the compound represented by formula (IIa-2) upon irradiation with actinic rays or radiation.

At least one of M₇₁ₐ⁺, M_{71b}⁺, M_{71c}⁺, A₇₁ₐ⁻, A_{71b}⁻, A_{71c}⁻, L₇₁, L₇₂, or L₇₃ may have an acid-decomposable group as a substituent.

Examples of moieties that the compounds (1) to (2) can have and that differ from the cations are shown below.

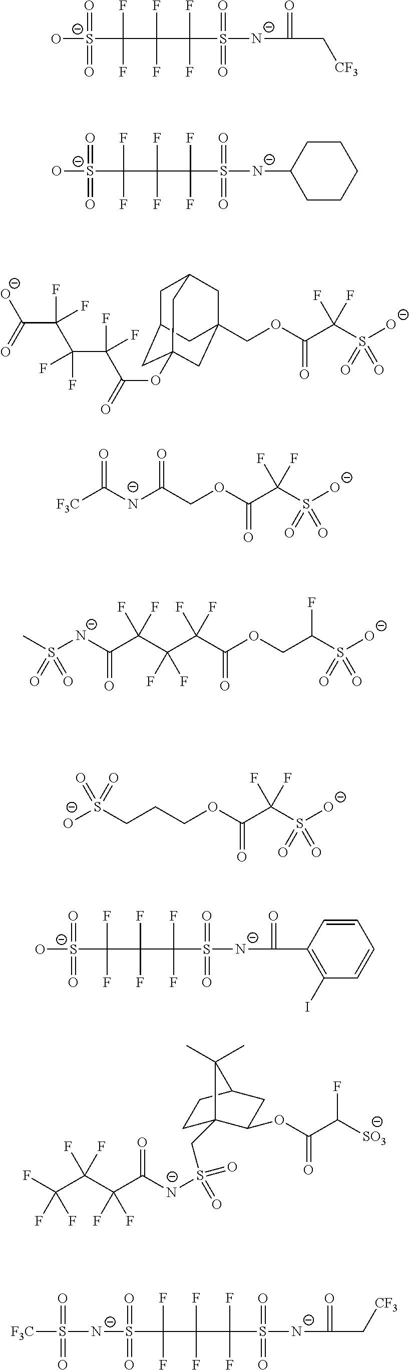

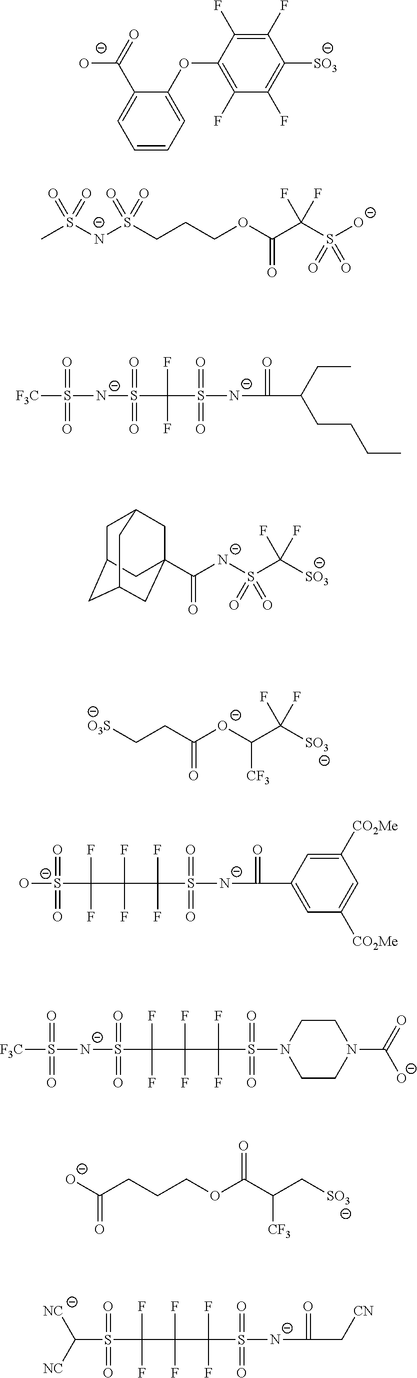

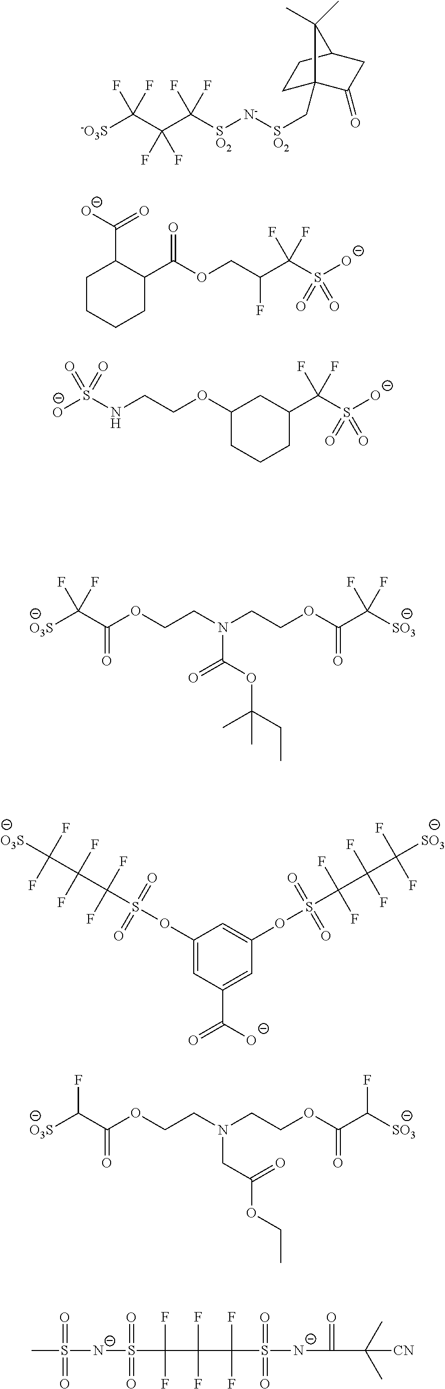

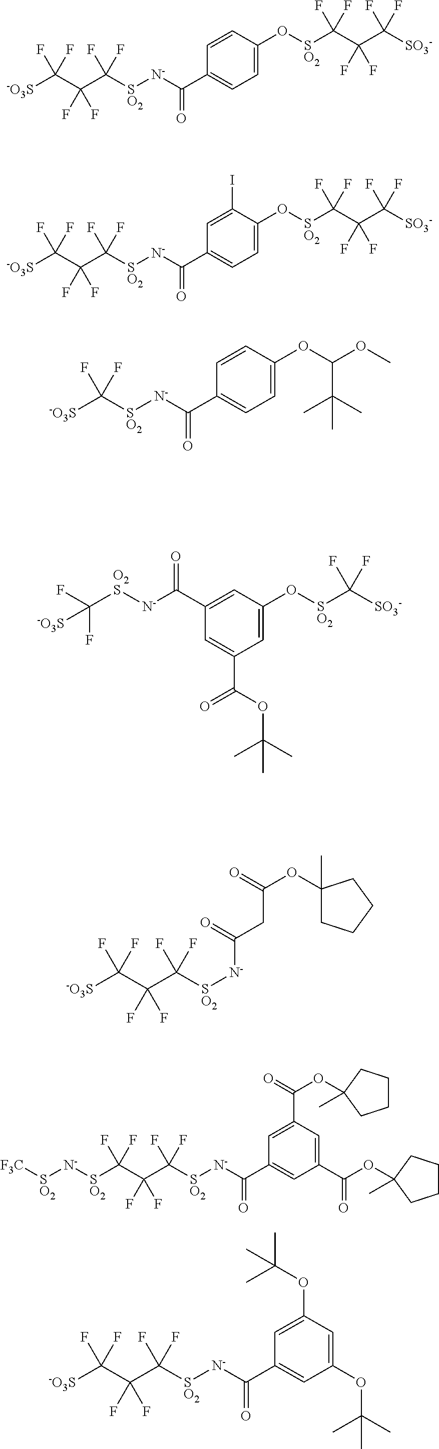

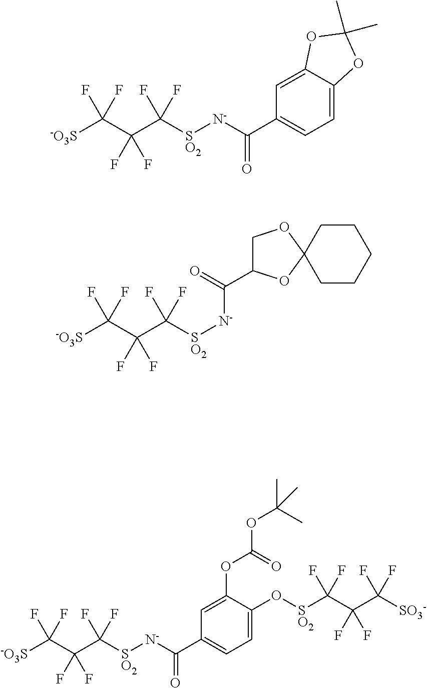

Specific examples of the photoacid generator are shown below, but the photoacid generator (B) is not limited thereto.

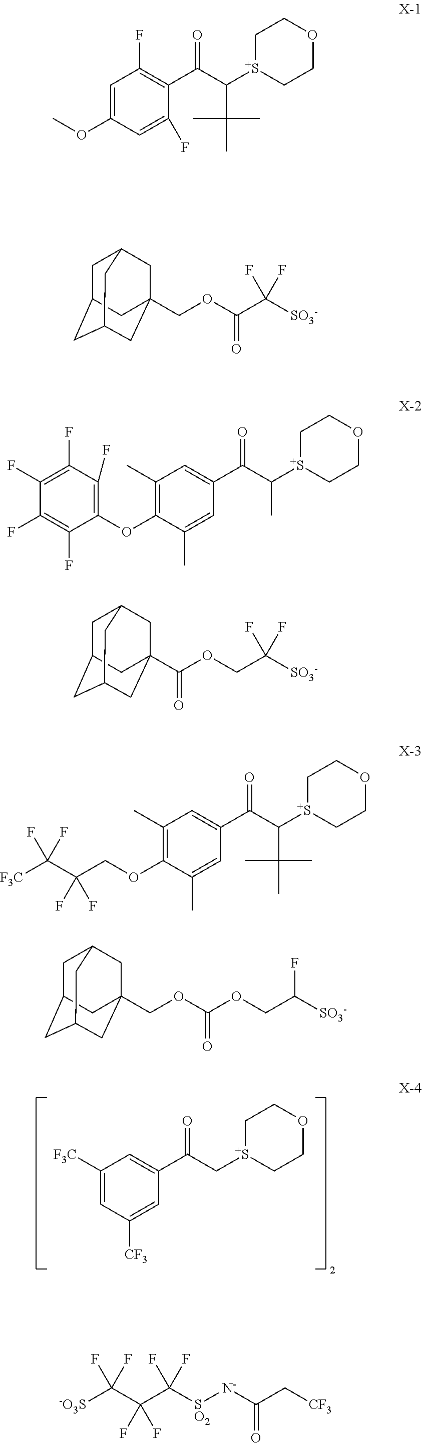

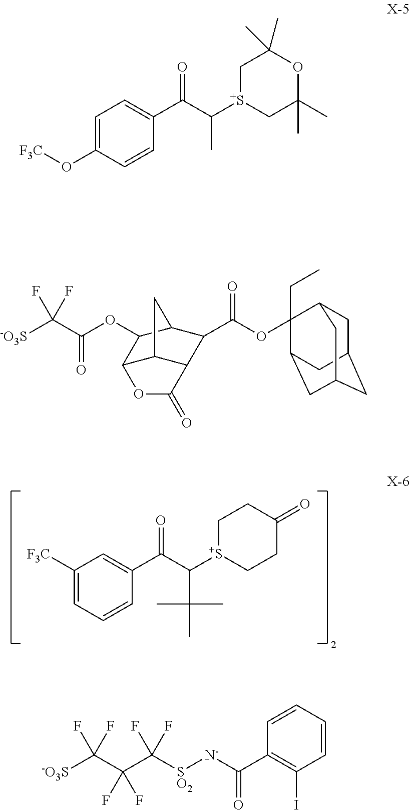

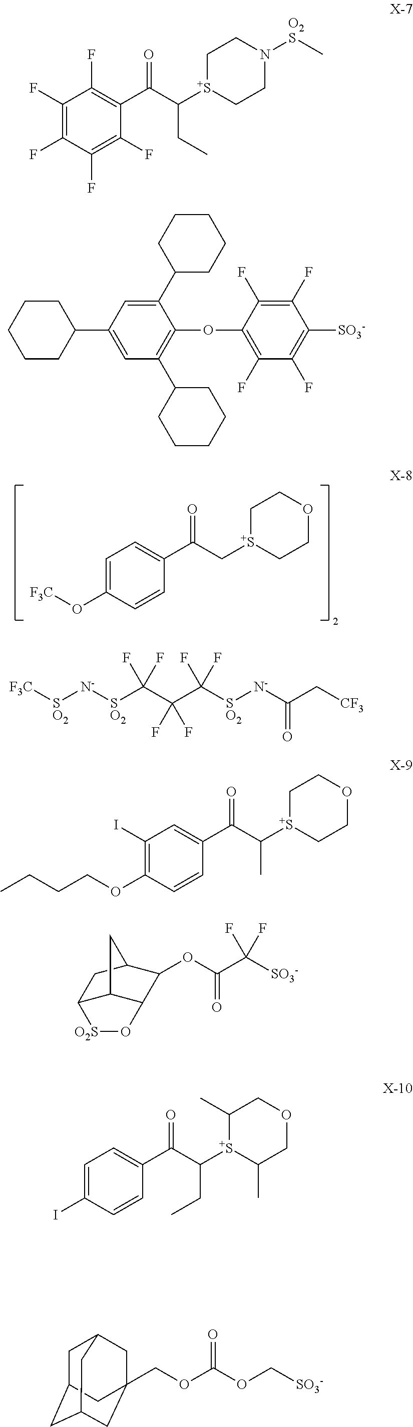

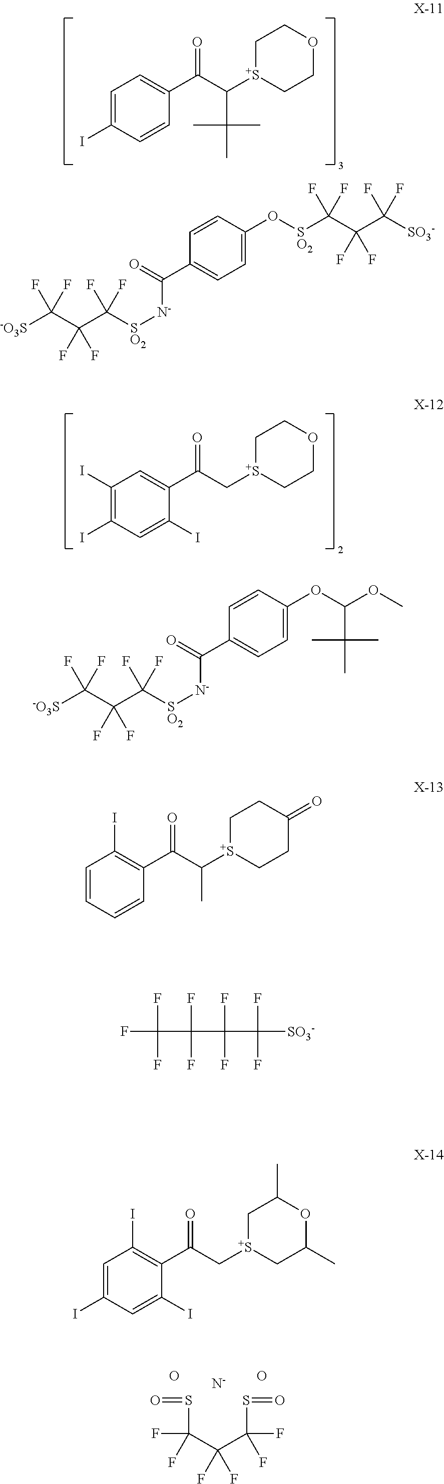

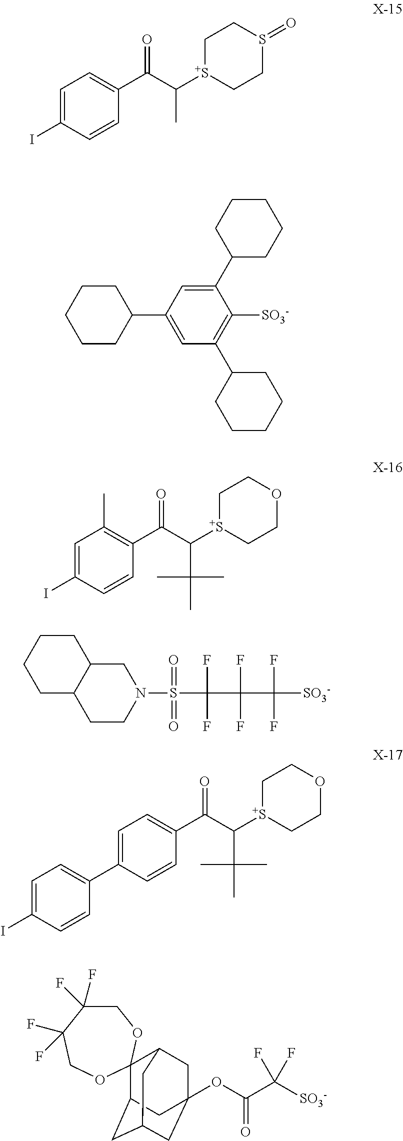

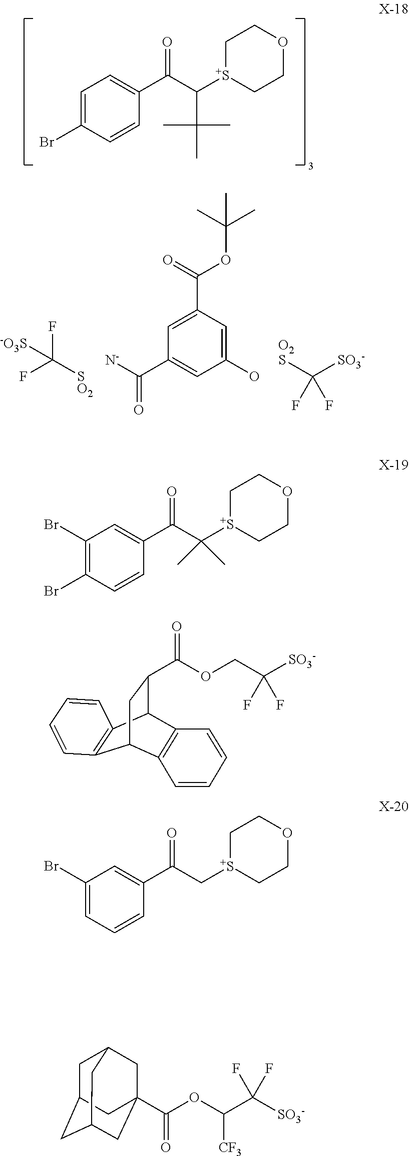

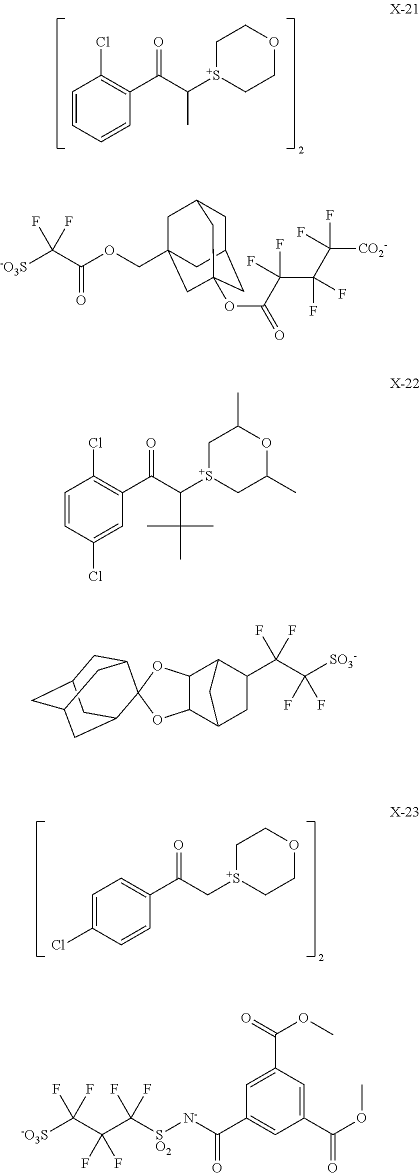

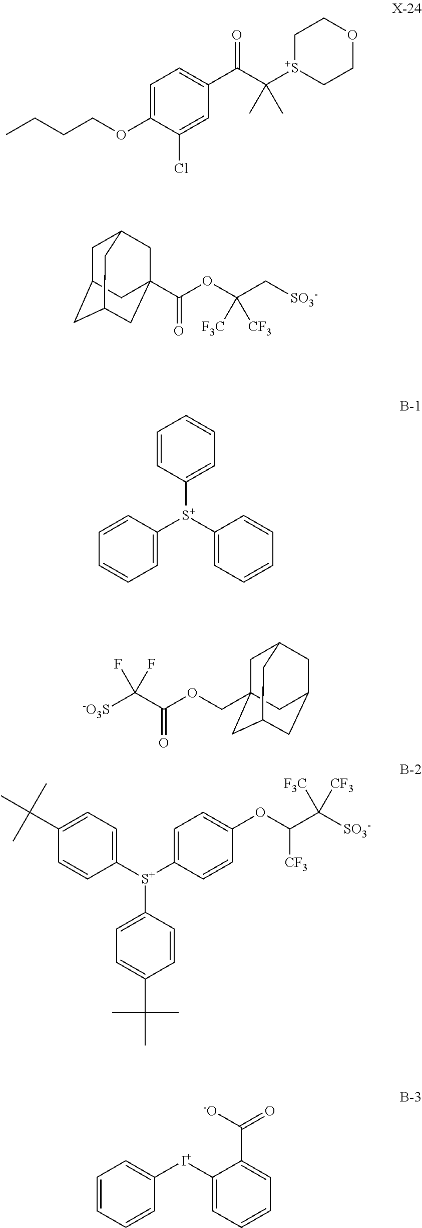

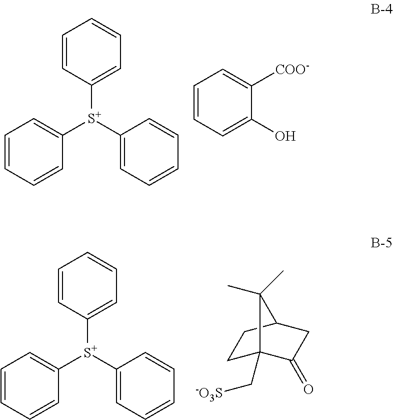

When the composition of the invention includes the photoacid generator (B), no particular limitation is imposed on the content of the photoacid generator (B). The content of the photoacid generator (B) with respect to the total amount of the solids in the composition is preferably 0.5% by mass or more and more preferably 1.0% by mass or more because the pattern to be formed can have a shaper rectangular cross-sectional shape. The content with respect to the total amount of the solids in the composition is preferably 50.0% by mass or less, more preferably 30.0% by mass or less, and still more preferably 25.0% by mass or less.

One photoacid generator (B) may be used alone, or two or more photoacid generators (B) may be used.

### <Acid Diffusion Control Agent>

The composition of the invention may include an acid diffusion control agent (C) that does not correspond to the compound (I).

The acid diffusion control agent functions as a quencher that traps the acid generated from the photoacid generator etc. during exposure to light to thereby suppress the reaction of the acid decomposable resin with an excess portion of the generated acid in unexposed portions.

No particular limitation is imposed on the acid diffusion control agent, and examples thereof include a basic compound (CA), a low-molecular weight compound (CB) having a nitrogen atom and having a group that leaves by the action of an acid, and a compound (CC) whose acid diffusion control ability decreases or disappears when the compound (CC) is irradiated with actinic rays or radiation.

Examples of the compound (CC) include an onium salt compound (CD) that serves as a weak acid weaker than the photoacid generator and a basic compound (CE) whose basicity decreases or disappears upon irradiation with actinic rays or radiation.

Specific examples of the basic compound (CA) include those described in paragraphs [132] to [0136] of WO2020/066824A, and specific examples of the basic compound (CE) whose basicity decreases or disappears upon irradiation with actinic rays or radiation include those described in paragraphs [0137] to [0155] of WO2020/066824A. Specific examples of the low-molecular weight compound (CB) having a nitrogen atom and having a group that leaves by the action of an acid include those described in paragraphs [0156] to [0163] of WO2020/066824A, and specific examples of an onium salt compound (CE) that has a nitrogen atom in its cationic moiety include those described in paragraph [0164] of WO2020/066824A.

Specific examples of the onium salt compound (CD) that serves as a weak acid weaker than the photoacid generator include those described in paragraphs [0305] to [0314] of WO2020/158337A.

In addition to the compounds described above, for example, known compounds disclosed in paragraphs [0627] to [0664] of US2016/0070167A, paragraphs [0095] to [0187] of US2015/0004544A, paragraphs [0403] to [0423] of US2016/0237190A, and paragraphs [0259] to [0328] of US2016/0274458A can be preferably used as the acid diffusion control agent.

When the composition of the invention includes the acid diffusion control agent, the content of the acid diffusion control agent (the total content when a plurality of acid diffusion control agents are present) with respect to the total amount of the solids in the composition is preferably 0.1 to 15.0% by mass and more preferably 1.0 to 15.0% by mass.

In the composition of the invention, one acid diffusion control agent may be used alone, or a combination of two or more acid diffusion control agents may be used.

### <Hydrophobic Resin>

The composition of the invention may further include a hydrophobic resin (D) different from the resin (A).

Preferably, the hydrophobic resin is designed so as to segregate on the surface of a resist film. However, it is not always necessary that, unlike a surfactant, the hydrophobic resin have a hydrophilic group in its molecule and contribute to uniform mixing of polar and nonpolar substances.

The effects of the addition of the hydrophobic resin include control of the static and dynamic contact angles of water on the surface of the resist film and reduction of outgassing.

From the viewpoint of segregation of the hydrophobic resin in a surface layer of the film, the hydrophobic resin has preferably at least one of a fluorine atom, a silicon atom, or a CH₃ partial structure included in a side chain portion of the resin and has more preferably two or more of them. Preferably, the hydrophobic resin has a hydrocarbon group having 5 or more carbon atoms. Each of these groups may be present as a substituent in the main chain of the resin or its side chain.

Examples of the hydrophobic resin include compounds described in paragraphs [0275] to [0279] of WO2020/004306A.

When the composition of the invention includes the hydrophobic resin, the content of the hydrophobic resin with respect to the total amount of the solids in the composition is preferably 0.01 to 20.0% by mass and more preferably 0.1 to 15.0% by mass.

### <Surfactant>

The composition of the invention may include a surfactant (E). When the surfactant is included, the compound has better adhesiveness, and a pattern with fewer development defects can be formed.

The surfactant is preferably a fluorine-based surfactant and/or a silicon-based surfactant.

Examples of the fluorine-based surfactant and/or the silicon-based surfactant include surfactants disclosed in paragraphs [0218] and [0219] of WO2018/193954A.

One of these surfactants may be used alone, or two or more of them may be used.

When the composition of the invention includes the surfactant, the content of the surfactant with respect to the total amount of the solids in the resist composition is preferably 0.0001 to 2.0% by mass, more preferably 0.0005 to 1.0% by mass, and still more preferably 0.1 to 1.0% by mass.

### <Solvent>

Preferably, the composition of the invention includes a solvent (F).

Preferably, the solvent includes at least one of (M1) propylene glycol monoalkyl ether carboxylate or (M2) at least one selected from the group consisting of propylene glycol monoalkyl ethers, lactates, acetates, alkoxypropionates, chain ketones, cyclic ketones, lactones, and alkylene carbonates. The solvent may further include a component other than the components (M1) and (M2).

The inventors have found that, when the above-described solvent and the above-described resin are used in combination, the coatability of the composition is improved, and a pattern with fewer development defects can be formed. The reason for this is unclear, but the inventors have considered that the reason may be as follows. The solubility of the resin in the above solvent, the boiling point of the solvent, and its viscosity are well-balanced, and therefore unevenness of the thickness of a resist film, the occurrence of precipitation during spin coating, etc. can be reduced.

The details of the components (M1) and (M2) are described in paragraphs [0218] to [0026] of WO2020/004306A, the contents of which are incorporated herein.

When the solvent further includes a component other than the components (M1) and (M2), the content of the component other than the components (M1) and (M2) with respect to the total amount of the solvent is preferably 5 to 30% by mass.

The content of the solvent in the composition of the invention is determined such that the concentration of the solids is preferably 0.5 to 30% by mass and more preferably 1 to 20% by mass. In this case, the coatability of the composition of the invention can be further improved.

The solids mean all the components other than the solvent and mean the components forming the actinic ray-sensitive or radiation-sensitive film, as described above.

The concentration of the solids is the mass percentage of the components other than the solvent with respect to the total mass of the composition of the invention.

The “total mass of the solids” is the total mass of the components obtained by removing the solvent from the total components of the composition of the invention. The solids are components other than the solvent as described above and may be, for example, solids or liquid at 25° C.

### <Additional Additives>

The composition of the invention may further include a dissolution inhibiting compound, a dye, a plasticizer, a photo sensitizer, a light absorber, and/or a compound capable of increasing the solubility in a developer (such as a phenol compound having a molecular weight of 1000 or less or an alicyclic or aliphatic compound including a carboxy group).

The composition of the invention may further include a dissolution inhibiting compound. The “dissolution inhibiting compound” is a compound that has a molecular weight of 3000 or less and is decomposed by the action of an acid to cause the degree of solubility of the composition of the invention in an organic-based developer to decrease.

The composition of the invention is preferably used as a photosensitive composition for EUV light.

The wavelength of the EUV light is 13.5 nm and is shorter than the wavelength of ArF light (wavelength: 193 nm) etc., and the number of incident photons when light exposure is performed at the same sensitivity is smaller. Therefore, the influence of “photon shot noise,” i.e., stochastic variations in the number of photons, is large, and this causes an increase in LER and bridge defects. One method to reduce the photon shot noise is to increase the exposure value to increase the number of incident photons, but there is a trade-off with a demand for higher sensitivity.

### [Applications]

The composition of the invention relates to an actinic ray-sensitive or radiation-sensitive resin composition that undergoes a reaction upon irradiation with actinic rays or radiation and changes its properties. More specifically, the composition of the invention relates to an actinic ray-sensitive or radiation-sensitive resin composition that is used for processes for manufacturing semiconductors such as ICs (Integrated Circuits), manufacturing of circuit boards for liquid crystals and thermal heads, production of imprint mold structures, other photofabrication processes, lithographic printing plates, and manufacturing of acid-curable compositions. The pattern formed in the invention can be used for etching processes, ion implantation processes, bump electrode forming processes, rewiring forming processes, MEMS (Micro Electro Mechanical Systems), etc.

The invention also relates to the following compound.

The compound represented by any of the following general formulas (IA)-1 to (IA)-3.

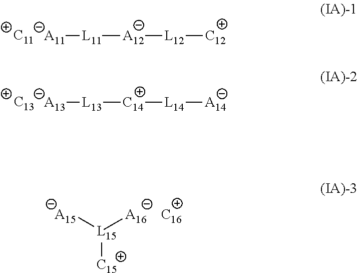

In general formulas (IA)-1 to (IA)-3,
A₁₁⁻ and A₁₃⁻ to A₁₆⁻ each independently represent an acid anionic group represented by formula (A-1) or (A-2) below.
A₁₂⁻ represents an acid anionic group represented by any of formulas (B-1) to (B-3) below.
C₁₁⁺ to C₁₆⁺ each independently represent a cationic group.
L₁₁ to L₁₄ each independently represent a divalent organic group.
L₁₅ represents a trivalent organic group.

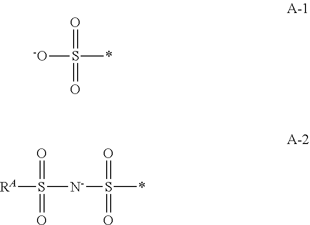

In formulas (A-1) to (A-2) above,
R^{A} represents an organic group.
* represents a bonding position.

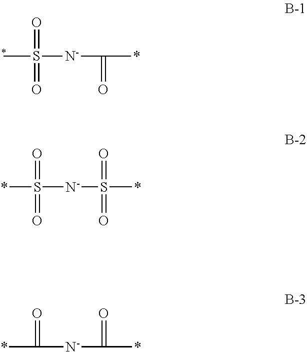 [SMILES: *C(=O)[N-]S(*)(=O)=O ; *S(=O)(=O)[N-]S(*)(=O)=O ; *C(=O)[N-]C(*)=O]

In formulas (B-1) to (B-3) above,

* represents a bonding position.

In general formulas (IA)-1 to (IA)-3, C₁₁⁺to C₁₆⁺, L₁₁ to L₁₄, and Lis have the same meanings as C₁₁⁺ to C₁₆⁺, L₁₁ to L₁₄, and L₁₅, respectively, in general formulas (I)-1 to (I)-3 above in the composition of the invention.

In formula (A-2), R^{A} has the same meaning as R^{A} in formula (A-2) above in the composition of the invention.

### <Actinic Ray-Sensitive or Radiation-Sensitive Film and Pattern Forming Method>

No particular limitation is imposed on the procedure of a pattern forming method using the above-described composition, but it is preferable that the pattern forming method includes the following steps.

Step 1: The step of forming an actinic ray-sensitive or radiation-sensitive film on a substrate using the actinic ray-sensitive or radiation-sensitive resin composition.

Step 2: The step of exposing the actinic ray-sensitive or radiation-sensitive film to light.

Step 3: The step of developing the exposed actinic ray-sensitive or radiation-sensitive film using a developer.

The procedure of each of the steps will next be described in detail.

### (Step 1: Step of Forming Actinic Ray-Sensitive or Radiation-Sensitive Film)

Step 1 is the step of forming an actinic ray-sensitive or radiation-sensitive film on a substrate using the composition of the invention.

Examples of the method for forming an actinic ray-sensitive or radiation-sensitive film on a substrate using the actinic ray-sensitive or radiation-sensitive resin composition include a method in which the actinic ray-sensitive or radiation-sensitive resin composition is applied to the substrate.

Preferably, the actinic ray-sensitive or radiation-sensitive resin composition is filtrated through a filter before the application as needed. The pore size of the filter is preferably 0.1 μm or less, more preferably 0.05 μm or less, and still more preferably 0.03 μm or less. The filer is preferably a polytetrafluoroethylene-made filter, a polyethylene-made filter, or a nylon-made filter.

The actinic ray-sensitive or radiation-sensitive resin composition can be applied to a substrate (e.g., a silicon substrate or a silicon dioxide coating) used for production of an integrated circuit element using an appropriate application method using a spinner, a coater, etc. The application method is preferably spin coating using a spinner. The number or revolutions when the spin coating using a spinner is performed is preferably 1000 to 3000 rpm.

After the application of the actinic ray-sensitive or radiation-sensitive resin composition, the substrate may be dried to thereby form the resist film. If necessary, an undercoat film (an inorganic film, an organic film, or an antireflection film) may be formed as an underlayer of the resist film.

Examples of the drying method include a method in which the substrate is heated and dried. The heating may be performed using heating means included in an ordinary exposing device and/or an ordinary developing device or may be performed using a hot plate etc. The heating temperature is preferably 80 to 150° C., more preferably 80 to 140° C., and still more preferably 80 to 130° C. The heating time is preferably 30 to 1000 seconds, more preferably 60 to 800 seconds, and still more preferably 60 to 600 seconds.

No particular limitation is imposed on the film thickness of the actinic ray-sensitive or radiation-sensitive film (typically a resist film), but the film thickness is preferably 10 to 120 nm because a finer pattern can be formed with higher accuracy.

In particular, when the actinic ray-sensitive or radiation-sensitive film is exposed to EUV light, the film thickness of the actinic ray-sensitive or radiation-sensitive film is more preferably 10 to 65 nm and still more preferably 15 to 50 nm. When ArF liquid immersion exposure is performed, the film thickness of the actinic ray-sensitive or radiation-sensitive film is more preferably 10 to 120 nm and still more preferably 15 to 90 nm.

A topcoat may be formed on the actinic ray-sensitive or radiation-sensitive film using a topcoat composition.

It is preferable that the topcoat composition is immiscible with the actinic ray-sensitive or radiation-sensitive film and can be uniformly applied to the upper surface of the actinic ray-sensitive or radiation-sensitive film. No particular limitation is imposed on the topcoat, and a well-known topcoat can be formed using a well-known method. For example, the topcoat can be formed using a method described in paragraphs [0072] to [0082] of JP2014-059543A.

It is preferable, for example, that a topcoat including a basic compound described in JP2013-61648A is formed on the actinic ray-sensitive or radiation-sensitive film. Specific examples of the basic compound that can be included in the topcoat include basic compounds that can be included in the actinic ray-sensitive or radiation-sensitive resin composition.

It is also preferable that the topcoat includes a compound including at least one group or bond selected from the group consisting of an ether bond, a thioether bond, a hydroxy group, a thiol group, a carbonyl bond, and an ester bond.

### (Step 2: Exposure Step)

Step 2 is the step of exposing the actinic ray-sensitive or radiation-sensitive film to light.

Examples of the light exposure method include a method in which the actinic ray-sensitive or radiation-sensitive film formed is irradiated with actinic rays or radiation through a prescribed mask.

Examples of the actinic rays or radiation include infrared rays, visible rays, ultraviolet rays, far-ultraviolet rays, extreme ultraviolet rays, X rays, and electron beams. Far-ultraviolet rays having a wavelength of preferably 250 nm or shorter, more preferably 220 nm or shorter, and particularly preferably 1 to 200 nm are preferred, and specific examples include KrF excimer laser light (248 nm), ArF excimer laser light (193 nm), F₂ excimer laser light (157 nm), EUV light (13 nm), X rays, and electron beams.

It is preferable to perform baking (heating) after the light exposure but before development. The baking facilitates the reaction in the exposed portions, and the sensitivity and the pattern shape are further improved.

The heating temperature is preferably 80 to 150° C., more preferably 80 to 140° C., and still more preferably 80 to 130° C.

The heating time is preferably 10 to 1000 seconds, more preferably 10 to 180 seconds, and still more preferably 30 to 120 seconds.

The heating may be performed using heating means included in an ordinary exposing device and/or an ordinary developing device or may be performed using a hot plate etc.

This step is referred to as post-exposure baking.

### (Step 3: Developing Step)

Step 3 is the step of developing the exposed actinic ray-sensitive or radiation-sensitive film with a developer to form a pattern.

The developer may be an alkali developer or may be a developer including an organic solvent (hereinafter referred to as an organic-based developer).

Examples of the developing method include: a method in which the substrate is dipped into a bath filled with the developer for a prescribed time (a dipping method); a method in which the developer is placed on the surface of the substrate so as to bulge due to surface tension and left to stand for a prescribed time to develop the resist film (a puddle method); a method in which the developer is sprayed onto the surface of the substrate (a spraying method); and a method in which the developer is continuously discharged from a developer discharging nozzle onto the substrate rotating at a constant speed while the developer discharging nozzle is scanned at a constant speed (a dynamic dispensing method).

The step of replacing the solvent with another solvent to stop the development may be performed after the developing step.

No particular limitation is imposed on the developing time so long as the resin in unexposed portions is dissolved sufficiently, and the developing time is preferably 10 to 300 seconds and more preferably 20 to 120 seconds.

The temperature of the developer is preferably 0 to 50° C. and more preferably 15 to 35° C.

The alkali developer used is preferably an aqueous alkali solution including an alkali. No particular limitation is imposed on the type of aqueous alkali solution. Examples of the aqueous alkali solution include aqueous alkali solutions including quaternary ammonium salts typified by tetramethylammonium hydroxide, inorganic alkalis, primary amines, secondary amines, tertiary amines, alcohol amines, cyclic amines, etc. In particular, the alkali developer is preferably an aqueous solution of a quaternary ammonium salt typified by tetramethylammonium hydroxide (TMAH). An appropriate amount of an alcohol, a surfactant, etc. may be added to the alkali developer. The alkali concentration of the alkali developer is generally 0.1% to 20% by mass. The pH of the alkali developer is generally 10.0 to 15.0.

The organic-based developer is preferably a developer including at least one organic solvent selected from the group consisting of ketone-based solvents, ester-based solvents, alcohol-based solvents, amide-based solvents, ether-based solvents, and hydrocarbon-based solvents.

A mixture of a plurality of solvents selected from the above solvents may be used, or the organic-based developer may be mixed with water or a solvent other that the above solvents. The content of water with respect to the total mass of the developer is preferably less than 50% by mass, more preferably less than 20% by mass, and still more preferably less than 10% by mass, and it is particularly preferable that the developer includes substantially no water.

The content of the organic solvent with respect to the total mass of the organic-based developer is preferably from 50% by mass to 100% by mass inclusive, more preferably from 80% by mass to 100% by mass inclusive, still more preferably from 90% by mass to 100% by mass inclusive, and particularly preferably from 95% by mass to 100% by mass inclusive.

### (Additional Steps)

Preferably, the above pattern forming method further includes the step of, after step 3, washing with a rinsing solution.

Examples of the rinsing solution used in the rinsing step after the step of developing using the alkali developer include pure water. An appropriate amount of a surfactant may be added to the pure water.

An appropriate amount of a surfactant may be added to the rinsing solution.

No particular limitation is imposed on the rinsing solution used for the rinsing step after the step of developing using the alkali developer so long as the rinsing solution does not dissolve the pattern, and a solution including a general-purpose organic solvent can be used. Preferably, the rinsing solution used includes at least one organic solvent selected from the group consisting of hydrocarbon-based solvents, ketone-based solvents, ester-based solvents, alcohol-based solvents, amide-based solvents, and ether-based solvents.

No particular limitation is imposed on the method for the rinsing step, and examples thereof include: a method in which the rinsing solution is continuously discharged onto the substrate rotating at a constant speed (a spin coating method); a method in which the substrate is dipped into a bath filled with the rinsing solution for a prescribed time (a dipping method); and a method in which the rinsing solution is sprayed onto the surface of the substrate (a spraying method).

The pattern forming method of the invention may further include a heating (post-baking) step after the rinsing step. Through this step, the developer and the rinsing solution remaining between traces of the pattern and inside the pattern are removed by baking. Through this step, the resist pattern is annealed, and the effect of improving surface roughness of the pattern is obtained. The heating step after the rinsing step is performed at generally 40 to 250° C. (preferably 90 to 200° C.) for generally 10 seconds to 3 minutes (preferably 30 seconds to 120 seconds).

The pattern formed may be used as a mask to perform etching treatment on the substrate. Specifically, the pattern formed in step 3 may be used as a mask to process the substrate (or the underlayer film and the substrate) to thereby form a pattern on the substrate.

No particular limitation is imposed on the method for processing the substrate (or the underlayer film and the substrate). It is preferable that the pattern formed in step 3 is used as a mask and the substrate (or the underlayer film and the substrate) is dry-etched to form a pattern on the substrate. The dry etching is preferably oxygen plasma etching.

Preferably, the composition of the invention and various materials (such as the solvent, the developer, the rinsing solution, a composition for forming an antireflection film, and the composition for forming the topcoat) used in the pattern forming method of the invention include no impurities such as metals. The content of the impurities included in each of these materials is preferably 1 ppm by mass or less, more preferably 10 ppb by mass or less, still more preferably 100 ppt by mass or less, particularly preferably 10 ppt by mass or less, and most preferably 1 ppt by mass or less. No particular limitation is imposed on the lower limit of the content of the impurities, and the content is preferably 0 ppt by mass or more. Examples of the metal impurities include Na, K, Ca, Fe, Cu, Mg, Al, Li, Cr, Ni, Sn, Ag, As, Au, Ba, Cd, Co, Pb, Ti, V, W, and Zn.

Examples of a method for removing impurities such as metals from the above materials include filtration using a filter. The details of the filtration using a filer are described in paragraph [0321] of WO2020/004306A.

Examples of a method for reducing the amount of impurities such as metals included in the above materials include: a method in which raw materials including smaller amounts of metals are used as the raw materials forming the above materials; a method in which the raw materials forming the above materials are filtrated through a filter; and a method in which distillation is performed under the condition that contamination is reduced as much as possible, for example, by coating the inside of the device used with Teflon (registered trademark).

Besides the filtration using a filter, an adsorbent may be used to remove impurities. The filtration using a filter and the absorbent may be used in combination. The adsorbent used may be a well-known adsorbent, and examples of the adsorbent that can be used include inorganic-based adsorbents such as silica gel and zeolite and organic-based adsorbents such as activated carbon. To reduce the amount of impurities such as metals included in the above materials, it is necessary to prevent the metal impurities from mixing in the production process. Whether the metal impurities have been sufficiently removed from the production device can be checked by measuring the content of metal components included in a washing solution used to clean the production device. The content of the metal components included in the washing solution after use is preferably 100 ppt (parts per trillion) by mass or less, more preferably 10 ppt by mass or less, and still more preferably 1 ppt by mass or less. No particular limitation is imposed on the lower limit of the content, and the content is preferably 0 ppt by mass or more.

An electrically conductive compound may be added to an organic treatment solution such as the rinsing solution in order to prevent failure of chemical solution pipes and various parts (such as filters, O-rings, and tubes) due to electrostatic charges and subsequent electrostatic discharge. No particular limitation is imposed on the electrically conductive compound, and examples thereof include methanol. No particular limitation is imposed on the amount of the electrically conductive compound added. From the viewpoint of maintaining preferred development characteristics or rinsing characteristics, the amount of the electrically conductive compound is preferably 10% by mass or less and more preferably 5% by mass or less. No particular limitation is imposed on the lower limit, and the amount of the electrically conductive compound is preferably 0.01% by mass or more.

The chemical solution pipes used may be, for example, SUS (stainless steel) pipes or pipes coated with antistatic-treated polyethylene, antistatic-treated polypropylene, or an antistatic-treated fluorocarbon resin (such as polytetrafluoroethylene or a perfluoroalkoxy resin). Similarly, antistatic-treated polyethylene, antistatic-treated polypropylene, or an antistatic-treated fluorocarbon resin (such as polytetrafluoroethylene or a perfluoroalkoxy resin) may be used for the filters and the O-rings.

### <Method for Manufacturing Electronic Device>

The present invention also relates to a method for manufacturing an electronic device including the pattern forming method described above and to an electronic device manufactured by the manufacturing method.

In preferred modes of the electronic device of the invention, the device is installed in electric and electronic devices (such as household electrical appliances and OA (Office Automation) devices, media-related devices, optical devices, and telecommunication devices).

### EXAMPLES

The present invention will be further described in detail by way of Examples. Materials, amounts used, ratios, treatment details, treatment procedures, etc. shown in the following Examples can be appropriately changed so long as they do not depart from the gist of the invention. Therefore, the scope of the present invention should not be construed as limited to the following Examples.

### <Resins (A)>

The structures of repeating units of the resins (A) used, their compositional ratios (% molar ratios), their weight average molecular weights (Mw), and their disparities (Mw/Mn) are shown below.

The compositional ratios of the repeating units in the resins (A) used (their molar % ratios; in the order from left to right), their weight average molecular weights (Mw), and their disparities (Mw/Mn) are also shown.

The weight average molecular weight (Mw) and dispersity (Mw/Mn) of each resin (A) were measured by GPC (solvent: tetrahydrofuran (THF)). The compositional ratios (molar % ratios) of each resin were measured by ¹³C-NMR (nuclear magnetic resonance).

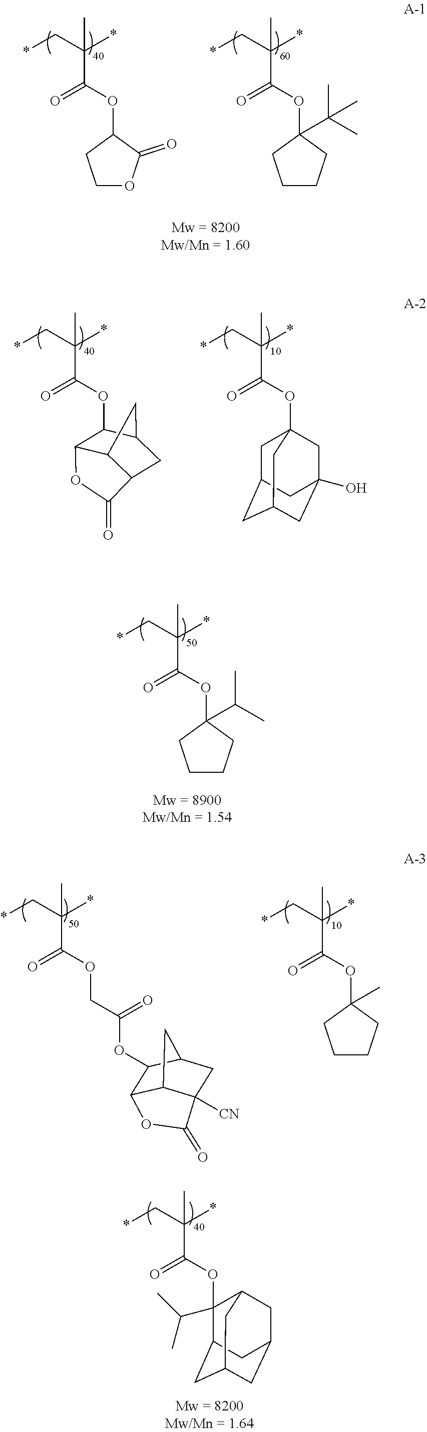

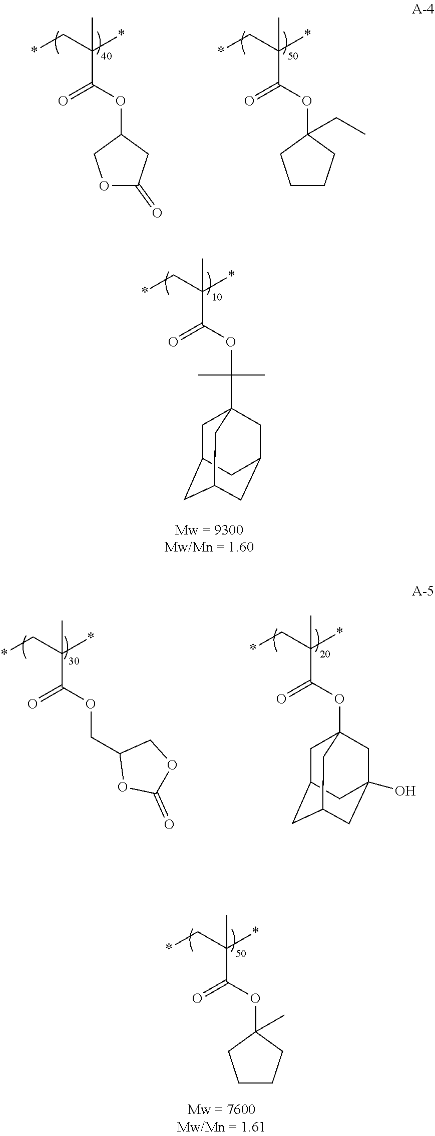

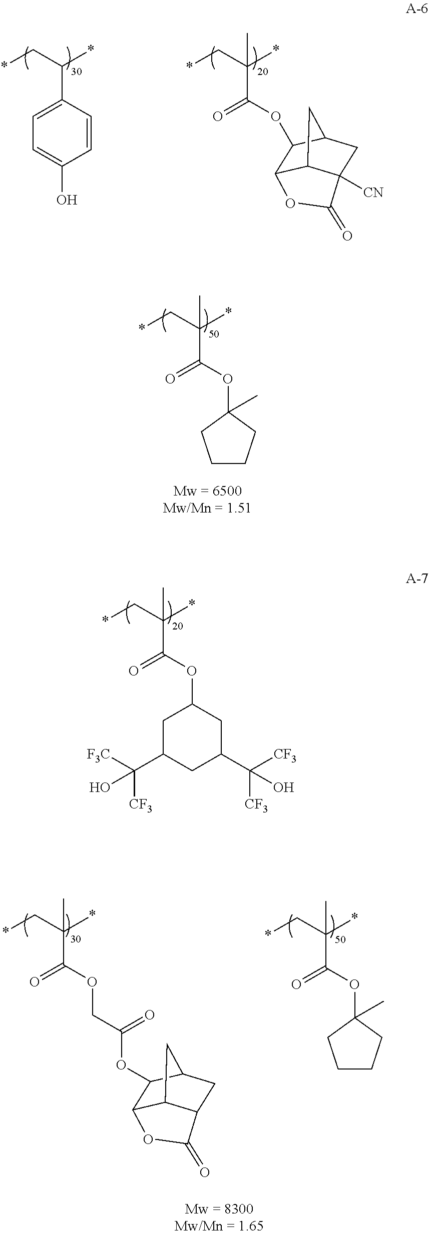

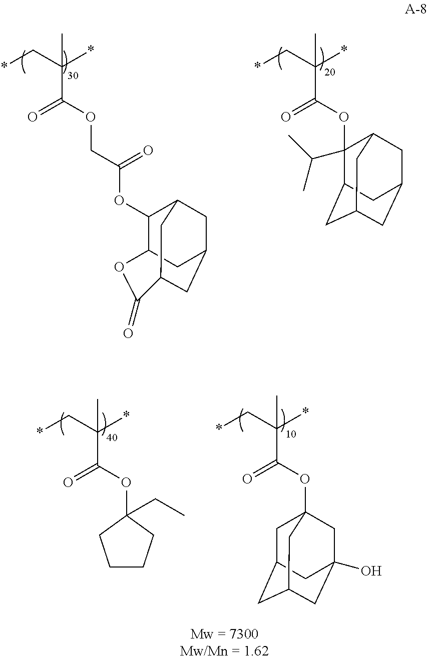

### Compound (I)

### (Synthesis Example 1) Synthesis of Compound X-1

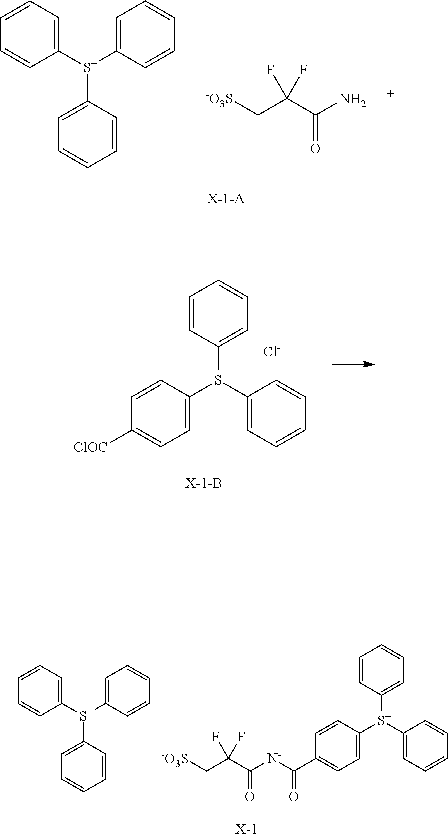

A 300 mL three-neck flask was charged with 10.0 g (22.9 mmol) of compound (X-1-A), 100 mL of acetonitrile, and 5.9 g (45.7 mmol) of diisopropylethylamine, and the mixture was cooled to 0° C. Then 8.7 g (22.9 mmol) of compound (X-1-B) was slowly added to the mixture, and the resulting mixture was allowed to react for 2 hours. 200 mL of methylene chloride and 100 mL of water were added to the mixture, and the aqueous layer was removed. Then the organic layer was washed twice with 100 mL of water, and the solvent was removed by evaporation under reduced pressure. The crude product was purified by silica gel column chromatography (eluted with a chloroform/methanol solvent mixture) to thereby obtain 11.6 g of compound (X-1) as a white solid (yield: 70%).

¹H-NMR (300 MHz, deuterated dimethyl sulfoxide): 6 (ppm) 7.81 (m, 29H)

¹⁹F-NMR (300 MHz, deuterated dimethyl sulfoxide): −109.03 (2F)

### (Synthesis Example 2) Synthesis of Compound X-8

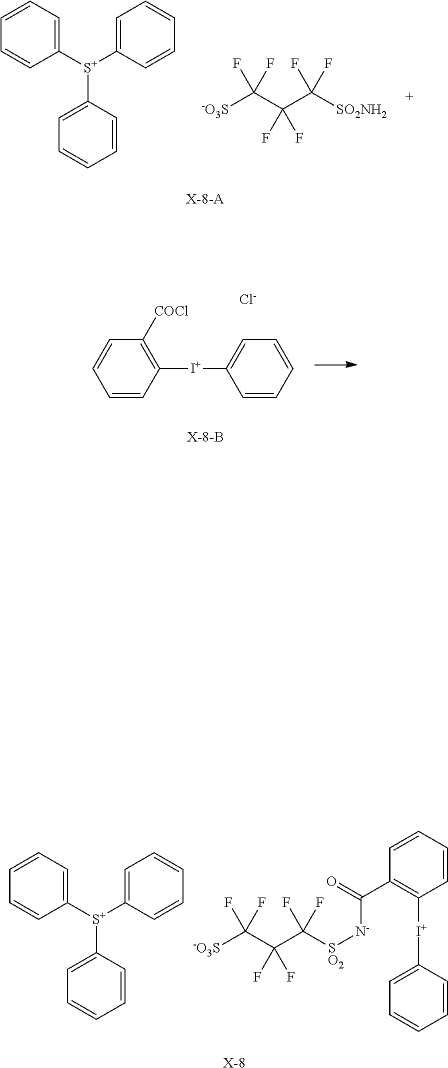

A 300 mL three-neck flask was charged with 10.0 g (17.4 mmol) of compound (X-8-A), 100 mL of acetonitrile, and 4.5 g (34.9 mmol) of diisopropylethylamine, and the mixture was cooled to 0° C. Then 6.6 g (22.9 mmol) of compound (X-8-B) was slowly added to the mixture, and the resulting mixture was allowed to react for 2 hours. Then 200 mL or methylene chloride and 100 mL of water were added, and the aqueous layer was removed. Then the organic layer was washed twice with 100 mL or water, and the solvent was removed by evaporation under reduced pressure. The crude product was purified by silica gel column chromatography (eluted with a chloroform/methanol solvent mixture) to thereby obtain 10.4 g of compound (X-8) as a white solid (yield: 68%).

¹H-NMR (300 MHz, deuterated dimethyl sulfoxide): 6 (ppm) 6.81 (d, 1H), 7.66 (m, 4H), 7.83 (m, 16H), 8.29 (m, 3H)

¹⁹F-NMR (300 MHz, deuterated dimethyl sulfoxide): −118.69 (2F), −114.11 (2F), −113.13 (2F)

The same procedure as in Synthesis Examples 1 and 2 was repeated to synthesize compounds (X-2) to (X-7) and compounds (X-9) to (X-16). The structures of compounds (X-1) to (X-16) are shown below.

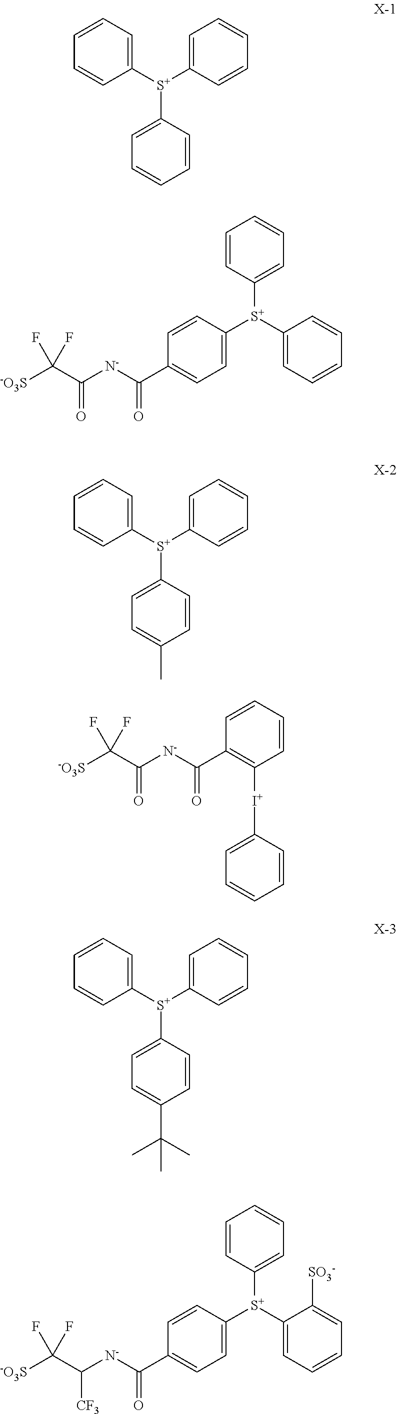

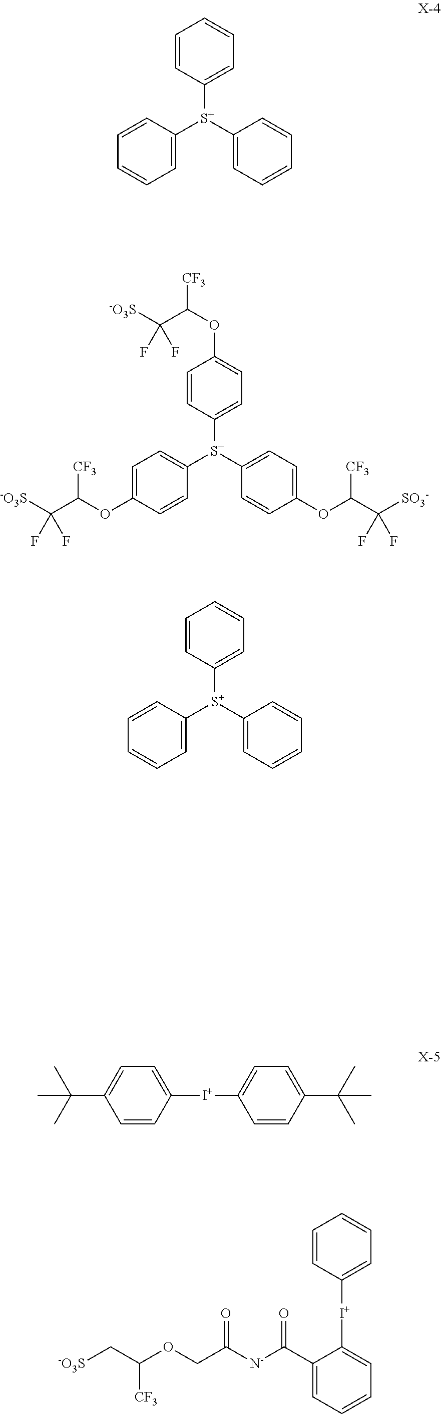

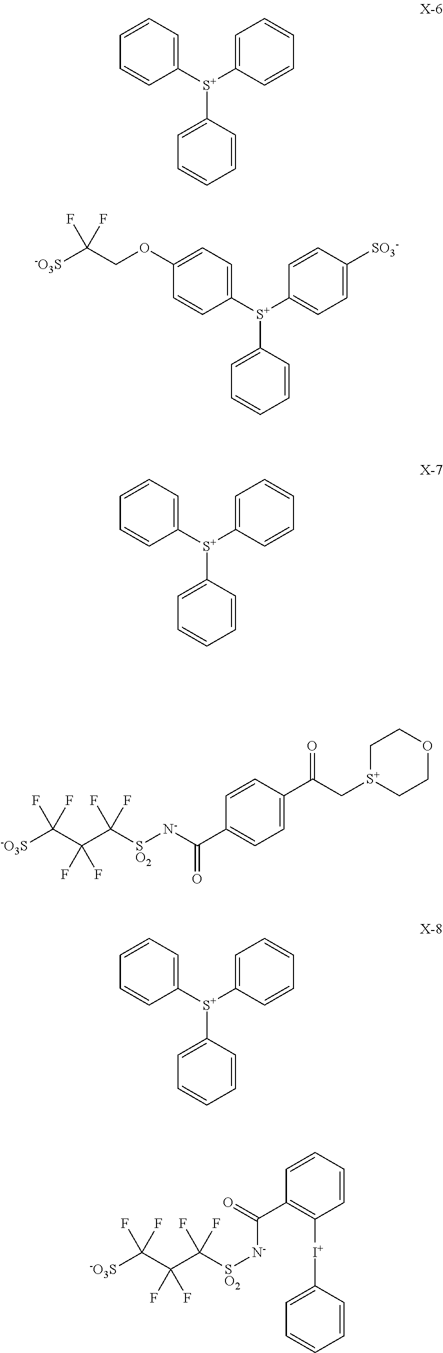

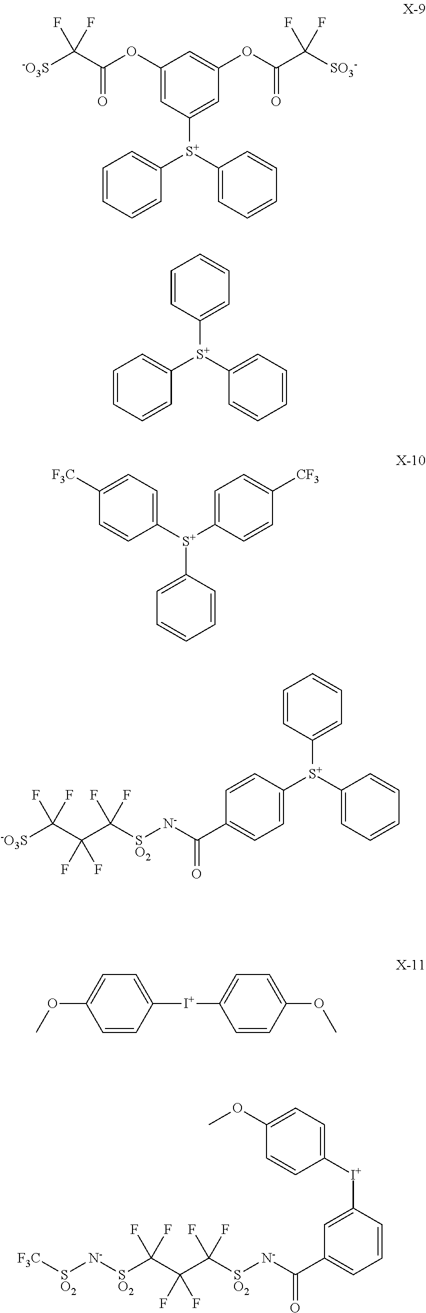

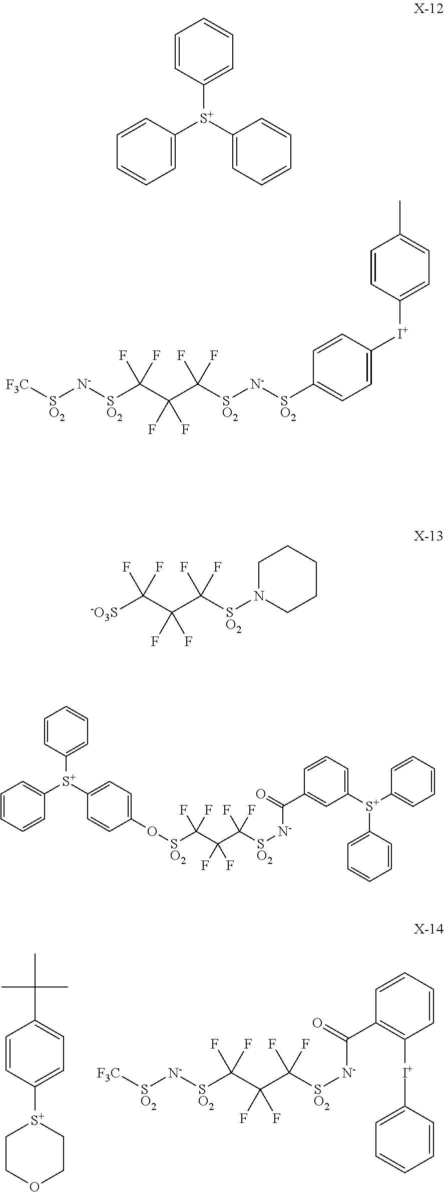

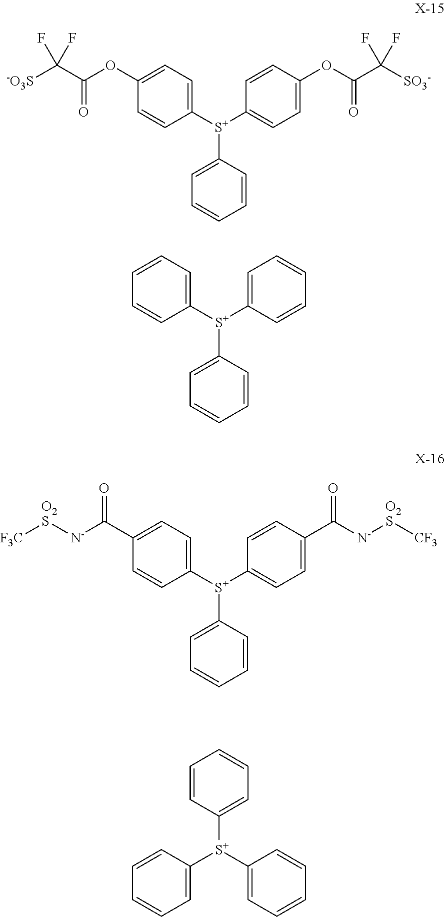

(Acid Dissociation Constants pKa of Acid Generated from Compound (I))

Table 1 shows the acid dissociation constants pKa of the acid generated from each compound (I).

The acid dissociation constants pKa of the acid generated form each compound (I) were measured as follows. Specifically, for a compound obtained by replacing an acid anionic group in one of the compounds X-1 to X-16 with its corresponding acid group, the software package 1 available from ACD/Labs was used to determine a value based on a Hammett substituent constant and a database of known literature values by computation in the manner described above. When the pKa could not be computed using the above method, a value obtained using Gaussian 16 based on the DFT (density functional theory) was used.

In the Table below, “pKa1” is the acid dissociation constant at the first stage, and “pKa2” is the acid dissociation constant at the second stage. “pKa3” is the acid dissociation constant at the third stage. The smaller the pKa value, the higher the acidity.

As described above, the compounds X-1 to X-3, X-5 to X-8, and X-10 to X-14 each correspond to the compound (I) described above. pKa1 corresponds to the acid dissociation constant a1 described above, and pKa2 corresponds to the acid dissociation constant a2.

As described above, the compound X-4 corresponds to the compound (I) described above. pKa1 corresponds to the acid dissociation constant a1, and pKa2 corresponds to the acid dissociation constant a2. pKa3 corresponds to the acid dissociation constant a3.

The acid generated from compound X-4 (a compound formed by replacing two sulfonium cations in the compound X-4 with H⁺ and adding H⁺ to one SO₃⁻) has a symmetrical structure. Therefore, the acid dissociation constants pKa of the acid groups derived from the three acid anionic groups are theoretically the same.

These are denoted as “pKa1,” “pKa2,” and “pKa3” for convenience.

As described above, the compounds X-9, X-15, and X-16 each correspond to the compound (I). pKa1 corresponds to the acid dissociation constant a1, and pKa2 corresponds to the acid dissociation constant a2.

In each compound, the acid dissociation constants pKa of acid groups derived from two acid anionic groups are theoretically the same.

These are denoted as “pKa1” and “pKa2” for convenience.

TABLE 1

| | Number of linked ions | | | | |
|---|---|---|---|---|---|
| | | Anionic | | | |
| Compound | Cationic group | group | pKa1 | pKa2 | pKa3 |
| X-1 | 1 | 2 | −3.57 | 6.39 | |
| X-2 | 1 | 2 | −4.37 | 7.8 | |
| X-3 | 1 | 2 | −3.19 | −1.95 | |
| X-4 | 1 | 3 | −3.09 | −3.09 | −3.09 |
| X-5 | 1 | 2 | 0.83 | 8.88 | |
| X-6 | 1 | 2 | −2.7 | −1.93 | |
| X-7 | 1 | 2 | −3.42 | −0.92 | |
| X-8 | 1 | 2 | −3.42 | −0.51 | |
| X-9 | 1 | 2 | −3.57 | −3.57 | |
| X-10 | 1 | 2 | −3.43 | −1.84 | |
| X-11 | 1 | 2 | −10.89 | −0.63 | |
| X-12 | 1 | 2 | −10.74 | −5.07 | |
| X-13 | 2 | 1 | −3.27 | −1.52 | |
| X-14 | 1 | 2 | −10.89 | −0.64 | |
| X-15 | 1 | 2 | −3.37 | −3.37 | |
| X-16 | 1 | 2 | −4.6 | −4.6 | |

In Table 1, the number of linked ions is the number of cationic groups or the number of anionic groups in a chain including the zwitterionic group in the compound (I).

### <Photoacid Generator (B)>

The structures of photoacid generators used that do not correspond to the compound (I) are shown below.

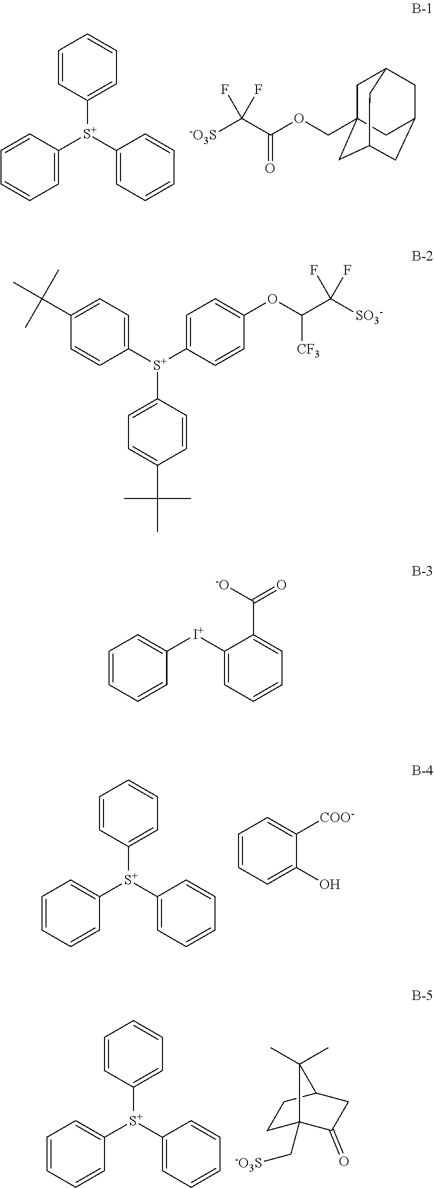

### <Acid Diffusion Control Agent>

The structures of acid diffusion control agents used that do not correspond to the compound (I) are shown below.

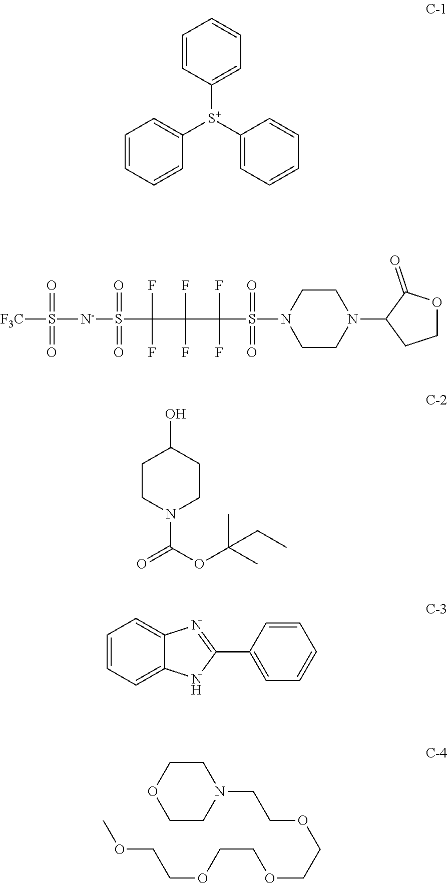 [SMILES: c1ccc([S+](c2ccccc2)c2ccccc2)cc1 ; O=C1OCCC1N1CCN(S(=O)(=O)C(F)(F)C(F)(F)C(F)(F)S(=O)(=O)[N-]S(=O)(=O)C(F)(F)F)CC1 ; CCC(C)(C)OC(=O)N1CCC(O)CC1 ; c1ccc(-c2nc3ccccc3[nH]2)cc1 ; COCCOCCOCCOCCN1CCOCC1]

### <Hydrophobic Resin>

The structures of repeating units in hydrophobic resins used, their compositional ratios (molar % ratios), their weight average molecular weights (Mw), and their disparities (Mw/Mn) are shown below.

The compositional ratios of the repeating units in the hydrophobic resins used (their molar % ratios; in the order from left to right), their weight average molecular weights (Mw), and their disparities (Mw/Mn) are also shown.

The weight average molecular weight (Mw) and dispersity (Mw/Mn) of each hydrophobic resin were measured by GPC (solvent: tetrahydrofuran (THF)). The compositional ratios (molar % ratios) of each resin were measured by ¹³C-NMR (nuclear magnetic resonance).

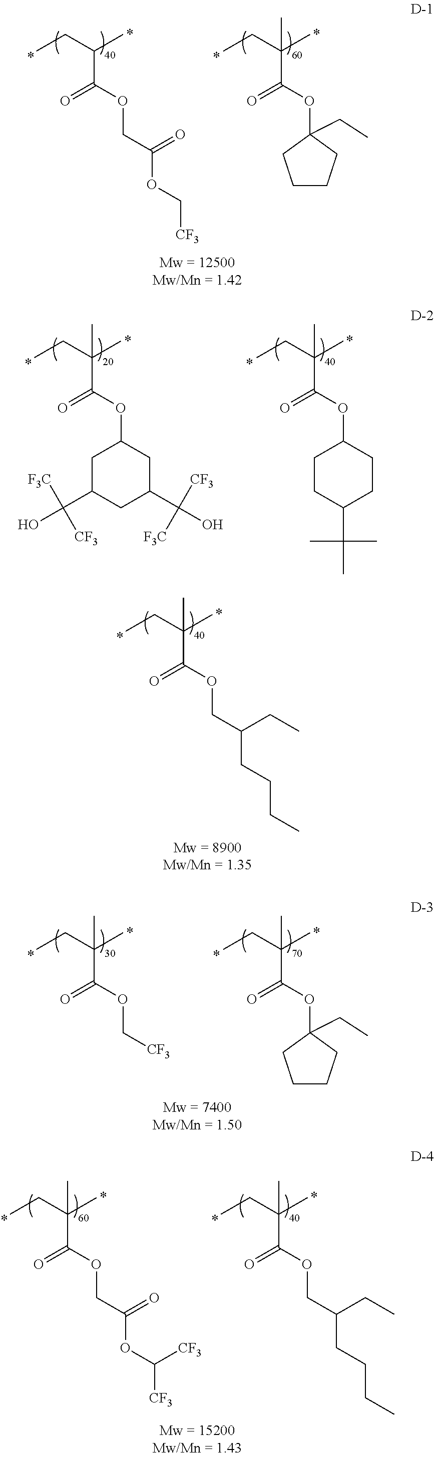

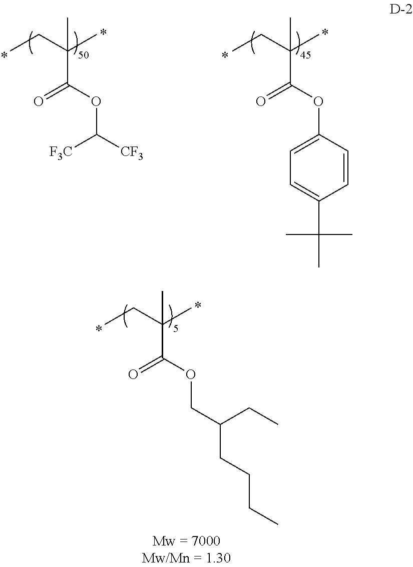

### <Surfactant>

The surfactant used was E-1 below.

E-1: PolyFox PF-6320 (manufactured by OMNOVA Solutions Inc.: fluorine based)

### <Solvents>

The solvents used are shown below.
F-1: Propylene glycol monomethyl ether acetate (PGMEA: 1-methoxy-2-acetoxypropane)
F-2: Propylene glycol monomethyl ether (PGME: 1-methoxy-2-propanol)
F-3: Cyclohexanone
F-4: γ-Butyrolactone
F-5: Ethyl lactate

### Examples 1-1 to 1-21, 2-1 to 2-21, 3-1 to 3-14, and 4-1 to 4-14 and Comparative Examples 1-1 to 1-3, 2-1 to 2-3, 3-1 to 3-3, and 4-1 to 4-3

### <Preparation of Resist Compositions> (ArF Exposure)

### Examples 1-1 to 1-21 and 2-1 to 2-21 and Comparative Examples 1-1 to 1-3 and 2-1 to 2-3

Components shown in Table 2 were dissolved in a solvent shown in Table 2 to prepare a solution with a solid concentration of 4.0% by mass. The solution was filtered using a polyethylene filter having a pore size of 0.02 μm to thereby prepare a resist composition.

The solids mean all the components other than the solvent. The resist compositions obtained were used for Examples and Comparative Examples.

In the Table, each “% by mass” column represents the content (% by mass) of a compound with respect to the total amount of the solids in a resist composition. In the Table, the amount (parts by mass) of each solvent used was listed.

### <Pattern Forming Method (1): ArF Exposure, Alkali Development (Positive)>

One of the resist compositions shown in Table 2 immediately after the manufacturing was applied to a 6-inch Si wafer pre-treated with hexamethyldisilazane (HMDS) using a spin coater Mark 8 manufactured by Tokyo Electron Ltd. and dried on a hot plate at 100° C. for 60 seconds to thereby obtain a resist film having a film thickness of 90 nm. Here, one inch is 0.0254 m.

The wafer with the resist film formed thereon was subjected to pattern exposure through an exposure mask using an ArF excimer laser scanner (PAS 5500/1500 manufactured by ASML, wavelength: 193 nm, NA: 0.50). Then the wafer was baked at a temperature of 115° C. for 60 seconds, then developed using a 2.38% by mass aqueous tetramethylammonium hydroxide solution (TMAHaq) for 30 seconds, rinsed with pure water, and then spin-dried. A 1:1 line-and-space resist pattern with a line width of 50 nm was thereby obtained.

### <Performance Evaluation>

### [Scum]

A cross section of each of the 1:1 line-and-space patterns with a line width of 50 nm was observed under a scanning electron microscope (SEM S-9380II manufactured by Hitachi, Ltd.), and the presence or absence of scum was evaluated using a two-level (A and B) evaluation system.
A: No scum was visually observed.
B: Scum was visually observed.

### [Preservation Stability]

A cross-sectional shape of each of the patterns was observed under a scanning electron microscope (SEM S-9380II manufactured by Hitachi, Ltd.). The exposure amount when the 1:1 line-and-space resist pattern with a line width of 50 nm was resolved was used as sensitivity (Eop).

The resist compositions were stored at room temperature (23° C.) for one month, and then 1:1 line-and-space resist patterns with a line width of 50 nm were formed using the same procedure as above. For each of the resist patterns, the exposure amount when the resist pattern was resolved was used as the sensitivity (Eop). The difference between the sensitivity when the composition immediately after the manufacturing was used and the sensitivity when the composition stored for one month at room temperature after the manufacturing was used {|(the exposure amount when the pattern formed using the composition stored at room temperature for one month was resolved—the exposure amount when the pattern formed using the composition immediately after the manufacturing was resolved)|} was evaluated according to the following evaluation criteria.
A: The difference in sensitivity was less than 1 mJ/cm².
B: The difference in sensitivity was 1 mJ/cm² or more and less than 3 mJ/cm².
C: The difference in sensitivity was 3 mJ/cm² or more.

### [Pattern Shape]

A cross section of each of the 1:1 line-and-space patterns with a line width of 50 nm was observed under a scanning electron microscope (SEM S-9380II manufactured by Hitachi, Ltd.). The pattern line width Lb of the resist pattern at the bottom and the pattern line width La of the resist pattern at the top were measured, and the pattern shape was evaluated using a four-level (A, B, C, and D) evaluation system.
A: (Lb/La)≤1.03
B: 1.03<(Lb/La)≤1.06
C: 1.06<(Lb/La)≤1.1
D: 1.1<(Lb/La)

### <Pattern Forming Method (2): ArF Exposure, Alkali Development (Negative)>

One of the resist compositions shown in Table 2 immediately after the manufacturing was applied to a 6-inch Si wafer pre-treated with hexamethyldisilazane (HMDS) using a spin coater Mark 8 manufactured by Tokyo Electron Ltd. and dried on a hot plate at 100° C. for 60 seconds to thereby obtain a resist film having a film thickness of 90 nm. Here, one inch is 0.0254 m.

The wafer with the resist film formed thereon was subjected to pattern exposure through an exposure mask using an ArF excimer laser scanner (PAS 5500/1500 manufactured by ASML, wavelength: 193 nm, NA: 0.50). Then the wafer was baked at a temperature of 115° C. for 60 seconds, then developed using n-butyl acetate for 30 seconds, and spin-dried. A 1:1 line-and-space resist pattern with a line width of 50 nm was thereby obtained.

### <Performance Evaluation>

### [Scum]

A cross section of each of the 1:1 line-and-space patterns with a line width of 50 nm was observed under a scanning electron microscope (SEM S-9380II manufactured by Hitachi, Ltd.), and the presence or absence of scum was evaluated using a two-level (A and B) evaluation system.
A: No scum was visually observed.
B: Scum was visually observed.

### [Preservation Stability]

A cross-sectional shape of each of the patterns was observed under a scanning electron microscope (SEM S-9380II manufactured by Hitachi, Ltd.). The exposure amount when the 1:1 line-and-space resist pattern with a line width of 50 nm was resolved was used as sensitivity (Eop).

The resist compositions were stored at room temperature (23° C.) for one month, and then 1:1 line-and-space resist patterns with a line width of 50 nm were formed using the same procedure as above. For each of the resist patterns, the exposure amount when the resist pattern was resolved was used as the sensitivity (Eop). The difference between the sensitivity when the composition immediately after the manufacturing was used and the sensitivity when the composition stored for one month at room temperature after the manufacturing was used {|(the exposure amount when the pattern formed using the composition stored at room temperature for one month was resolved—the exposure amount when the pattern formed using the composition immediately after the manufacturing was resolved)|} was evaluated according to the following evaluation criteria.
A: The difference in sensitivity was less than 1 mJ/cm².
B: The difference in sensitivity was 1 mJ/cm² or more and less than 3 mJ/cm².
C: The difference in sensitivity was 3 mJ/cm² or more.

### [Pattern Shape]

A cross section of each of the 1:1 line-and-space patterns with a line width of 50 nm was observed under a scanning electron microscope (SEM S-9380II manufactured by Hitachi, Ltd.). The pattern line width Lb of the resist pattern at the bottom and the pattern line width La of the resist pattern at the top were measured, and the pattern shape was evaluated using a four-level (A, B, C, and D) evaluation system.
A: (Lb/La)≤1.03
B: 1.03<(Lb/La)≤1.06
C: 1.06<(Lb/La)≤1.1
D: 1.1<(Lb/La)

### <Preparation of Resist Compositions> (EUV Exposure)

### Examples 3-1 to 3-14 and 4-1 to 4-14 and Comparative Examples 3-1 to 3-3 and 4-1 to 4-4

Components shown in Table 3 were dissolved in a solvent shown in Table 3 to prepare a solution with a solid concentration of 2.0% by mass. The solution was filtered using a polyethylene filter having a pore size of 0.02 μm to thereby prepare a resist composition.

The solids mean all the components other than the solvent. The resist compositions obtained were used for Examples and Comparative Examples.

In the Table, each “% by mass” column represents the content (% by mass) of a compound with respect to the total amount of the solids in a resist composition. In the Table, the amount (parts by mass) of each solvent used was listed.

### <Pattern Forming Method (3): EUV Exposure, Alkali Development (Positive)>

A composition AL412 (manufactured by Brewer Science) for formation of an underlayer film was applied to a silicon wafer and baked at 205° C. for 60 seconds to form an underlayer film having a film thickness of 20 nm. A resist composition shown in Table 3 immediately after the manufacturing was applied to the underlayer film and baked at 100° C. for 60 seconds to form a resist film having a film thickness of 30 nm.

An EUV exposure device (Micro Exposure Tool manufactured by Exitech, NA 0.3, Quadrupole, outer sigma: 0.68, inner sigma: 0.36) was used to subject the obtained silicon wafer having the resist film to pattern irradiation. The reticle used was a mask with a line size of 50 nm and a line:space ratio of 1:1.

The resist film exposed to light was baked at 90° C. for 60 seconds, developed with an aqueous tetramethylammonium hydroxide solution (2.38% by mass) for 30 seconds, and rinsed with pure water for 30 second. The resulting resist film was spin-dried to thereby obtain a 1:1 line-and-space resist pattern with a line width of 50 nm.

### <Performance Evaluation>

### [Scum]

A cross section of each of the 1:1 line-and-space patterns with a line width of 50 nm was observed under a scanning electron microscope (SEM S-9380II manufactured by Hitachi, Ltd.), and the presence or absence of scum was evaluated using a two-level (A and B) evaluation system.
A: No scum was visually observed.
B: Scum was visually observed.

### [Preservation Stability]

A cross-sectional shape of each of the patterns was observed under a scanning electron microscope (SEM S-9380II manufactured by Hitachi, Ltd.). The exposure amount when the 1:1 line-and-space resist pattern with a line width of 50 nm was resolved was used as sensitivity (Eop).

The resist compositions were stored at room temperature (23° C.) for one month, and then 1:1 line-and-space resist patterns with a line width of 50 nm were formed using the same procedure as above. For each of the resist patterns, the exposure amount when the resist pattern was resolved was used as the sensitivity (Eop). The difference between the sensitivity when the composition immediately after the manufacturing was used and the sensitivity when the composition stored for one month at room temperature after the manufacturing was used {|(the exposure amount when the pattern formed using the composition stored at room temperature for one month was resolved—the exposure amount when the pattern formed using the composition immediately after the manufacturing was resolved)|} was evaluated according to the following evaluation criteria.
A: The difference in sensitivity was less than 1 mJ/cm².
B: The difference in sensitivity was 1 mJ/cm² or more and less than 3 mJ/cm².
C: The difference in sensitivity was 3 mJ/cm² or more.

### [Pattern Shape]

A cross section of each of the 1:1 line-and-space patterns with a line width of 50 nm was observed under a scanning electron microscope (SEM S-9380II manufactured by Hitachi, Ltd.). The pattern line width Lb of the resist pattern at the bottom and the pattern line width La of the resist pattern at the top were measured, and the pattern shape was evaluated using a four-level (A, B, C, and D) evaluation system.
A: (Lb/La)≤1.03
B: 1.03<(Lb/La)≤1.06
C: 1.06<(Lb/La)≤1.1
D: 1.1<(Lb/La)

### <Pattern Forming Method (4): EUV Exposure, Organic Solvent Development (Negative)>

A composition AL412 (manufactured by Brewer Science) for formation of an underlayer film was applied to a silicon wafer and baked at 205° C. for 60 seconds to form an underlayer film having a film thickness of 20 nm. A resist composition shown in Table 3 immediately after the manufacturing was applied to the underlayer film and baked at 100° C. for 60 seconds to form a resist film having a film thickness of 30 nm.

An EUV exposure device (Micro Exposure Tool manufactured by Exitech, NA 0.3, Quadrupole, outer sigma: 0.68, inner sigma: 0.36) was used to subject the obtained silicon wafer having the resist film to pattern irradiation. The reticle used was a mask with a line size of 50 nm and a line:space ratio of 1:1.

The resist film exposed to light was baked at 90° C. for 60 seconds, developed with n-butyl acetate for 30 seconds, and spin-dried to thereby obtain a 1:1 line-and-space resist pattern with a line width of 50 nm.

### <Performance Evaluation>

### [Scum]

A cross section of each of the 1:1 line-and-space patterns with a line width of 50 nm was observed under a scanning electron microscope (SEM S-9380II manufactured by Hitachi, Ltd.), and the presence or absence of scum was evaluated using a two-level (A and B) evaluation system.
A: No scum was visually observed.
B: Scum was visually observed.

### [Preservation Stability]

A cross-sectional shape of each of the patterns was observed under a scanning electron microscope (SEM S-9380II manufactured by Hitachi, Ltd.). The exposure amount when the 1:1 line-and-space resist pattern with a line width of 50 nm was resolved was used as sensitivity (Eop).

The resist compositions were stored at room temperature (23° C.) for one month, and then 1:1 line-and-space resist patterns with a line width of 50 nm were formed using the same procedure as above. For each of the resist patterns, the exposure amount when the resist pattern was resolved was used as the sensitivity (Eop). The difference between the sensitivity when the composition immediately after the manufacturing was used and the sensitivity when the composition stored for one month at room temperature after the manufacturing was used {|(the exposure amount when the pattern formed using the composition stored at room temperature for one month was resolved—the exposure amount when the pattern formed using the composition immediately after the manufacturing was resolved)|} was evaluated according to the following evaluation criteria.
A: The difference in sensitivity was less than 1 mJ/cm².
B: The difference in sensitivity was 1 mJ/cm² or more and less than 3 mJ/cm².
C: The difference in sensitivity was 3 mJ/cm² or more.

### [Pattern Shape]

A cross section of each of the 1:1 line-and-space patterns with a line width of 50 nm was observed under a scanning electron microscope (SEM S-9380II manufactured by Hitachi, Ltd.). The pattern line width Lb of the resist pattern at the bottom and the pattern line width La of the resist pattern at the top were measured, and the pattern shape was evaluated using a four-level (A, B, C, and D) evaluation system.
A: (Lb/La)≤1.03
B: 1.03<(Lb/La)≤1.06
C: 1.06<(Lb/La)≤1.1
D: 1.1<(Lb/La)

The evaluation results obtained are shown in Tables 2 and 3.

TABLE 2

| | | | | | | | Acid | | | | | | | | | | |
|---|---|---|---|---|---|---|---|---|---|---|---|---|---|---|---|---|---|
| | | | | | Photoacid | | diffusion | | | | | | | | | | |
| | | | | | generator | | control | | Hydrophobic | | Surfactant | | | | Performance | | |
| | Compound (I) | | Resin A | | B | | agent C | | resin D | | E | | Solvent F | | | Preser- | |
| | | % by | | % by | | % by | | % by | | % by | | % by | | Mass | | vation | Pattern |
| | Type | mass | Type | mass | Type | mass | Type | mass | Type | mass | Type | mass | Type | ratio | Scum | stability | shape |
| Example 1-1 | X-1 | 20.0 | A-1 | 75.8 | — | — | — | — | D-1 | 4.2 | — | — | F-1/F-2 | 80/20 | A | B | A |
| Example 1-2 | X-1 | 18.5 | A-5 | 80.4 | — | — | — | — | D-2 | 1.1 | — | — | F-1/F-4 | 90/10 | A | B | A |
| Example 1-3 | X-2 | 15.5 | A-2 | 82.4 | — | — | — | — | D-2 | 2.1 | — | — | F-1/F- | 70/25/5 | A | B | A |
| | | | | | | | | | | | | | 2/F-3 | | | | |
| Example 1-4 | X-2 | 16.0 | A-4 | 82.9 | — | — | — | — | D-3 | 1.1 | — | — | F-1/F-2 | 70/30 | A | B | A |
| Example 1-5 | X-3 | 14.5 | A-3 | 82.7 | — | — | — | — | D-5 | 2.8 | — | — | F-1/F- | 70/25/5 | A | C | B |
| | | | | | | | | | | | | | 2/F-5 | | | | |
| Example 1-6 | X-4 | 10.0 | A-2 | 86.1 | B-4 | 3.0 | — | — | D-4 | 0.9 | — | — | F-1/F-2 | 80/20 | A | A | C |
| Example 1-7 | X-5 | 25.0 | A-1 | 74.1 | — | — | — | — | D-1 | 0.9 | — | — | F-1/F-2 | 80/20 | A | B | A |
| Example 1-8 | X-5 | 22.0 | A-8 | 72.4 | — | — | — | — | D-1 | 5.5 | E-1 | 0.1 | F-1/F-2 | 40/60 | A | B | A |
| Example 1-9 | X-6 | 16.0 | A-2 | 82.5 | — | — | — | — | D-5 | 1.5 | — | — | F-1/F-2 | 90/10 | A | C | B |
| Example 1-10 | X-7 | 17.0 | A-4 | 79.2 | — | — | — | — | D-2 | 3.8 | — | — | F-1/F-2 | 70/30 | A | A | A |
| Example 1-11 | X-8 | 16.0 | A-1 | 81.1 | — | — | — | — | D-5 | 2.9 | — | — | F-1/F-3 | 90/10 | A | A | A |
| Example 1-12 | X-8 | 18.0 | A-4 | 77.8 | — | — | C-1 | 1.0 | D-5 | 3.2 | — | — | F-1/F-2 | 80/20 | A | A | A |
| Example 1-13 | X-9 | 10.0 | A-1 | 84.9 | — | — | C-2 | 2.0 | D-3 | 3.1 | — | — | F-1/F-2 | 50/50 | A | A | C |
| Example 1-14 | X-10 | 22.0 | A-1 | 76.7 | — | — | — | — | D-5 | 1.3 | — | — | F-1/F-2 | 80/20 | A | A | B |
| Example 1-15 | X-10 | 20.0 | A-4 | 75.7 | B-1 | 3.0 | — | — | D-5 | 1.3 | — | — | F-1/F-4 | 50/50 | A | A | B |
| Example 1-16 | X-11 | 18.0 | A-4 | 81.1 | — | — | — | — | D-4 | 0.9 | — | — | F-1/F-2 | 70/30 | A | A | A |
| Example 1-17 | X-12 | 17.0 | A-3 | 79.9 | — | — | — | — | D-5 | 3.1 | — | — | F-1/F-2 | 70/30 | A | A | A |
| Example 1-18 | X-13 | 15.0 | A-2 | 84.1 | — | — | — | — | D-4 | 0.9 | — | — | F-1/F-2 | 80/20 | A | C | C |
| Example 1-19 | X-14 | 18.0 | A-5 | 80.1 | — | — | C-3 | 1.0 | D-1 | 0.9 | — | — | F-1/F-4 | 85/15 | A | A | A |
| Example 1-20 | X-15 | 15.0 | A-2 | 79.5 | — | — | — | — | D-3 | 5.5 | — | — | F-1/F- | 70/25/5 | A | A | C |
| | | | | | | | | | | | | | 2/F-3 | | | | |
| Example 1-21 | X-16 | 5.0 | A-8 | 76.5 | B-1 | 15.0 | C-4 | 0.5 | D-1 | 2.9 | E-1 | 0.1 | F-1/F-2 | 80/20 | A | A | C |
| Comparative | — | — | A-2 | 72.2 | B-1 | 20.0 | C-4 | 5.0 | D-1 | 2.8 | — | — | F-1/F-2 | 80/20 | A | C | D |
| Example 1-1 | | | | | | | | | | | | | | | | | |
| Comparative | — | — | A-4 | 74.9 | B-2 | 20.0 | C-4 | 4.0 | D-2 | 1.1 | — | — | F-1/F-2 | 70/30 | B | C | D |
| Example 1-2 | | | | | | | | | | | | | | | | | |
| Comparative | — | — | A-3 | 73.8 | B-1 | 5.0 | — | — | D-2 | 1.2 | — | — | F-1/F-2 | 80/20 | B | D | D |
| Example 1-3 | | | | | B-3 | 20.0 | | | | | | | | | | | |
| Example 2-1 | X-1 | 20.0 | A-1 | 75.8 | — | — | — | — | D-1 | 4.2 | — | — | F-1/F-2 | 80/20 | A | B | A |
| Example 2-2 | X-1 | 18.5 | A-5 | 80.4 | — | — | — | — | D-2 | 1.1 | — | — | F-1/F-4 | 90/10 | A | B | A |
| Example 2-3 | X-2 | 15.5 | A-2 | 82.4 | — | — | — | — | D-2 | 2.1 | — | — | F-1/F- | 70/25/5 | A | B | A |
| | | | | | | | | | | | | | 2/F-3 | | | | |
| Example 2-4 | X-2 | 16.0 | A-4 | 82.9 | — | — | — | — | D-3 | 1.1 | — | — | F-1/F-2 | 70/30 | A | B | A |
| Example 2-5 | X-3 | 14.5 | A-3 | 82.7 | — | — | — | — | D-5 | 2.8 | — | — | F-1/F- | 70/25/5 | A | C | B |
| | | | | | | | | | | | | | 2/F-5 | | | | |
| Example 2-6 | X-4 | 10.0 | A-2 | 86.1 | B-4 | 3.0 | — | — | D-4 | 0.9 | — | — | F-1/F-2 | 80/20 | A | A | C |
| Example 2-7 | X-5 | 25.0 | A-1 | 74.1 | — | — | — | — | D-1 | 0.9 | — | — | F-1/F-2 | 80/20 | A | B | A |
| Example 2-8 | X-5 | 22.0 | A-8 | 72.4 | — | — | — | — | D-1 | 5.5 | E-1 | 0.1 | F-1/F-2 | 40/60 | A | B | A |
| Example 2-9 | X-6 | 16.0 | A-2 | 82.5 | — | — | — | — | D-5 | 1.5 | — | — | F-1/F-2 | 90/10 | A | C | B |
| Example 2-10 | X-7 | 17.0 | A-4 | 79.2 | — | — | — | — | D-2 | 3.8 | — | — | F-1/F-2 | 70/30 | A | A | A |
| Example 2-11 | X-8 | 16.0 | A-1 | 81.1 | — | — | — | — | D-5 | 2.9 | — | — | F-1/F-3 | 90/10 | A | A | A |
| Example 2-12 | X-8 | 18.0 | A-4 | 77.8 | — | — | C-1 | 1.0 | D-5 | 3.2 | — | — | F-1/F-2 | 80/20 | A | A | A |
| Example 2-13 | X-9 | 10.0 | A-1 | 84.9 | — | — | C-2 | 2.0 | D-3 | 3.1 | — | — | F-1/F-2 | 50/50 | A | A | C |
| Example 2-14 | X-10 | 22.0 | A-1 | 76.7 | — | — | — | — | D-5 | 1.3 | — | — | F-1/F-2 | 80/20 | A | A | B |
| Example 2-15 | X-10 | 20.0 | A-4 | 75.7 | B-1 | 3.0 | — | — | D-5 | 1.3 | — | — | F-1/F-4 | 50/50 | A | A | B |
| Example 2-16 | X-11 | 18.0 | A-4 | 81.1 | — | — | — | — | D-4 | 0.9 | — | — | F-1/F-2 | 70/30 | A | A | A |
| Example 2-17 | X-12 | 17.0 | A-3 | 79.9 | — | — | — | — | D-5 | 3.1 | — | — | F-1/F-2 | 70/30 | A | A | A |
| Example 2-18 | X-13 | 15.0 | A-2 | 84.1 | — | — | — | — | D-4 | 0.9 | — | — | F-1/F-2 | 80/20 | A | C | C |
| Example 2-19 | X-14 | 18.0 | A-5 | 80.1 | — | — | C-3 | 1.0 | D-1 | 0.9 | — | — | F-1/F-4 | 85/15 | A | A | A |
| Example 2-20 | X-15 | 15.0 | A-2 | 79.5 | — | — | — | — | D-3 | 5.5 | — | — | F-1/F- | 70/25/5 | A | A | C |
| | | | | | | | | | | | | | 2/F-3 | | | | |
| Example 2-21 | X-16 | 5.0 | A-8 | 76.5 | B-1 | 15.0 | C-4 | 0.5 | D-1 | 2.9 | E-1 | 0.1 | F-1/F-2 | 80/20 | A | A | C |
| Comparative | — | — | A-2 | 72.2 | B-1 | 20.0 | C-4 | 5.0 | D-1 | 2.8 | — | — | F-1/F-2 | 80/20 | A | C | D |
| Example 2-1 | | | | | | | | | | | | | | | | | |
| Comparative | — | — | A-4 | 74.9 | B-2 | 20.0 | C-4 | 4.0 | D-2 | 1.1 | — | — | F-1/F-2 | 70/30 | B | C | D |
| Example 2-2 | | | | | | | | | | | | | | | | | |
| Comparative | — | — | A-3 | 73.8 | B-1 | 5.0 | — | — | D-2 | 1.2 | — | — | F-1/F-2 | 80/20 | B | D | D |
| Example 2-3 | | | | | B-3 | 20.0 | | | | | | | | | | | |

TABLE 3

| | | | | | | | Acid | | | | | | | | | | |
|---|---|---|---|---|---|---|---|---|---|---|---|---|---|---|---|---|---|
| | | | | | | | diffusion | | | | | | | | | | |
| | Compound | | | | Photoacid | | control | | Hydrophobic | | Surfactant | | | | Performance | | |
| | (I) | | Resin A | | generator B | | agent C | | resin D | | E | | Solvent F | | | Preser- | |
| | | % by | | % by | | % by | | % by | | % by | | % by | | Mass | | vation | Pattern |
| | Type | mass | Type | mass | Type | mass | Type | mass | Type | mass | Type | mass | Type | ratio | Scum | stability | shape |
| Example 3-1 | X-1 | 20.0 | A-7 | 75.8 | — | — | — | — | D-1 | 4.2 | — | — | F-1/F-2 | 80/20 | A | B | A |
| Example 3-2 | X-2 | 16.0 | A-6 | 84.0 | — | — | — | — | — | — | — | — | F-1/F-2 | 70/30 | A | B | A |
| Example 3-3 | X-3 | 14.5 | A-3 | 82.7 | — | — | — | — | D-5 | 2.8 | — | — | F-1/F- | 70/25/5 | A | C | B |
| | | | | | | | | | | | | | 2/F-5 | | | | |
| Example 3-4 | X-4 | 10.0 | A-7 | 85.0 | B-5 | 5.0 | — | — | — | — | — | — | F-1/F-2 | 80/20 | A | A | C |
| Example 3-5 | X-5 | 22.0 | A-6 | 75.9 | — | — | C-1 | 2.0 | — | — | E-1 | 0.1 | F-1/F-2 | 40/60 | A | B | A |
| Example 3-6 | X-6 | 16.0 | A-2 | 84.0 | — | — | — | — | — | — | — | — | F-1/F-2 | 90/10 | A | C | B |
| Example 3-7 | X-7 | 17.0 | A-4 | 83.0 | — | — | — | — | — | — | — | — | F-1/F-2 | 70/30 | A | A | A |
| Example 3-8 | X-8 | 16.0 | A-6 | 84.0 | — | — | — | — | — | — | — | — | F-1/F-3 | 90/10 | A | A | A |
| Example 3-9 | X-9 | 10.0 | A-6 | 88.0 | — | — | C-2 | 2.0 | — | — | — | — | F-1/F-2 | 50/50 | A | A | C |
| Example 3-10 | X-10 | 22.0 | A-7 | 78.0 | — | — | — | — | — | — | — | — | F-1/F-2 | 80/20 | A | A | B |
| Example 3-11 | X-11 | 18.0 | A-7 | 82.0 | — | — | — | — | — | — | — | — | F-1/F-2 | 70/30 | A | A | A |
| Example 3-12 | X-12 | 17.0 | A-7 | 83.0 | — | — | — | — | — | — | — | — | F-1/F-2 | 70/30 | A | A | A |
| Example 3-13 | X-13 | 15.0 | A-6 | 84.0 | — | — | — | — | D-4 | 0.9 | E-1 | 0.1 | F-1/F-2 | 80/20 | A | C | C |
| Example 3-14 | X-15 | 15.0 | A-1 | 79.5 | — | — | — | — | D-3 | 5.5 | — | — | F-1/F- | 70/25/5 | A | A | C |
| | | | | | | | | | | | | | 2/F-3 | | | | |
| Comparative | — | — | A-6 | 74.9 | B-1 | 20.0 | C-4 | 5.0 | — | — | E-1 | 0.1 | F-1/F-2 | 80/20 | A | C | D |
| Example 3-1 | | | | | | | | | | | | | | | | | |
| Comparative | — | — | A-6 | 76.0 | B-2 | 20.0 | C-4 | 4.0 | — | — | — | — | F-1/F-2 | 70/30 | B | C | D |
| Example 3-2 | | | | | | | | | | | | | | | | | |
| Comparative | — | — | A-6 | 74.9 | B-1 | 5.0 | — | — | — | — | E-1 | 0.1 | F-1/F-2 | 80/20 | B | D | D |
| Example 3-3 | | | | | B-3 | 20.0 | | | | | | | | | | | |
| Example 4-1 | X-1 | 20.0 | A-7 | 75.8 | — | — | — | — | D-1 | 4.2 | — | — | F-1/F-2 | 80/20 | A | B | A |
| Example 4-2 | X-2 | 16.0 | A-6 | 84.0 | — | — | — | — | — | — | — | — | F-1/F-2 | 70/30 | A | B | A |
| Example 4-3 | X-3 | 14.5 | A-3 | 82.7 | — | — | — | — | D-5 | 2.8 | — | — | F-1/F- | 70/25/5 | A | C | B |
| | | | | | | | | | | | | | 2/F-5 | | | | |
| Example 4-4 | X-4 | 10.0 | A-7 | 85.0 | B-5 | 5.0 | — | — | — | — | — | — | F-1/F-2 | 80/20 | A | A | C |
| Example 4-5 | X-5 | 22.0 | A-6 | 75.9 | — | — | C-1 | 2.0 | — | — | E-1 | 0.1 | F-1/F-2 | 40/60 | A | B | A |
| Example 4-6 | X-6 | 16.0 | A-2 | 84.0 | — | — | — | — | — | — | — | — | F-1/F-2 | 90/10 | A | C | B |
| Example 4-7 | X-7 | 17.0 | A-4 | 83.0 | — | — | — | — | — | — | — | — | F-1/F-2 | 70/30 | A | A | A |
| Example 4-8 | X-8 | 16.0 | A-6 | 84.0 | — | — | — | — | — | — | — | — | F-1/F-3 | 90/10 | A | A | A |
| Example 4-9 | X-9 | 10.0 | A-6 | 88.0 | — | — | C-2 | 2.0 | — | — | — | — | F-1/F-2 | 50/50 | A | A | C |
| Example 4-10 | X-10 | 22.0 | A-7 | 78.0 | — | — | — | — | — | — | — | — | F-1/F-2 | 80/20 | A | A | B |
| Example 4-11 | X-11 | 18.0 | A-7 | 82.0 | — | — | — | — | — | — | — | — | F-1/F-2 | 70/30 | A | A | A |
| Example 4-12 | X-12 | 17.0 | A-7 | 83.0 | — | — | — | — | — | — | — | — | F-1/F-2 | 70/30 | A | A | A |
| Example 4-13 | X-13 | 15.0 | A-6 | 84.0 | — | — | — | — | D-4 | 0.9 | E-1 | 0.1 | F-1/F-2 | 80/20 | A | C | C |
| Example 4-14 | X-15 | 15.0 | A-1 | 79.5 | — | — | — | — | D-3 | 5.5 | — | — | F-1/F- | 70/25/5 | A | A | C |
| | | | | | | | | | | | | | 2/F-3 | | | | |
| Comparative | — | — | A-6 | 74.9 | B-1 | 20.0 | C-4 | 5.0 | — | — | E-1 | 0.1 | F-1/F-2 | 80/20 | A | C | D |
| Example 4-1 | | | | | | | | | | | | | | | | | |
| Comparative | — | — | A-6 | 76.0 | B-2 | 20.0 | C-4 | 4.0 | — | — | — | — | F-1/F-2 | 70/30 | B | C | D |
| Example 4-2 | | | | | | | | | | | | | | | | | |
| Comparative | — | — | A-6 | 74.9 | B-1 | 5.0 | — | — | — | — | E-1 | 0.1 | F-1/F-2 | 80/20 | B | D | D |
| Example 4-3 | | | | | B-3 | 20.0 | | | | | | | | | | | |

As can be seen in Tables 2 to 3 above, the resist composition of the invention has high preservation stability. Moreover, when a fine pattern is formed using the resist composition by alkali development or organic solvent development, a good pattern shape is obtained, and the occurrence of scum can be significantly reduced. However, with the resist composition in the Comparative Examples, these performance evaluation results were insufficient.

The present invention can provide an actinic ray-sensitive or radiation-sensitive resin composition having high preservation stability, allowing a good pattern shape to be obtained when a fine pattern (particularly having a line width or space width of 50 nm or less) is formed, and capable of significantly reducing the occurrence of scum. The present invention can also provide an actinic ray-sensitive or radiation-sensitive film using the actinic ray-sensitive or radiation-sensitive resin composition, a pattern forming method, a method for manufacturing an electronic device, and a compound.

While the invention has been described in detail with reference to the specific embodiments, it will be apparent to those skilled in the art that various changes and modifications can be made without departing from the spirit and scope of the invention.

What is claimed is:

1. An actinic ray-sensitive or radiation-sensitive resin composition comprising a compound (I) that generates an acid upon irradiation with actinic rays or radiation and has an ionic structure and a zwitterionic structure.

2. The actinic ray-sensitive or radiation-sensitive resin composition according to claim 1, wherein the compound (I) is a compound including at least two anionic groups and at least one cationic group that are linked via covalent bonding.

3. The actinic ray-sensitive or radiation-sensitive resin composition according to claim 1, wherein the compound (I) is a compound including two anionic groups and one cationic group that are linked via covalent bonding.

4. The actinic ray-sensitive or radiation-sensitive resin composition according to claim 1, wherein the ionic structure and the zwitterionic structure are each independently a structure including an acid anionic group, and
wherein the compound (I) generates a plurality of acid groups having different pKa values upon irradiation with the actinic rays or radiation.

5. The actinic ray-sensitive or radiation-sensitive resin composition according to claim 1, wherein the compound (I) is a compound represented by any of the following general formulas (I)-1 to (I)-3: 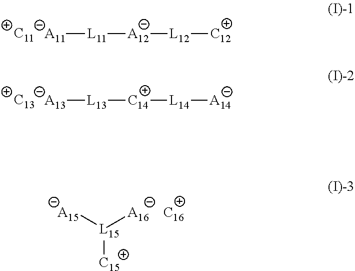
wherein, in the general formulas (I)-1 to (I)-3,
A₁₁⁻ to A₁₆⁻ each independently represent an acid anionic group;
C₁₁⁺ to C₁₆⁺ each independently represent a cationic group;
L₁₁ to L₁₄ each independently represent a divalent organic group; and
L₁₅ represents a trivalent organic group.

6. The actinic ray-sensitive or radiation-sensitive resin composition according to claim 5, wherein, in a compound PI-1 formed by replacing a counter cation of the acid anionic group represented by A₁₁⁻ in the compound represented by general formula (I)-1 with H⁺ and adding H⁺ to the acid anionic group represented by A₁₂⁻, a pKa of a group represented by A₁₁H is smaller than a pKa of a group represented by A₁₂H,
wherein, in a compound PI-2 formed by replacing a counter cation of the acid anionic group represented by A₁₃⁻ in the compound represented by general formula (I)-2 with H⁺ and adding H⁺ to the acid anionic group represented by A₁₄⁻, a pKa of a group represented by A₁₃H is smaller than a pKa of a group represented by A₁₄H, and
wherein, in a compound PI-3 formed by adding H⁺ to the acid anionic group represented by A₁₅⁻ in the compound represented by general formula (I)-3 and replacing a counter cation of the acid anionic group represented by A₁₆⁻ with H⁺, a pKa of a group represented by A₁₅H is smaller than a pKa of a group represented by A₁₆H.

7. The actinic ray-sensitive or radiation-sensitive resin composition according to claim 5, wherein A₁₁⁻ and A₁₃⁻ to A₁₆ in the formulas (I)-1 to (I)-3 each independently represent an acid anionic group represented by the following formula (A-1) or (A-2): 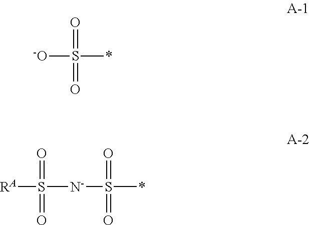
wherein, in the formulas (A-1) to (A-2),
R^{A} represents an organic group; and
* represents a bonding position.

8. The actinic ray-sensitive or radiation-sensitive resin composition according to claim 5, wherein A₁₂⁻ in the formula (I)-1 represents an acid anionic group represented by any of the following formulas (B-1) to (B-3): 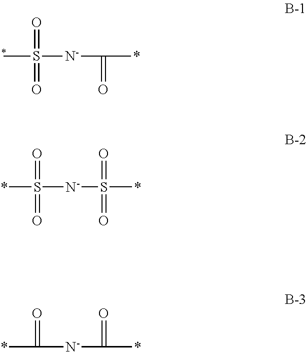
wherein, in the formulas (B-1) to (B-3),
* represents a bonding position.

9. An actinic ray-sensitive or radiation-sensitive film formed using the actinic ray-sensitive or radiation-sensitive resin composition according to claim 1.

10. A pattern forming method comprising:
forming an actinic ray-sensitive or radiation-sensitive film on a substrate using the actinic ray-sensitive or radiation-sensitive resin composition according to claim 1;
exposing the actinic ray-sensitive or radiation-sensitive film to light; and
developing the exposed actinic ray-sensitive or radiation-sensitive film with a developer.

11. A method for manufacturing an electronic device, the method comprising the pattern forming method according to claim 10.

12. A compound represented by any of the following general formulas (IA)-1 to (IA)-3: 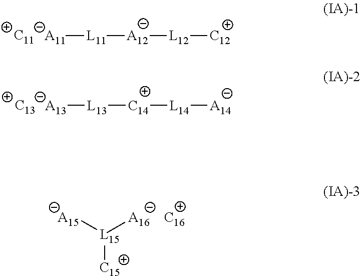
wherein, in the general formulas (IA)-1 to (IA)-3,
A₁₁⁻ and A₁₃⁻ to A₁₆⁻ each independently represent an acid anionic group represented by the formula (A-1) or (A-2) below;
A₁₂ represents an acid anionic group represented by any of the following formulas (B-1) to (B-3);
C₁₁⁺ to C₁₆⁺ each independently represent a cationic group;
L₁₁ to L₁₄ each independently represent a divalent organic group; and
L₁₅ represents a trivalent organic group; 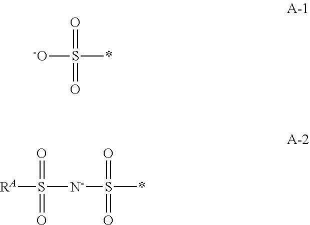
wherein, in the formulas (A-1) to (A-2),
R^{A} represents an organic group; and
* represents a bonding position; and 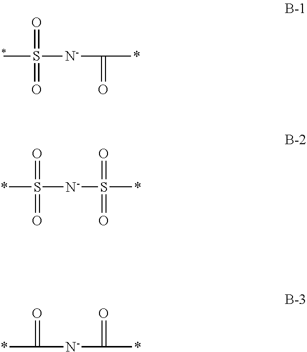
wherein, in the formulas (B-1) to (B-3),
* represents a bonding position.
